(19) United States
(12) Patent Application Publication
(10) Pub. No.: US 2008/0097557 A1
(43) Pub. Date: Apr. 24, 2008

(54) Method and apparatus for carrying out the controlled heating of tissue in the region of dermis

(75) Inventors: Philip E. Eggers, Dublin, OH (US); Andrew R. Eggers, Ostrander, OH (US); Eric A. Eggers, Portland, OR (US)

Correspondence Address: MUELLER AND SMITH, LPA;MUELLER-SMITH BUILDING, 7700 RIVERS EDGE DRIVE, COLUMBUS, OH, 43235, US

(73) Assignee: Apsara Medical Corporation, Columbus, OH (US)

(21) Appl. No.: 11/583,621

(22) Filed: Oct. 19, 2006

(51) Int. Cl. A61F 7/00; A61F 7/12

(52) U.S. Cl. 607/99; 607/113

(57) ABSTRACT

Implant apparatus and method for effecting a controlled heating of tissue within the region of dermis of skin. The heater implants are configured with a thermally insulative generally flat support functioning as a thermal barrier. One surface of this thermal barrier carries one or more electrodes within a radiofrequency excitable circuit as well as an associated temperature sensing circuit. The implants are located within heating channels at the interface between skin dermis and the next adjacent subcutaneous tissue layer such that the electrodes are contactable with the lower region of dermis. During therapy a conformal heat sink is positioned against the skin above the implants and a slight tamponade is applied through the heat sink to assure uniform dermis contact with electrode surfaces. An adjuvant may be employed to infiltrate dermis to significantly lower the thermal threshold transition temperature for dermis or dermis component shrinkage.

### CROSS-REFERENCE TO RELATED APPLICATIONS

### Statement Regarding Federally Sponsored Research

Not applicable.

### BACKGROUND

The skin or integument is a major organ of the body present as a specialized boundary lamina, covering essentially the entire external surface of the body, except for the mucosal surfaces. It forms about 8% of the body mass with a thickness ranging from about 1.5 to about 4 mm. Structurally, the skin organ is complex and highly specialized as is evidenced by its ability to provide a barrier against microbial invasion and dehydration, regulate thermal exchange, act as a complex sensory surface, and provide for wound healing wherein the epidermis responds by regeneration and the underlying dermis responds by repair (inflammation, proliferation, and remodeling), among a variety of other essential functions.

Medical specialties have evolved with respect to the skin, classically in connection with restorative and aesthetic (plastic) surgery. Such latter endeavors typically involve human aging. The major features of the skin are essentially formed before birth and within the initial two to three decades of life are observed to not only expand in surface area but also in thickness. From about the third decade of life onward there is a gradual change in appearance and mechanical properties of the skin reflective of anatomical and biological changes related to natural aging processes of the body. Such changes include a thinning of the adipose tissue underlying the dermis, a decrease in the collagen content of the dermis, changes in the molecular collagen composition of the dermis, increases in the number of wrinkles, along with additional changes in skin composition. The dermis itself decreases in bulk, and wrinkling of senescent skin is almost entirely related to changes in the dermis. Importantly, age related changes in the number, diameter, and arrangement of collagen fibers are correlated with a decrease in the tensile strength of aging skin in the human body, and the extensibility and elasticity of skin decrease with age. Evidence indicates that intrinsically aged skin shows morphological changes that are similar in a number of features to skin aged by environmental factors, including photoaging.

See generally:
1. Gray's Anatomy, 39^{th} Edition, Churchill Livingstone, New York (2005)
2. Rook's Textbook of Dermatology, 7^{th} Edition, Blackwell Science, Malden, Mass. (2004)

A substantial population of individuals seeking to ameliorate this aging process has evolved over the decades. For instance, beginning in the late 1980s researchers who had focused primarily on treating or curing disease began studying healthy skin and ways to improve it and as a consequence, a substantial industry has evolved. By reducing and inhibiting wrinkles and minimizing the effects of ptosis (skin laxity and sagging skin) caused by the natural aging of collagen fibrils within the dermis, facial improvements have been realized with the evolution of a broad variety of corrective approaches.

Considering its structure from a microscopic standpoint, the skin is composed of two primary layers, an outer epidermis which is a keratinized stratified squamous epithelium, and the supporting dermis which is highly vascularized and provides supporting functions. In the epidermis tissue there is a continuous and progressive replacement of cells, with a mitotic layer at the base replacing cells shed at the surface. Beneath the epidermis is the dermis, a moderately dense connective tissue. The epidermis and dermis are connected by a basement membrane or basal lamina with greater thickness formed as a collagen fiber which is considered a Type I collagen having an attribute of shrinking under certain chemical or heat influences. Lastly, the dermis resides generally over a layer of contour defining subcutaneous fat. Early and some current approaches to the rejuvenation have looked to treatments directed principally to the epidermis, an approach generally referred to ablative resurfacing of the skin. Ablative resurfacing of the skin has been carried out with a variety of techniques. One approach, referred to as “dermabrasion” in effect mechanically grinds off components of the epidermis.

Mechanical dermabrasion activities reach far back in history. It is reported that about 1500 B.C. Egyptian physicians used sandpaper to smooth scars. In 1905 a motorized dermabrasion was introduced. In 1953 powered dental equipment was modified to carry out dermabrasion practices. See generally:
3. Lawrence, et al., “History of Dermabrasion” Dermatol Surg 2000; 26:95-101

A corresponding chemical approach is referred to by dermatologists as “chemical peel”. See generally:
4. Moy, et al., “Comparison of the Effect of Various Chemical Peeling Agents in a Mini-Pig Model” Dermatol Surg. 22:429-432 (1996).

Another approach, referred to as “laser ablative resurfacing of skin” initially employed a pulsed CO₂ laser to repair photo-damaged tissue which removed the epidermis and caused residual thermal damage within the dermis. It is reported that patients typically experienced significant side effects following this ablative skin resurfacing treatment. Avoiding side effects, non-ablative dermal remodeling was developed wherein laser treatment was combined with timed superficial skin cooling to repair tissue defects related to photo-aging. Epidermal removal or damage thus was avoided, however, the techniques have been described as having limited efficacy. More recently, fractional photothermolysis has been introduced wherein a laser is employed to fire short, low energy bursts in a matrix pattern of non-continuous points to form a rastor-like pattern. This pattern is a formation of isolated non-continuous micro-thermal wounds creating necrotic zones surrounded by zones of viable tissue. See generally:
5. Manstein, et al., “Fractional Photothermolysis: A New Concept for Cutaneous Remodeling Using Microscopic Patterns of Thermal Injury”; Lasers in Surgery and Medicine 34:426-438 (2004)

These ablative techniques (some investigators consider fractional photothermolysis as a separate approach) are associated with drawbacks. For instance, the resultant insult to the skin may require 4-6 months or more of healing to evolve newer looking skin. That newer looking skin will not necessarily exhibit the same shade or coloration as its original counterpart. In general, there is no modification of the dermis in terms of a treatment for ptosis or skin laxity through collagen shrinkage.

To treat patients for skin laxity, some investigators have looked to procedures other than plastic surgery. Techniques for induced collagen shrinkage at the dermis have been developed. Such shrinkage qualities of collagen have been known and used for hundreds of years, the most classic example being the shrinking of heads by South American headhunters. Commencing in the early 1900s shrinking of collagen has been used as a quantitative measure of tanning with respect to leather and in the evaluation of glues See:
6. Rasmussen, et al., “Isotonic and Isometric Thermal Contraction of Human Dermis I. Technic and Controlled Study”, J. Invest. Derm. 43:333-9 (1964).

Dermis has been heated through the epidermis utilizing laser technology as well as intense pulsed light exhibiting various light spectra or single wavelength. The procedure involves spraying a burst of coolant upon the skin such as refrigerated air, whereupon a burst of photons penetrates the epidermis and delivers energy into the dermis.

Treatment for skin laxity by causing a shrinkage of collagen within the dermis generally involves a heating of the dermis to a temperature of about 60° C. to 70° C. over a designed treatment interval. Heat induced shrinkage has been observed in a course of laser dermabrasion procedures. However, the resultant energy deposition within the epidermis has caused the surface of the skin to be ablated (i.e., burned off the surface of the underlying dermis) exposing the patient to painful recovery and extended healing periods which can be as long as 6-12 months. See the following publication:
7. Fitzpatrick, et al., “Collagen Tightening Induced by Carbon Dioxide Laser Versus Erbium: YAG Laser” Lasers in Surgery and Medicine 27: 395-403 (2000).

Dermal heating in consequence of the controlled application of energy in the form of light or radiofrequency electrical current through the epidermis and into the dermis has been introduced. To avoid injury to the epidermis, cooling methods have been employed to simultaneously cool the epidermis while transmitting energy through it. In general, these approaches have resulted in uncontrolled, non-uniform and often inadequate heating of the dermis layer resulting in either under-heating (insufficient collagen shrinkage) or over heating (thermal injury) to the subcutaneous fat layer and/or weakening of collagen fibrils due to over-shrinkage. See the following publication:
8. Fitzpatrick, et al., “Multicenter Study of Noninvasive Radiofrequency for Periorbital Tissue Tightening”, Lasers in Surgery in Medicine 33:232-242 (2003).

The RF approach described in publication 8 above is further described in U.S. Pat. Nos. 6,241,753; 6,311,090; 6,381,498; and 6,405,090. Such procedure involves the use of an electrode capacitively coupled to the skin surface which causes radiofrequency current to flow through the skin to a much larger return electrode located remotely upon the skin surface of the patient. Note that the electrodes are positioned against skin surface and not beneath it. The radiofrequency current density caused to flow through the skin is selected to be sufficiently high to cause resistance heating within the tissue and reach temperatures sufficiently high to cause collagen shrinkage and thermal injury, the latter result stimulating beneficial growth of new collagen, a reaction generally referred to as “neocollagenasis”.

To minimize thermal energy to the underlying subcutaneous fat layer these heating methods also attempt to apply energy periods with pulse durations on the order of several nanoseconds to several thousand microseconds for laser based methods and several seconds for radiofrequency electrical current based methods. This highly transient approach to heating the collagen within the dermis also leads to a wide range of temperature variations due to natural patient-to-patient differences in the optical and electrical properties of their skin including localized variations in electrical properties of skin layers. It may be observed that the electrical properties of the dermis are not necessarily homogenous and may vary somewhat within the treatment zone, for example, because of regions of concentrated vascularity. This may jeopardize the integrity of the underlying fat layer and damage it resulting in a loss of desired facial contour. Such unfortunate result at present appears to be uncorrectable. Accordingly, uniform heating of the dermal layer is called for in the presence of an assurance that the underlying fat layer is not affected while minimal injury to the epidermis is achieved. A discussion of the outcome and complications of the noted non-ablative mono-polar radiofrequency treatment is provided in the following publication:
9. Abraham, et al., “Current Concepts in Nonablative Radiofrequency Rejuvenation of the Lower Face and Neck” Facial Plastic Surgery, Vol. 21 No. 1 (2005).

In the late 1990s, Sulamanidzei developed a mechanical technique for correcting skin laxity. With this approach one or more barbed non-resorbable sutures are threaded under the skin with an elongate needle. The result is retention of the skin in a contracted state and, over an interval of time, the adjacent tissue will ingrow around the suture to stabilize the facial correction. See the following publications:
10. Sulamanidze, et al., “Removal of Facial Soft Tissue Ptosis With Special Threads”, Dermatol Surg. 28:367-371 (2002).
11. Lycka, et al., “The Emerging Technique of the Antiptosis Subdermal Suspension Thread”, Dermatol Surg. 30:41-44 (2004).

Eggers, et al., in application for U.S. patent Ser. No. 11/298,420 entitled “Aesthetic Thermal Sculpting of Skin”, filed Dec. 9, 2005 describes the technique for directly applying heat energy to dermis with one or more thermal implants providing controlled shrinkage thereof. Importantly, while this heating procedure is underway, the subcutaneous fat layer is protected by a polymeric thermal barrier. In one arrangement this barrier implant is thin and elongate and supports a flexible resistive heating circuit, the metal heating components of which are in direct contact with dermis. Temperature output of this resistive heating circuit is intermittently monitored and controlled by measurement of a monitor value of resistance. For instance, resistive heating is carried out for about a one hundred millisecond interval interspersed with one millisecond resistance measurement intervals. Treatment intervals experienced with this system and technique will appear to obtain significant collagen shrinkage within about ten minutes to about fifteen minutes. During the procedure, the epidermis is cooled by blown air.

Some of the procedures described above may be carried out using local anesthesia. Local anesthetic agents are weakly basic tertiary amines, which are manufactured as chloride salts. The molecules are amphipathic and have the function of the agents and their pharmacokinetic behavior can be explained by the structure of the molecule. Each local anesthetic has a lipophilic side; a hydrophilic-ionic side; an intermediate chain, and, within the connecting chain, a bond. That bond determines the chemical classification of the agents into esters and amides. It also determines the pathway for metabolism. While there are a variety of techniques for administering local anesthesia, in general, it may be administered for infiltration, activity or as a nerve block. In each approach, the active anesthetic drug is administered for the purpose of intentionally interrupting neural function and thereby providing pain relief.

A variety of local anesthetics have been developed, the first agent for this purpose being cocaine which was introduced at the end of the nineteenth century. Lidocaine is the first amide local anesthetic and the local anesthetic agent with the most versatility and thus popularity. It has intermediate potency, toxicity, onset, and duration, and it can be used for virtually any local anesthetic application. Because of its widespread use, more knowledge is available about metabolic pathways than any other agent. Similarly, toxicity is well known.

Vasoconstrictors have been employed with the local anesthetics. In this regard, epinephrine has been added to local anesthetic solutions for a variety of reasons throughout most of the twentieth century to alter the outcome of conduction blockade. Its use in conjunction with infiltration anesthesia consistently results in lower plasma levels of the agent. See generally:
12. “Clinical Pharmacology of Local Anesthetics” by Tetzlaff, J. E., Butterworth-Heinemann, Woburn, Mass. (2000).

To minimize the possibility of irreversible nerve injury in the course of using local anesthetics, the drugs necessarily are diluted. By way of example, the commonly used anesthetic drug is injected using concentrations typically in the range of 0.4% to 2.0% (weight percent). The diluent contains 0.9% sodium chloride. Such isotonic saline is used as the diluent due to the fact that its osmolarity at normal body temperature is 286 milliOsmols/liter which is close to that of cellular fluids and plasma which have a osmolarity of 310 milliOsmols/liter. As a result, the osmotic pressure developed across the semipermeable cell membranes is minimal when isotonic saline is injected. Consequently, there is no injury to the tissue's cells surrounded by this diluent since there is no significant gradient which can cause fluids to either enter or leave the cells surrounded by the diluent. It is generally accepted that diluents having an osmolarity in the range of 240 to 340 milliOsmols/liter are isotonic solutions and therefore can be safely injected.

Dermis also is the situs of congenital birthmarks generally deemed to be capillary malformations historically referred to as “Port-Wine Stains” (PWS). Ranging in coloration from pink to purple, these non-proliferative lesions are characterized histologically by ecstatic vessels of capillary or venular type within the papillary and reticular dermis and are considered as a type of vascular malformation. The macular lesions are relatively rare, occurring in about 0.3% of newborns and generally appear on the skin of the head and neck within the distribution of the trigeminal (fifth cranial) nerve. They persist throughout life and may become raised, nodular, or darken with age. Their depth has been measured utilizing pulsed photothermal radiometry (PPTR) and ranges from about 200 μm to greater than 1000 μm.

See the following publication:
13. Bincheng, et al., Accurate Measurement of Blood Vessel Depth in Port Wine Stain Human Skin in vivo Using “Photothermal Radiometry”, J. Biomed. Opt. (5), 961-966 (September/October 2004).

Fading or lightening the PWS lesions has been carried out with lasers with somewhat mixed results. For instance, they have been treated with pulsed dye lasers (PDL) at 585 mm wavelength with a 0.45 ms pulse length and 5 mm diameter spot size. Cryogenic bursts have been used with the pulsing for epidermal protection. Generally, the extent of lightening achieved is evaluated six to eight weeks following laser treatment. Such evaluation assigns the color of adjacent normal skin as 100% lightening and a post clearance, evaluation of lesions will consider more than 75% lightening as good.

See the following publication:
14. Fiskerstrand, et al., “Laser Treatment of Port Wine Stains: Thereaupetic Outcome in Relation to Morphological Parameters” Brit. J. of Derm., 134, 1039-1043, (1996).

Lesions have been classified, for instance, utilizing video microscopy, three patterns of vascular ectasia being established; type 1, ectasia of the vertical loops of the papillary plexus; type 2, ectasia of the deeper, horizontal vessels in the papillary plexus; and type 3, mixed pattern with varying degrees of vertical and horizontal vascular ectasia. In general, due to the limited depth of laser therapy, only type 1 lesions are apt to respond to such therapy.

Port wine stains also are classified in accordance with their degree of vascular ectasia, four grades thereof being recognized, Grades I to IV.

Grade 1 lesions are the earliest lesions and thus have the smallest vessels (50-80 um in diameter). Using ×6 magnification and transillumination, individual vessels can only just be discerned and appear like grains of sand. Clinically, these lesions are light or dark pink macules. Grade II lesions are more advanced (vessel diameter=80-120 um). Individual vessels are clearly visible to the naked eye, especially in less dense areas. They are thus clearly distinguishable macules. Grade III lesions are more ecstatic (120-150 um). By this stage, the space between the vessels has been replaced by the dilated vessels. Individual vessels may still be visible on the edges of the lesion or in a less dense lesion, but by and large individual vessels are no longer visible. The lesion is usually thick, purple, and palpable. Eventually dilated vessels will coalesce to form nodules, otherwise known as cobblestones. Grade IV represents the largest vessels. The main purpose of these classifications has been to assign a grade for ease in communication and determination of the appropriate laser treatment settings.

See the following publication:
15. Mihm, Jr., et al, “Science, Math and Medicine—Working Together to Understand the Diagnosis, Classification and Treatment of Port-Wine Stains”, a paper presented in Mt. Tremblant, Quebec, Canada, 2004, Controversies and Conversations in Cutaneous Laser Surgery—An Advanced Symposium.

### BRIEF SUMMARY

The present disclosure is addressed to embodiments of apparatus and methods for effecting a controlled heating of tissue within the region of the dermis of skin using heater implants that are configured with a thermally insulative generally flat support functioning as a thermal barrier. One surface of this thermal barrier carries one or more electrodes within a radiofrequency excitable circuit as well as an associated temperature sensing circuit arranged to monitor the temperature levels of the electrodes. When in use, the implants are located within heating channels at the interface between skin dermis and the next adjacent subcutaneous tissue layer sometimes referred to as a contour defining fat layer. With such positioning, the electrodes are contactable with the lower region of dermis while the flat polymeric support functions as a thermal barrier importantly enhancing the protection of the next adjacent subcutaneous tissue layer from thermal damage. Research is described showing that, by applying a slight pressure or tamponade to the skin surface over the implants, substantially improved electrical performance is realized. For instance, where the implants are used for skin remodeling calling for temperature generation at or above the thermal threshold for dermis or dermis component based skin shrinkage, the therapy interval may be designed to be of very practical length and substantially uniform regional heating is achieved. Control of skin surface temperature during therapy is carried out with heat sinks preferably having a conformal contact surface performing in concert with an interposed thermal energy transfer medium which typically is a liquid such as water. One heat sink configuration includes a flexible, bag-like transparent polymeric container which carries a heat sinking fluid such as water. Heat transfer performance of the devices is improved by agitating the liquid within the container, and a variety of techniques for such liquid action are described. Other energy transfer mediums include water-based solutions such as isotonic saline, antimicrobial solutions as well as alcohols, isopropyl alcohol, or oils, e.g., mineral oil. The heat sinks may be employed to assert the noted tamponade and, when transparent, permit visual monitoring of the extent of remodeling skin shrinkage. The ideal therapy intervals permit the practitioner to observe the shrinkage as it occurs.

In general, skin remodeling is carried out with bipolar excitation between the electrodes of two or more implants with setpoint temperatures at or above the thermal threshold transition temperature for carrying out the shrinkage of dermis or components of dermis. Advantageously, that thermal threshold transition temperature may be reduced, for example, to the extent of about 10° C. to about 12° C. by pre-administering an adjuvant to infuse into the dermis. Such adjuvant may be one or more of a salt, an enzyme, a detergent, a lipophile, a denaturing solvent, an organic denaturant, an acidic solution, or a basic solution.

The implants and associated method also may be employed for the treatment of a capillary malformation sometimes referred to as “port wine stain” (PWS). For this application, implant based heating is carried out to effect an irreversible vascular coagulation at a setpoint temperature which is atraumatic to the dermis and epidermis.

In addition to the bipolar excitation of paired electrodes of the implants, excitation may be implemented under a quasi-bipolar approach. With this approach, the electrodes of the implants perform in concert with a current diffusing return electrode which is positioned in electrical return relationship against skin over the implants. With the arrangement, current flow is away from the next adjacent subcutaneous tissue or fat layer and the positioning of the implants becomes more flexible. Such return electrode may be implemented as a thin, flexible electrically conductive contact surface of a polymeric conformal heat sink.

In general, bipolar excitation of paired electrodes is undertaken with an initial power ramping over a ramp interval to a setpoint temperature, whereupon the radiofrequency-based power level is reduced and what is referred to as a “soaking interval” ensues for the completion of the therapy interval.

Alternately, bipolar excitation of paired electrodes may be undertaken at a fixed applied power level (or current level) until the electrode temperatures reach a first setpoint at which time the power (or current) is reduced to some fraction of the initial power (or current), e.g., to 50% until the final temperature setpoint is attained, which may be maintained for an additional “soaking interval”.

Other objects of the disclosure of embodiments will, in part, be obvious and will, in part, appear hereinafter.

The instant presentation, accordingly, comprises embodiments of the apparatus and method possessing the construction, combination of elements, arrangement of parts and steps which are exemplified in the following detailed disclosure.

For a fuller understanding of the nature and objects herein involved, reference should be made to the following detailed description taken in connection with the accompanying drawings.

### BRIEF DESCRIPTION OF THE DRAWINGS

FIG. 1 is a diagram of the structure of the extra cellular matrix of dermis tissue;

FIG. 2 is a family of curves relating linear shrinkage of dermis with time and temperature;

FIG. 3 is a schema representing the organization of skin;

FIG. 4 is a perspective view of an experimental implant combining a thermal barrier, platinum electrode and thermocouple;

FIG. 5 is a sectional view taken through the plane 5-5 shown in FIG. 4;

FIG. 6 is a schematic and perspective representation of ex vivo experimentation utilizing two implants as described in connection with FIGS. 4 and 5;

FIG. 7 is an end view of the schematic representation of FIG. 6;

FIG. 8 is a schematic depiction of the relationship of cell death with respect to temperature and time;

FIG. 9 is a schematic representation of the relationship of tissue resistance with RF power and time;

FIG. 10 is a schematic representation of constant applied RF power and the relationship of tissue resistance with power and time;

FIG. 11 is a representation of a dot matrix pattern on in vivo animal skin at the commencement of an experiment, the figure also showing digitally recorded locations of such dots;

FIG. 12 is a representation of the image of FIG. 11 following 40 seconds of RF implant heating of dermis;

FIG. 13 is a representation of the image of FIG. 11 showing relative positioning of image dots at time 60 seconds in the experiment under a condition in which power was turned off at approximately 55 seconds;

FIG. 14 is a top schematic view of an experimental procedure wherein current flux concentrations were determined to be present;

FIG. 15 is a sectional view taken through the plane 15-15 in FIG. 14;

FIG. 16 is a perspective schematic representation of experimentation undertaken utilizing two implants as described in connection with FIGS. 4 and 5 in conjunction with a liquid-filled conformal heat sink and a glass plate for applying pressure;

FIG. 17 is a top schematic view of the experiment of FIG. 16;

FIG. 18 is a sectional view taken through the plane 18-18 shown in FIG. 17;

FIG. 19 is a schematic and perspective view of an experimentation carried out utilizing an instrumented and heated aluminum heat sink;

FIG. 20 is a sectional view taken through the plane 20-20 shown in FIG. 19;

FIG. 21 is an enlarged partial view of an identified portion of the section of FIG. 20;

FIG. 22 are curves relating temperature with time which are computationally developed and show a pre-cooling function, a therapy function and a post therapy function;

FIG. 23 is a graph relating RF power level, setpoint and electrode temperature with time and showing a reduction in power level as electrode temperature reaches setpoint temperature;

FIG. 24 is a perspective view of a single electrode implant;

FIG. 25 is a partial perspective view of the leading end of the implant of FIG. 24;

FIG. 26 is a top view of the implant of FIG. 24;

FIG. 27 is a bottom view of the implant of FIG. 24;

FIG. 28 is a sectional view taken through the plane 28-28 shown in FIG. 26;

FIG. 29 is an enlarged partial top view of the implant of FIG. 24;

FIG. 30 is an enlarged partial top view of the trailing end of the implant of FIG. 24;

FIG. 31A is an enlarged partial view of a temperature sensing resistor segment supported upon a substrate;

FIG. 31B is an enlarged view of the substrate of FIG. 31A showing the trailing end region thereof;

FIG. 32 is a perspective view of the upward side of a cable connector guide employed with the implant of FIG. 24;

FIG. 33 is a perspective view of the cable connector guide of FIG. 32 but showing its underside;

FIG. 34 is a perspective view of an implant supporting four RF electrodes;

FIG. 35 is a top view of the implant of FIG. 34;

FIG. 36 is a bottom view of the implant shown in FIG. 34;

FIG. 37 is an enlarged broken away top view of the forward region of the implant of FIG. 34;

FIG. 38 is an enlarged top view showing the lead components located at the trailing end of the implant of FIG. 34;

FIG. 39 is an enlarged broken away view of the inward side of the substrate component of the implant of FIG. 34;

FIG. 40 is an enlarged view of the trailing end of the substrate shown in FIG. 39;

FIG. 41 is an exploded view of the connector guide shown in FIG. 34 also revealing the thermal barrier and upper lead structure;

FIG. 42 is a partial perspective view of the bottom trailing end region of the thermal barrier and associated circuit of the implant of FIG. 34;

FIG. 43 is a partial perspective view of the connector guide shown in FIG. 41 and further showing its connection with a cable connector;

FIG. 44 is a sectional view taken through the plane 44-44 shown in FIG. 43;

FIG. 45 is a top view of a blunt dissector introducer;

FIG. 46 is a side view of the introducer of FIG. 45;

FIG. 47 is a top schematic view of a transparent heat sink showing a water agitating pneumatic bladder;

FIG. 48 is a sectional view taken through the plane 48-48 shown in FIG. 47;

FIG. 49 is a schematic sectional view of the heat sink shown in FIG. 47 and showing a controller arrangement associated therewith as well as an expanded water agitating bladder;

FIG. 50 is a top schematic view of a transparent conformal heat sink utilizing temperature controlled water recirculation;

FIG. 51 is a top schematic view of a conformal transparent heat sink showing temperature controlled water recirculation in conjunction with a water driven agitator;

FIG. 52 is a top schematic view of a conformal transparent heat sink retaining water agitated with a motor driven magnetic stirring assembly;

FIG. 53 is a sectional view taken through the plane 53-53 shown in FIG. 52;

FIG. 54 is a top schematic view of a conformal transparent heat sink, the water within which is agitated by a motor driven impeller;

FIG. 55 is a sectional view taken through the plane 55-55 shown in FIG. 54;

FIG. 56 is a perspective schematic representation of skin, the upper surface of which is being marked to provide a visible dot matrix;

FIG. 57 is a perspective view showing the skin or FIG. 56 with the interior of the contact surface of a conformal transparent heat sink being marked with dots which coincide with those shown in FIG. 56;

FIG. 58 is a top schematic view of a conformal transparent heat sink showing the inward side of its contact surface carrying a grid having intersections matching a skin carried dot matrix;

FIG. 59 is a top schematic view of a transparent conformal heat sink, the contact surface of which is coated with a thermochromic material and showing a region of heat induced coloration of such material;

FIG. 60 is a schematic representation of a controller performing in conjunction with two parallel spaced-apart four channel implants;

FIG. 61 is a schematic top view showing the relative spacing of four, four channel electrodes as they are implanted;

FIG. 62A is a partial sectional view showing the implants of FIG. 61 located at the intersection between dermis and adjacent subcutaneous tissue;

FIG. 62B an energization versus time diagram is presented describing an energization of the four electrode implants of FIG. 62A;

FIG. 63 is an enlarged broken away and partial view of a substrate supported sequence of four resistor segments which are employed both for temperature sensing and heating;

FIG. 64 is an enlarged partial view of the trailing end of the substrate of FIG. 63 showing a direct lead connection with the resistor segments;

FIG. 65 is a partial sectional schematic view showing the utilization of the hybrid arrangement of FIG. 63 and FIG. 64 in connection with border located implants;

FIG. 66 is a schematic representation of the control associated with the operation of four implants as described in conjunction with FIG. 65;

FIG. 67 is a top view of a bladed implant;

FIG. 68 is a perspective view of a blunt dissection blade employed with implants as at FIG. 67;

FIG. 69 is a partial top view of a thermal barrier within which a blade component as shown in FIG. 68 has been imbedded;

FIG. 70 is a sectional view taken through the plane 70-70 shown in FIG. 69;

FIG. 71A is a sectional schematic view of skin showing bipolar energization sequence between first and third implants;

FIG. 71B is a schematic sectional view as seen in FIG. 71A but showing bipolar energization between second and fourth implants;

FIG. 71C is an energization sequence associated with FIGS. 71A and 71B;

FIG. 72A is a schematic sectional view of skin showing three implants and bipolar energization between first and third implants;

FIG. 72B is a schematic sectional representation as shown in FIG. 72A but illustrating a first and second and second and third energization sequence for the implants;

FIG. 72C is an energization diagram describing the energization illustrated in FIGS. 72A and 72B;

FIG. 73 is a perspective view of a single implant with spaced apart bipolar electrodes;

FIG. 74 is a schematic sectional view showing a current flux path developed with the implant of FIG. 73;

FIG. 75 is a block diagrammatic representation of a controller performing with the implants of the invention;

FIG. 76 is a block schematic representation of the performance of three, four channel implants;

FIG. 77 is a schematic curve set relating electrode temperature and time with respect to a controlled ramp-up of power to a setpoint temperature followed by a thermal soak interval at a reduced constant power, two setpoint temperatures being illustrated;

FIG. 78 is a schematic representation of ex vivo experiments undertaken with three experimental implants;

FIG. 79A is a schematic top view of an implant of predetermined length supporting four electrodes of about 15 mm length;

FIG. 79B is a schematic top view of another implant having the same predetermined length but supporting four electrodes of length of about 12 mm;

FIG. 79C is a schematic top view of an implant having the same length as the implants shown in FIGS. 79A and 79B but showing four electrodes having a length of about 8 mm;

FIG. 80 is an exploded perspective view showing an implant thermal barrier associated with two layers of substrate, one carrying electrodes and the other carrying temperature sensing resistor segments;

FIG. 81 is a perspective view of the implant of FIG. 80 showing its combination with a connector guide;

FIG. 82 is a sectional view taken through the plane 82-82 shown in FIG. 81;

FIG. 83 is a partial sectional view taken through the plane 83-83 in FIG. 81 with respect to the connector guide and further showing a partial sectional view of a commercially available cable connector;

FIGS. 84A-84I combine as labeled thereon to provide a flow chart of procedure according to the invention for carrying out shrinkage of collagen at dermis;

FIGS. 85A-85B combine as labeled thereon to provide a flow chart illustrating procedures for carrying out thermal treatment of capillary malformation lesions;

FIG. 86 is a schematic sectional view of a quasi-bipolar utilization of implants according to the invention;

FIG. 87 is a partial top view of the arrangement of FIG. 86 showing an orientation of two implants in phantom;

FIG. 88 is a flow chart stemming from node B in FIG. 85A and returning thereto at node C; and

FIG. 89 is a flow chart stemming from node D. in FIG. 85D and returning thereto at node E.

### DETAILED DESCRIPTION OF THE INVENTION

The discourse to follow will reveal that the system, method and implants described were evolved over a sequence of animal (pig) experiments, both ex vivo and in vivo. In this regard, certain of the experiments and their results are described to, in effect, set forth a form of invention history giving an insight into the reasoning under which the embodiments developed.

The arrangement of the physical structure of the dermis is derived in large part from the structure of the extracellular matrix surrounding the cells of the dermis. The term extra cellular matrix refers collectively to those components of a tissue such as the dermis that lie outside the plasma membranes of living cells, and it comprises an interconnected system of insoluble protein fibers, cross-linking adhesive glycoproteins and soluble complexes of carbohydrates and carbohydrates covalently linked to proteins (e.g. proteoglycans). A basement membrane lies at the boundary of the dermis and epidermis, and is structurally linked to the extracellular matrix of the dermis and underlying hypodermis. Thus the extracellular matrix of the dermis distributes mechanical forces from the epidermis and dermis to the underlying tissue.

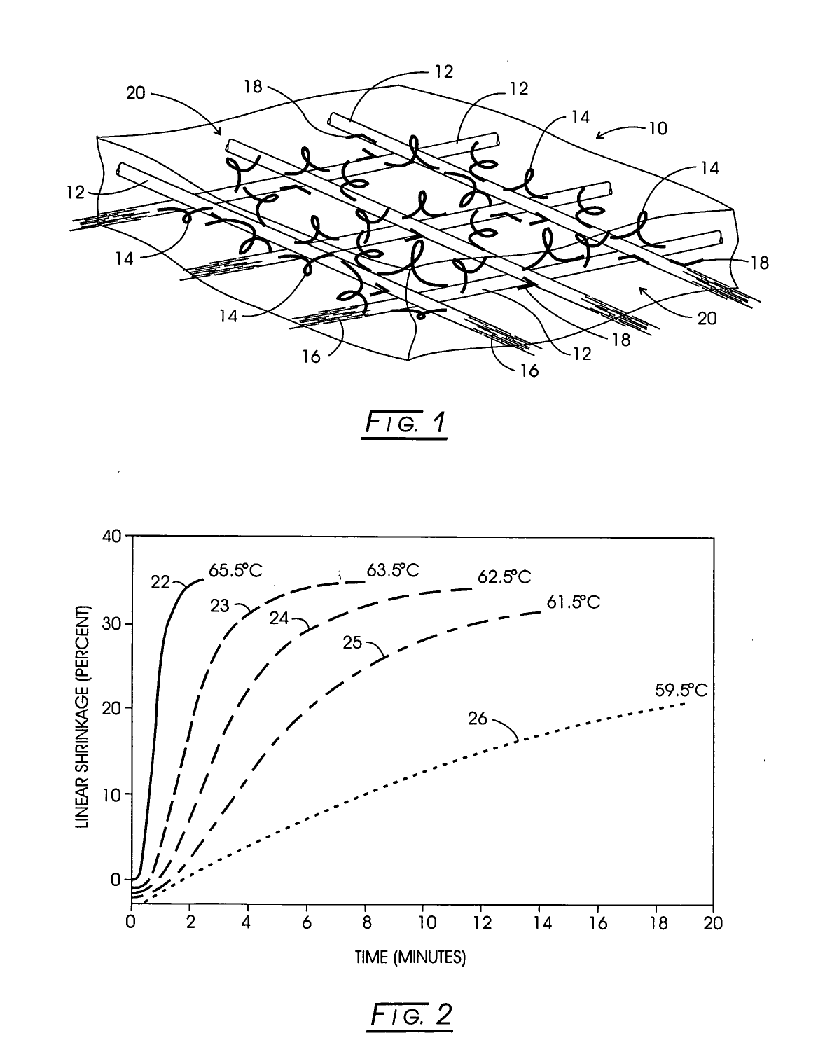

Looking to FIG. 1, a schematic representation of a region of the extracellular matrix of the dermis is represented generally at 10. The insoluble fibers include collagen fibers at 12, most commonly collagen Type I, and elastin at 14. The fundamental structural unit of collagen is a long, thin protein (300 nm×15 nm) composed of three subunits coiled around one another to form the characteristic right-handed collagen triple helix. Collagen is formed within the cell as procollagen, wherein the three subunits are covalently cross linked to one another by disulphide bonds, and upon secretion are further processed into tropocollagen. The basic tropocollagen structure consists of three polypeptide chains coiled around each other in which the individual collagen molecules are held in an extended conformation. The extended conformation of a tropocollagen molecule is maintained by molecular forces including hydrogen bonds, ionic interactions, hydrophobicity, salt links and covalent cross-links. Tropocollagen molecules are assembled in a parallel staggered orientation into collagen fibrils at 16, each containing a large number of tropocollagens, held in relative position by the above listed molecular forces and by cross-links between hydrolysine residues of overlapping tropocollagen molecules. Certain aspects of collagen stabilization are enzyme mediated, for example by Cu-dependent lysyl oxidase. Collagen fibrils are typically of about 50 nm in diameter. Type I collagen fibrils have substantial tensile strength, greater on a weight basis than that of steel, such that the collagen fibril can be stretched without breaking. Collagen fibrils are further aggregated into more massive collagen fibers, as previously shown at 12. The aggregation of collagen fibers involves a variety of molecular interactions, such that it appears that collagen fibers may vary in density based on the particular interactions present when formed. Elastin, in contrast to collagen, does not form such massive aggregated fibers, may be thought of as adopting a looping conformation (as shown at 14) and stretch more easily with nearly perfect recoil after stretching.

The extracellular matrix (ECM) as at 10 lies outside the plasma membrane, between the cells forming skin tissue. The components of the ECM including tropocollagen, are primarily synthesized inside the cells and then secreted into the ECM through the plasma membrane. The overall structure and anatomy of the skin, and in particular the dermis, are determined by the close interaction between the cells and ECM. Referring again to FIG. 1, only a few of the many and diverse components of the ECM are shown. In addition to collagen fibers 12 and elastin 14 are a large number of other components that serve to crosslink or cement these named components to themselves and to other components of the ECM. Such crosslinking components are represented generally as at 18, and may be of protein, glycoprotein and or carbohydrate composition, for example. The cross linked collagen fibers shown in FIG. 1 are embedded in a layer of highly hydrated material, including a diverse variety of modified carbohydrates, including particularly the large carbohydrate hyaluronic acid (hyaluronan) and chondroitin sulphate. Hyaluronan is a very large, hydrated, non-sulphated mucopolysaccaride that forms highly viscous fluids. Chondroitin sulphate is a glycosaminoglycan component of the ECM. Accordingly, the volume of the ECM as represented generally at 20 is filled with a flexible gel with a hydrated hyaluronan component that surrounds and supports the other structural components such as collagen and elastin. Thus the structural form of the dermis may be thought to be composed of collagen, providing tensile strength, with the collagen being held in place within a matrix of hyaluronan, which resists compression. Underlying this structure are the living cells of the dermis, which in response to stimuli (such as wounds or stress, for instance) can be induced to secrete additional components, synthesize new collagen (i.e. neocollagenesis), and otherwise alter the structural form of the ECM and the skin itself. The structure of the collagen reinforced connective tissues should not be considered entirely static, but rather that the net accumulation of collagen connective tissues is an equilibrium between synthesis and degradation of the components of the collagen reinforced connective tissues. Similarly, the other components of the ECM are modulated in response to environmental stimuli.

As noted earlier previous researchers have shown that collagen fibers can be induced to shrink in overall length by application of heat. Experimental studies have reported that collagen shrinkage is, in fact, dependent upon the thermal dose (i.e., combination of time and temperature) in a quantifiable manner. (See publication 16, infra). Looking to FIG. 2, a plot of linear collagen shrinkage versus time for various constant temperatures is revealed in association with plots or lines 22-26. For instance, at line 24, linear shrinkage is seen to be about 30% for a temperature of 62.5° C. held for a ten minute duration. Curve 24 may be compared with curve 22 where shrinkage of about 36% is achieved in very short order where the temperature is retained at 65.5° C. Correspondingly, curve 26 shows a temperature of 59.5° C. and a very slow rate of shrinkage, higher levels thereof not being reached. Clinicians generally would prefer a shrinkage level on the order of 10% to 20% in dealing with skin laxity.

FIG. 3 reveals a schema representing the organization of skin. Shown generally at 28, the illustrated skin structure is one of two major skin classes of structure and functional properties representing thin, hairy (hirsute) skin which constitutes the great majority of the body's covering. This is as opposed to thick hairless (glabrous) skin from the surfaces of palms of hands, soles of feet and the like. In the figure, the outer epidermis layer 30 is shown generally extending over the dermis layer represented generally at 32. Dermis 32, in turn, completes the integument and is situated over an adjacent subcutaneous tissue layer (or hypodermis) represented generally at 34. Those involved in the instant subject matter typically refer to this adjacent subcutaneous layer 34 which has a substantial adipose tissue component as a “fat layer” or “fatty layer,” and this next adjacent subcutaneous tissue layer is also called the “hypodermis” by some artisans. The figure also reveals a hair follicle and an associated shaft of hair 36, vascular structures 37 feeding the dermis 32 and sweat glands 38. Not shown in FIG. 3 are a number of other components, including the cellular structure of the dermis, and the vascular tissues supplying the vascularized dermis and its overlying epidermis.

Epidermis 30 in general comprises an outer or surface layer, stratum corneum composed of flattened, cornified non-nucleated cells. This surface layer overlays a granular layer, stratum granulosum composed of flattened granular cells which, in turn, overlays a spinous layer, stratum spinosum composed of flattened polyhedral cells with short processes or spines and, finally, a basal layer, stratum basale, composed columnar cells arranged perpendicularly. For the type skin 28, the epidermis will exhibit a thickness from 0.07 to 0.15 mm. Heating implants described herein will be seen to be contactable with the dermis 32 at a location representing the interface between dermis 32 and next adjacent subcutaneous tissue or fat layer 34. The dermis in general comprises a papillary layer, subadjacent to the epidermis, and supplying mechanical support and metabolic maintenance of the overlying epidermis. The papillary layer of the dermis is shaped into a number of papillae that interdigitate with the basal layer of the epidermis, with the cells being densely interwoven with collagen fibers. The reticular layer of the dermis merges from the papillary layer, and possesses bundles of interlacing collagen fibers (as shown in FIG. 1) that are typically thicker than those in the papillary layer, forming a strong, deformable three dimensional lattice around the cells of the reticular dermis. Generally, the dermis is highly vascularized, especially as compared to the avascular epidermis. The dermis layer 32 will exhibit a thickness of from about 1.0 mm to about 4.0 mm.

For the purposes of the application, “intradermal” is defined as within the dermis layer of the skin itself. “Subcutaneous” has the common definition of being below the skin, i.e. near, but below the epidermis and dermis layers. “Subdermal” is defined as a location immediately interior to, or below the dermis, at the interface between the dermis and the next adjacent subcutaneous layer sometimes referred to as hypodermis. “Hypodermal” is defined literally as under the skin, and refers to an area of the body below the dermis, within the hypodermis, and is usually not considered to include the subadjacent muscle tissue. “Peridermal” is defined as in the general area of the dermis, whether intradermal, subdermal or hypodermal. Transdermal is defined in the art as “entering through the dermis or skin, as in administration of a drug applied to the skin in ointment or patch form,” i.e. transcutaneous. A topical administration as used herein is given its typical meaning of application at skin surface.

As noted, the thickness of the epidermis and dermis vary within a range of only a few millimeters. Thus subcutaneous adipose tissue is responsible in large part for the overall contours of the skin surface, and the appearance of the individual patient's facial features, for instance. The size of the adipose cells may vary substantially, depending on the amount of fat stored within the cells, and the volume of the adipose tissue of the hypodermis is a function of cell size rather than the number of cells. The cells of the subcutaneous adipose tissue, however, have only limited regenerative capability, such that once killed or removed, these cells are not typically replaced. Any treatment modality seeking to employ heat to shrink the collagen of the ECM of the skin, must account for the risk associated with damaging or destroying the subcutaneous adipose layer, with any such damage representing a large risk of negative aesthetic effects on the facial features of a patient.

In general, the structural features of the dermis are determined by a matrix of collagen fibers forming what is sometimes referred to as a “scaffold.” This scaffold, or matrix plays an important role in the treatment of skin laxity in that once shrunk, it must retain it's position or tensile strength long enough for new collagen evolved in the healing process to infiltrate the matrix. That process is referred to as “neocollagenesis.” Immediately after the collagen scaffold is heated and shrunk it is no longer vital because it has been exposed to a temperature evoking an irreversible denaturation. Where the scaffold retains adequate structural integrity in opposition to forces that would tend to pull it back to its original shape, a healing process requiring about four months will advantageously occur. During this period of time, neocollagenesis is occurring, along with the deposition and cross linking of a variety of other components of the ECM. In certain situations, collagen is susceptible to degradation by collagenase, whether native or exogenous.

Studies have been carried out wherein the mechanical properties of collagen as heated were measured as a function of the amount of shrinkage induced. The results of one study indicated that when the amount of linear shrinkage exceeds about 20%, the tensile strength of the collagen matrix or scaffold is reduced to a level that the contraction may not be maintained in the presence of other natural restorative forces present in tissue. Hence, with excessive shrinkage, the weakened collagen fibrils return from their now temporary contracted state to their original extended state, thereby eliminating any aesthetic benefit of attempted collagen shrinkage. The current opinion of some investigators is that shrinkage should not exceed about 25%.

One publication reporting upon such studies describes a seven-parameter logistic equation (sigmoidal function) modeling experimental data for shrinkage, S, in percent as a function of time, t, in minutes and temperature, T, in degrees centigrade. That equation may be expressed as follows:

$$S ( t , T ) = \frac{[ {a}_{0} ( T - 62 ) + {a}_{1} ] - {a}_{2}}{1 + {( \frac{t}{{a}_{3} {e}^{- a [ T - 62 ]}} )}^{( {a}_{4} ( T - 62 ) + {a}_{5} )}} + {a}_{2} \qquad ( 1 )$$

Equation (1) may, for instance, be utilized to carry out a parametric analysis relating treatment time and temperature with respect to preordained percentages of shrinkage. For example, where shrinkage cannot be observed by the clinician then a time interval of therapy may be computed on a preliminary basis. For further discourse with respect to collagen matrix shrinkage, temperature and treatment time, reference is made to the following publication:
16. Wall, et al., “Thermal Modification of Collagen” Journal of Shoulder and Elbow Surgery, 8:339-344 (1999).

At the commencement of studies leading to the instant discourse, it was contemplated that dermis would be heated by radiofrequency current passing between bipolar arranged electrodes located at the interface between dermis and the next subcutaneous tissue or fat layer. To protect that subcutaneous layer, the electrodes are supported upon a polymeric thermal barrier. That barrier support was to be formed of a polymeric resin such as polyetherimide available under the trade designation “Ultem” from the plastics division of General Electric Company of Pittsfield, Mass. Initially, testing of this approach was carried out ex vivo utilizing untreated pigskin harvested about 6-8 hours prior to experimentation. Such skin was available from a facility of the Bob Evans organization in Xenia, Ohio. To position the implant at the interface between dermis and fat layer, a blunt dissecting instrument was employed to form a heating channel, whereupon the implant was inserted over the instrument within that channel with its electrodes located for contact with dermis while the polymeric thermal barrier functioned to protect the fatty layer. It may be noted that such polymeric material is both thermally and electrically insulative. Following implant positioning, the instrument was removed.

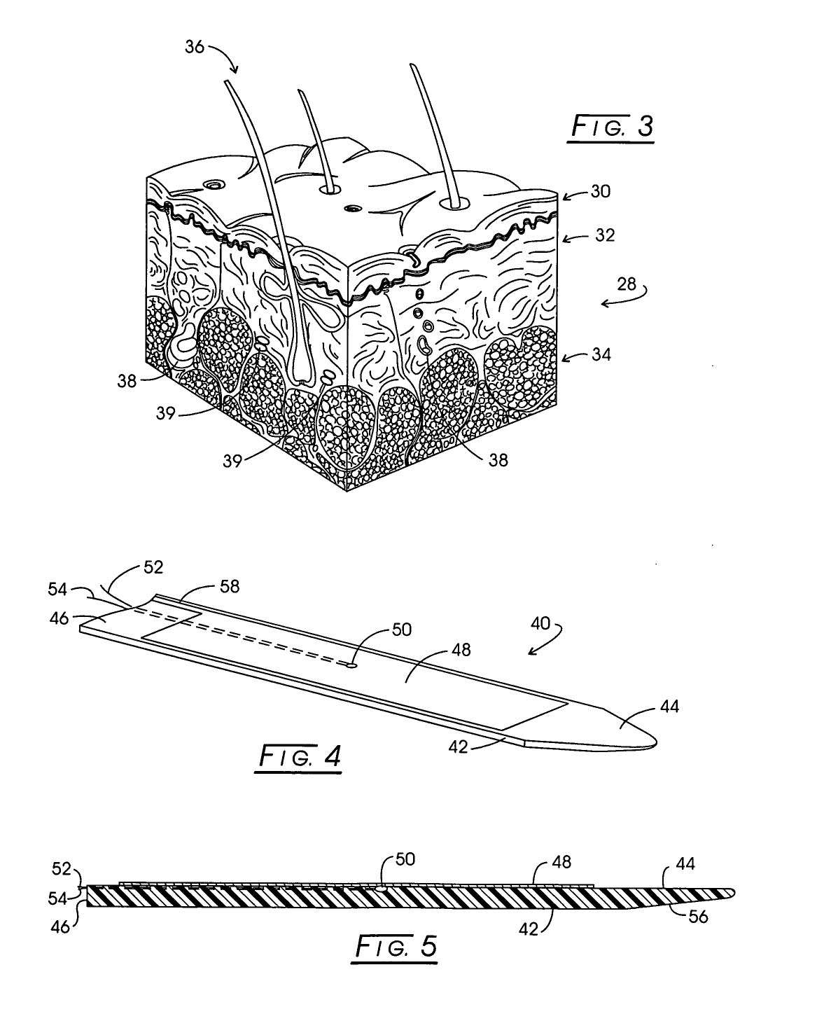

Looking to FIG. 4, an experimental implant is represented generally at 40. Implant 40 is configured with a polymeric electrically and thermally insulative support and barrier shown generally at 42 having a tapering leading end 44 and a trailing end 46. Thermal barrier 42 had a thickness of 0.037 inch and a width of 0.150 inch. Adhesively bonded to the support surface of barrier 42 is a platinum electrode 48. Electrode 48 has a thickness of 0.001 inch, a width of 0.150 inch and is 1.0 inch long. Looking additionally to FIG. 5, a thermocouple 50 is located in electrically insulative but thermally responsive relationship with the electrode 48. Electrically insulated leads 52 and 54 extend from operable connection with thermocouple 50 outwardly from the trailing end 46 of thermal barrier 42. FIG. 5 further reveals that the leading end 44 of thermal barrier 42 is upwardly tapered as at 56. Taper 56 tends to mechanically bias the implant toward contact with the dermis when inserted within a heating channel. An integrally formed lead to electrode 48 is seen at 58.

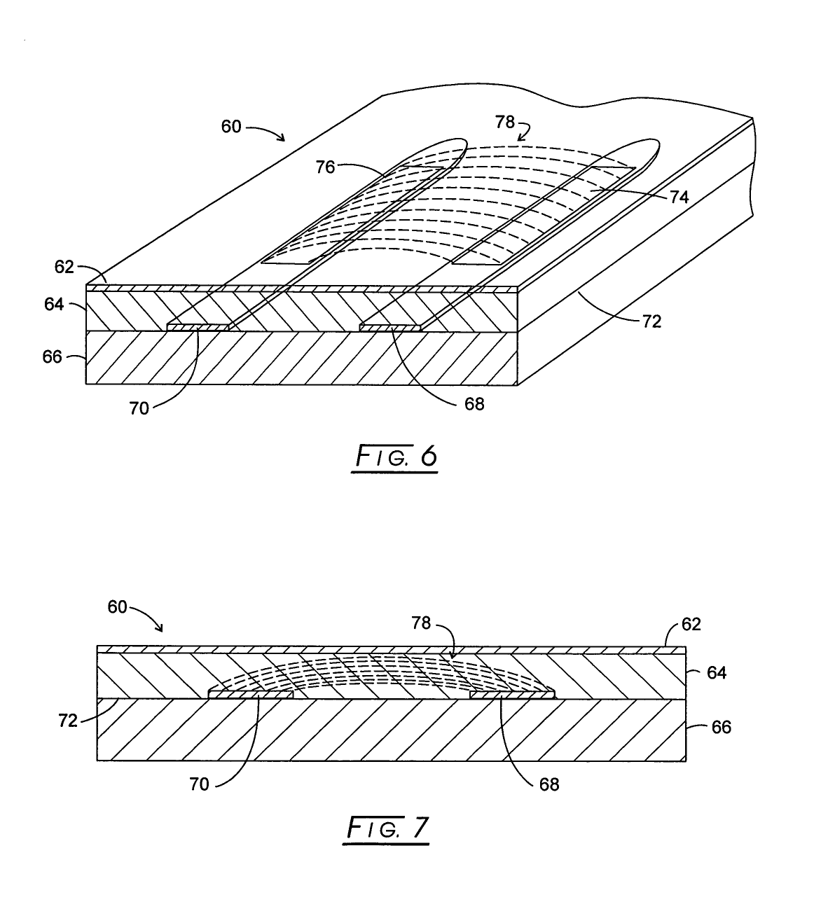

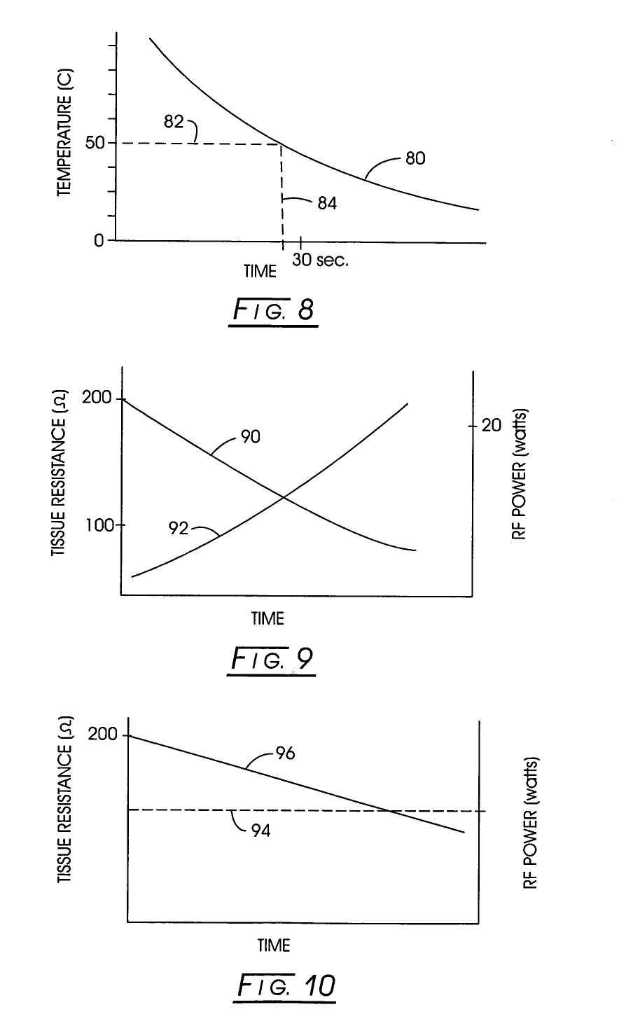

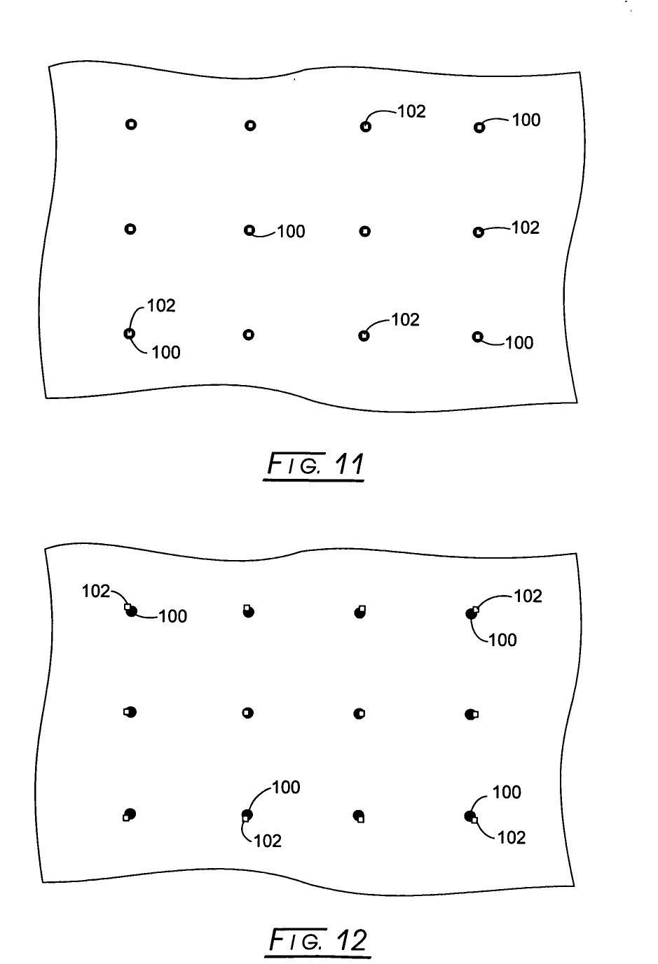

Turning to FIGS. 6 and 7, a schematic portrayal is provided of the ex vivo experimental set-up. In the figures, harvested pigskin is represented generally at 60 having an outer epidermis layer 62; a dermis layer 64; and a next subcutaneous tissue layer or fat layer 66. Two spaced apart and parallel implants 68 and 70 are located within heating channels at the interface 72 between dermis layer 64 and fat layer 66. Thus positioned, the identically dimensioned platinum electrodes shown respectively at 74 and 76 with respect to implants 68 and 70 in FIG. 6 were located in parallel adjacency. The implants 68 and 70 were spaced apart a distance of 15 mm center-to-center. This spacing is about twice that employed for implants configured for electrically resistive heater based approaches. With the arrangement shown, the electrodes 74 and 76 are contactable with the bottom of dermis layer 64. Radiofrequency energy was applied in bipolar fashion to electrodes 74 and 76 to generate a current flux path represented generally and schematically by dashed lines 78. Note that this current flux is represented as being confined to dermis layer 64. In this regard, it may be observed that the electrical conductivity exhibited at dermis layer 64 is about 5-10 times the electrical conductivity of the next adjacent fat layer 66. It was determined that to achieve significant collagen shrinkage it is necessary for the dermis to reach thermal transition temperatures of from about 62° C. to about 67° C. That temperature was found to be reachable in 50 to 60 seconds. Because of the spacing between electrodes 74 and 76, the current flux 78 creates a zone of heated dermis. That heat will commence to heat the fat layer 66 between the implants 68 and 70 by virtue of thermal conduction. Some of the heat which conducts into the fat layer 66 is carried away by the perfusion of blood flowing through the fat layer 66 and at the interface between the fat layer 66 and the dermis 64 which serves to limit the temperature rise on the fat layer 66. However, studies such as those carried out by Henriques and Moritz, indicate that tissue cells suffer irreversible cell death in accordance with a temperature and time relationship. Looking to FIG. 8, such a relationship is schematically depicted with respect to curve 80. As represented at dashed lines 82 and 84, for instance, at 50° C. it requires about 30 seconds of thermal dosage to create cell death. The shrinkage reactions resulting from the instant experiment show that requisite temperatures are not sustained for an adequate interval of treatment to create cell death phenomena at the subcutaneous tissue layer 66. For these earlier experiments, the epidermis 62 was cooled with blown air or mist.

See generally the following publication:
17. Henriques, F. C., Jr., Studies of Thermal Injury. V. “The Predictability and Significance of Thermally Induced Rate Processes Leading to Irreversible Epidermal Injury.” Arch. Path., 43, 489-502 (1947).

For instance, with the present system, the dermis may be held at about 50° C. for only 5-10 seconds. In some experimental runs, 20% shrinkage was observed within 50-60 seconds with 25 watts applied from an electrosurgical generator and about 25% shrinkage was observed, for example, at 60 seconds in some cases. In the course of these earlier experiments, it was known that the resistivity of dermis drops about 2% for every one degree centigrade temperature elevation. Conductivity is developed from the electrolyte within dermis tissue cells which is essentially normal saline. Initial studies utilizing an oscilloscope to measure power showed a resistance of the tissue commencing at about 200 ohms and as the procedure was carried out that resistance dropped to about 100 ohms.

Looking to FIG. 9, such a relationship of tissue resistance with time is portrayed at curve 90. Because the electrosurgical generator utilized exhibited a constant voltage supply, the power output of the generator tended to double as represented at power curve 92 which extends from a starting power output of about 10 watts and an elevation of that power with diminishing resistance is shown to reach about 20 watts. This elevation of power will cause the dermis to elevate in temperature, as it approaches 100° C. creating a steam layer with very large electrical impedance rendering the current flux path essentially non-conductive. Thus, consideration of utilizing a constant power output was made. Such constant power is represented at dashed line 94 in FIG. 10. Power level 94 is represented in conjunction with tissue resistance varying curve 96.

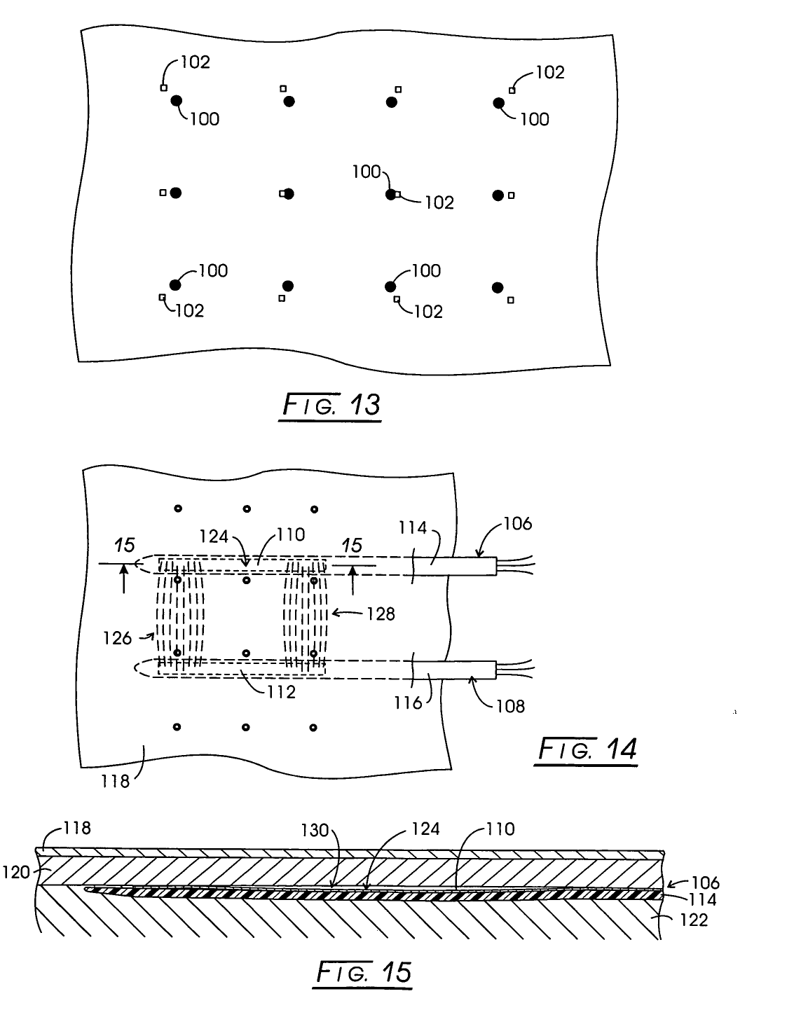

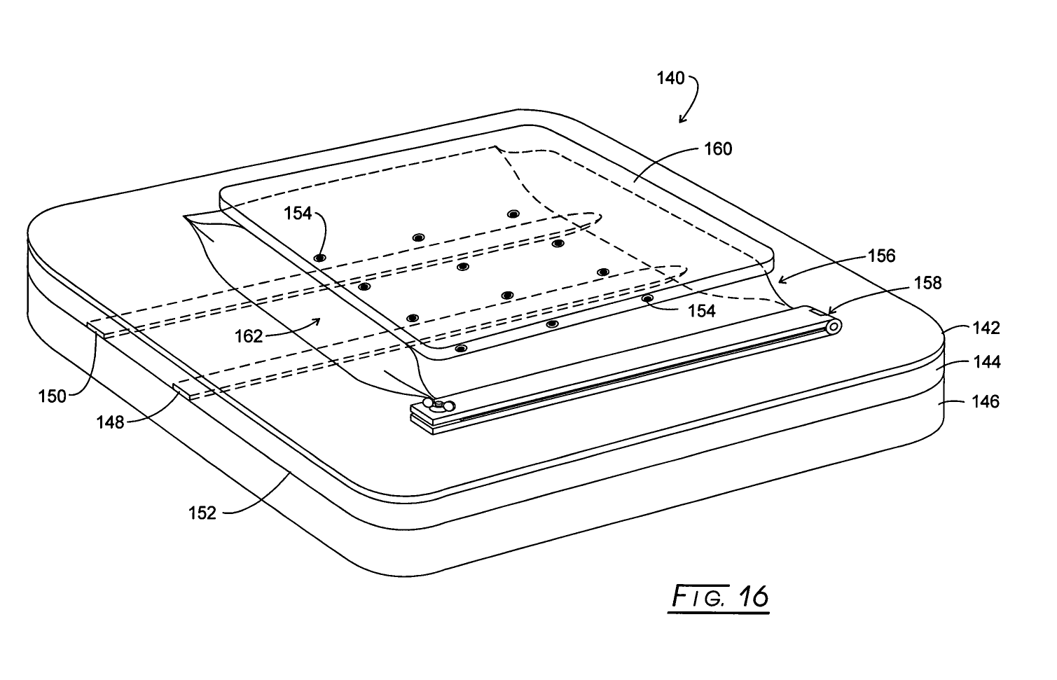

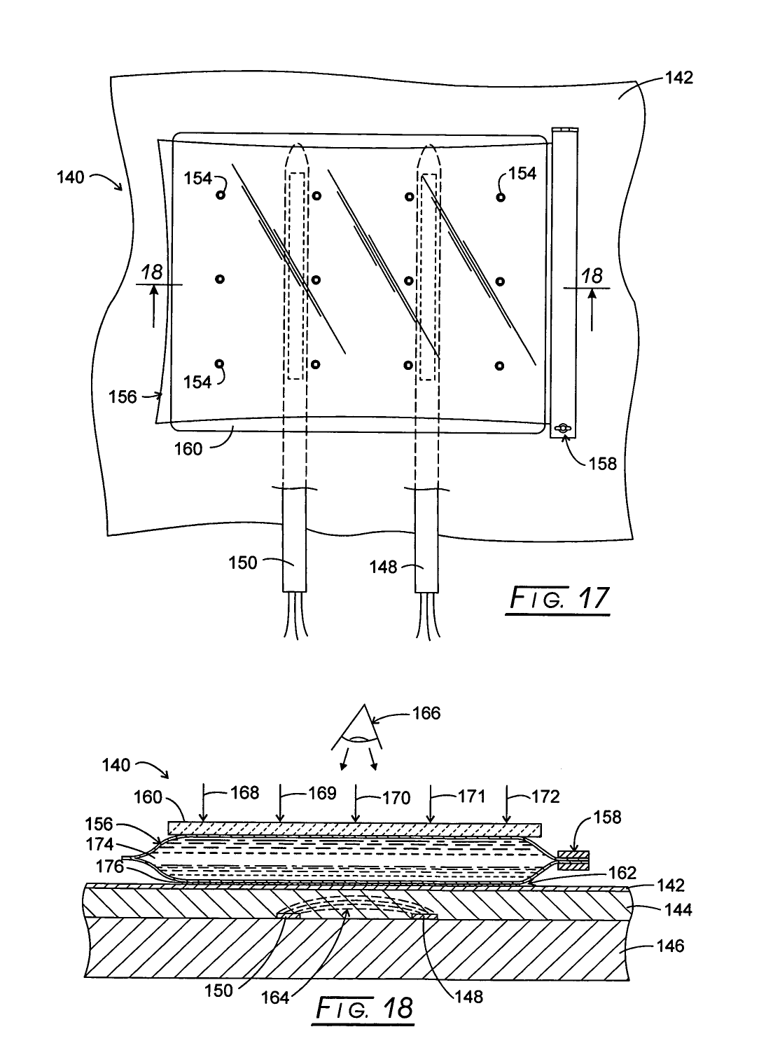

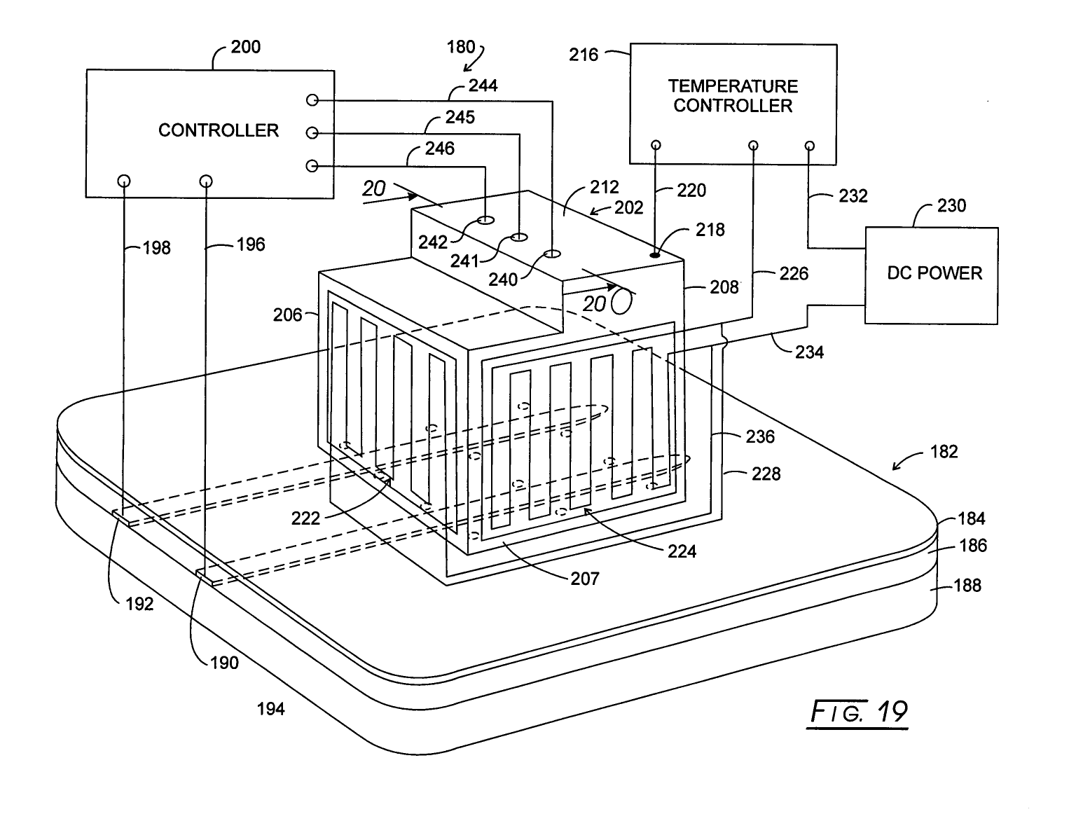

In the course of experimental runs utilizing platinum electrodes as at 48 (FIG. 4) it was observed that while significant collagen shrinkage was achieved within about a 60-90 second interval, for some runs the temperatures of the bipolar associated platinum electrodes were unusually separated in level. In this regard, for some runs one electrode (thermocouple) would exhibit a maximum temperature of 50° C. which is below the threshold or thermal transition temperatures for inducing shrinkage. It was observed that the thermal expansion coefficient of the polyetherimide thermal barrier 42 was 56×10⁻⁶ in/in/° C. and the corresponding thermal expansion coefficient for platinum was 9×10⁻⁶ in/in/° C. This meant that the thermal barrier would expand about 0.004 inch more than the platinum electrode at temperatures of about 70-80° C. This situation was born out by immersing the implant in water of about 80° C. to 85° C. The implant was seen to immediately curve. Such curving will always be concavely away from the lower surface of dermis. By contrast, immersion of a resistive heater implant formed with a very thin deposition of gold-plated copper on a substrate adhered to the polyetherimide material showed no warpage. This led to an awareness that performance of the system would be affected by a loss of uniform contact between the radiofrequency excited electrodes and the surface of dermis. Notwithstanding this potential phenomenon in vivo testing showed that the system achieved substantial shrinkage over a very short interval of about 60 seconds. In this regard, FIGS. 11, 12 and 13 are taken from an experimental run using paired implants as described at 40 in FIGS. 4 and 5. To quantify the extent of contraction or shrinkage a matrix-like pattern of dots or visible indicia were positioned initially at the skin region of interest. The initial position of those dots are represented by black circles certain of which are identified at 100. Digital imaging of the dots 100 was carried out and this initial position at time zero was digitally memorized as represented by the small white squares certain of which are identified at 102 which in FIG. 11 are centered within the dots 100. During the experiment, these squares 102 will digitally remain in position, however, as a consequence of heat induced dermis shrinkage, dots 100 will move with respect to squares 102. Looking to FIG. 12, the experiment is imaged at time 40 seconds. Note that the black dots 100 are relatively displaced from the stationary squares 102. Next, turning to FIG. 13, the relative positioning of dots 100 with respect to reference squares 102 are depicted at time 60 seconds and a condition under which power was turned-off at approximately 55 seconds. The resultant shrinkage is abundantly evident in the figure. The experimental run represented by FIGS. 11-13 was a test in a sequence of tests. Certain of those tests revealed the presence of thermal injury to the epidermis such as erythema and/or edema at regions of the epidermis above forward and rearward regions of the platinum electrodes as at 48 described in connection with FIG. 4. This led to a consideration of the disparate coefficients of thermal expansion of the electrode material with respect to the thermal barrier material. Looking to FIG. 14, paired implants as described at 40 are represented at 106 and 108. The platinum electrodes for these implants are shown respectively in phantom at 110 and 112. Electrodes 110 and 112 are adhesively mounted upon respective thermal barriers 114 and 116. Looking additionally to FIG. 15 the implant 106 reappears in sectional fashion as being located within a skin region incorporating epidermis 118, dermis 120 and next adjacent subcutaneous tissue or fat layer 122. Note that the implant is concavely bowed away from the dermis at its central region represented generally at 124. It was further observed that the dermis layer 120 itself contracted away from contact with the electrode as seen generally at region 130.

Returning to FIG. 14, this phenomena wherein the outward regions of the electrodes were the only regions contacting dermis resulted in a concentration of current flux between electrodes 110 and 112 as illustrated at dashed flux path representations shown generally at 126 and 128.

The situation observed with respect to FIGS. 14 and 15 lead to a consideration that tamponade or some form of slight pressure could be applied to the epidermis 118 to force a continuous contact between the upward surface of electrode 110 and dermis 120. As noted above, the epidermis as at 118 was cooled by blown air or mist and it was consistently found that the airflow rate could not adjust fast enough nor provide cooling rate adequate for radiofrequency heating methods because of the higher heating rates per unit area and associated fast transient heat-up rate of the skin surface. Often, the surface temperature of the skin would be over-cooled resulting in insufficient shrinkage or under-cooled resulting in burns at the skin surface.

In experiments both ex vivo and in vivo (pig) next carried out, a transparent plastic bag was filled with water and used to both cool and apply tamponade or slight pressure against the upper surface of the epidermis during radiofrequency heating of the dermis between parallel implants as described in conjunction with FIGS. 4 and 5. Such an arrangement is generally depicted in FIG. 16 at 140. In the figure epidermis is schematically represented at 142; dermis at 144 and next adjacent subcutaneous tissue layer or fat layer at 146. Two parallel implants carrying platinum electrodes are represented in phantom at 148 and 150 located at the interface 152 between dermis 144 and fat layer 146. Dot indicia, certain of which are represented at 154 were located in a matrix format at the surface of epidermis 142: A water-filled plastic transparent bag represented generally at 156 was filled with water and closed using a clamp fixture 158. To apply tamponade, a transparent sheet of glass 160 was positioned over the upper surface of bag 156. The inward or contact surface of bag 156 as shown in general at 162 thus was positioned against the surface of epidermis 142 and functioned to apply a small amount of pressure. Experiments were run with liquids of different temperature within bag 156. For instance, ice water did not work and what was contemplated was a form of heat sinking at temperatures near body temperature which maintained the surface of the skin at or slightly above about 30° C. and prohibiting any skin surface temperature elevation above 37° C. The setup 140 was employed with a heat transferring lubricant between contact surface 162 and the surface epidermis 142. It was found that a coating of water or glycerol functioned both as a lubricant permitting the skin surface to shrink during treatment and as a heat transfer medium to the liquid in the bag or container 156 which was required to perform as a heat sink. Water and glycerol exhibit a high thermal conductivity to provide for good heat transfer across the interface between the bag 156 and epidermis 142.

Looking additionally to FIGS. 17 and 18, the setup 140 is reproduced in a top view and a sectional view. In FIG. 18, a current flux path is represented generally at 164 flowing between the platinum electrodes of implants 148 and 150. During the procedure, the indicia as at 154 (FIG. 17) could be observed as represented at the eye station 166. Slight pressure is applied through the glass plate 160 as represented by arrows 168-172. Alternatively, the weight of the water filled bag will provide sufficient tamponade if the bag is at least 1.5 inches thick. Water is schematically represented at 174 within bag 156. Additionally, a layer of heat transferring and lubricating water is shown at 176 intermediate the contact surface 162 of bag 156 and the surface of epidermis 142. With this arrangement, for the water-filled plastic bag 156 to perform adequately as a heat sink it was necessary to agitate the water 174 at least at it's adjacency with the contact surface 162. Initially, a conventional magnetic stirring apparatus was utilized for this purpose. With such an arrangement, skin surface temperatures were maintained between about 38° C. and about 40° C.

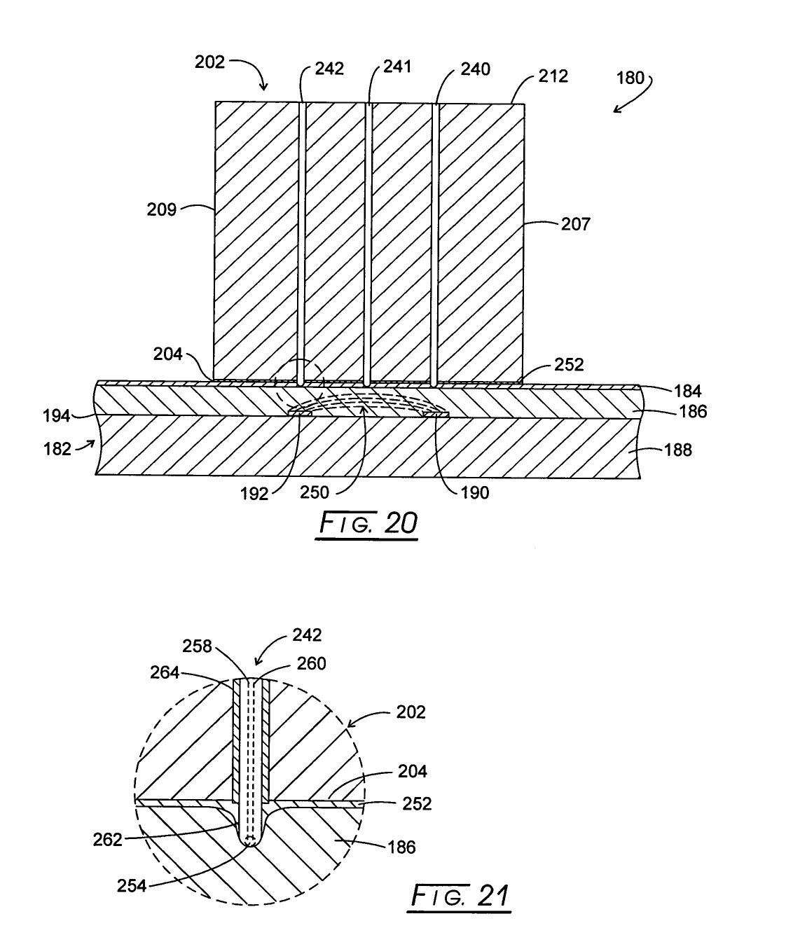

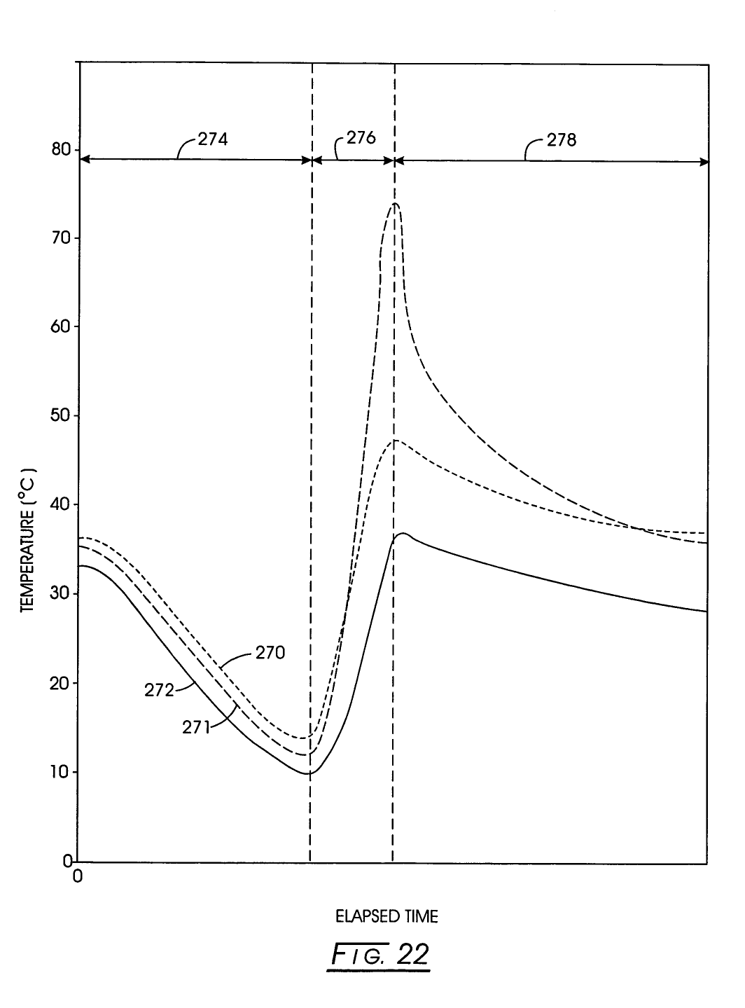

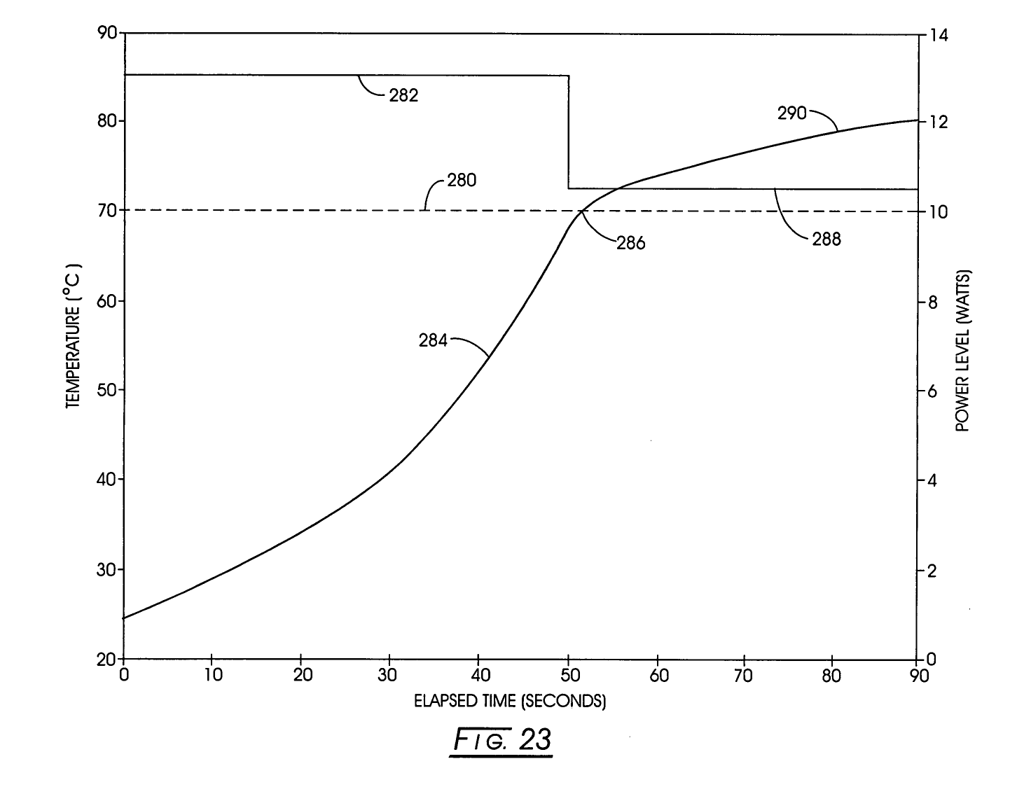

Experimentation was also carried out utilizing an instrumented and heated aluminum heat sink. Referring to FIGS. 19-21, such a setup is represented generally at 180. As before, the experiments were carried out both ex vivo and in vivo in conjunction with skin (pig) as represented in general at 182. In the figures schematically represented are epidermis 184; dermis 186; and next adjacent subcutaneous tissue or fat layer 188. Implants as described in connection with FIGS. 4 and 5 are shown at 190 and 192 located at the interface 194 between dermis 186 and fat layer 188. Implants 190 and 192 were spaced apart 15 mm center-to-center and arranged in parallel adjacency. RF excitation to the platinum electrodes of implants 190 and 192 is represented at respective lines 196 and 198 extending from a controller function represented at controller block 200. Resting upon the epidermis layer 184 is a block-shaped aluminum heat sink represented generally at 202. Heat sink 202 was dimensioned with a contact surface seen in FIG. 20 at 204 which is defined by 4 two inch wide sides 206-209 (FIGS. 19, 20) and having a height at top portion 212 of 2½ inches. Sides 206 and 207 were heated by “copper on Kapton” (polyamide) resistance heaters which were controlled from a commercial temperature controller represented at block 216. Controller 216 monitored the temperature of heat sink 202 at a thermocouple 218 as represented at line 220. Controlled d.c. power was supplied to the resistance heating functions identified generally at 222 and 224 as represented by circuit lines 226 and 228 controller 216. Control to the d.c. power function represented at block 230 from controller 216 is at line 232. Power input to the resistive heating functions 222 and 224 is represented extending from power function 230 with lines 234 and 236. Three bores 240-242 are seen extending through the heat sink 202, each of these bores carries a seed thermocouple, each exhibiting a small outside diameter. The outputs of the thermocouples to the controller function with respect to bores 240-242 are represented at lines 244-246. Aluminum heat sink 202 was electrically insulated to avoid interference with R.F. current flux by being clear hard anodized. FIG. 20 reveals implants 190 and 192 as well as a current flux represented generally at 250 extending between the platinum electrodes thereof within dermis layer 186. A layer of water represented at 252, as before, provided lubrication and improved thermal transfer between the skin surface and heat sink 202. FIG. 20 further reveals that the spacing between bores 240-242 corresponded with the center-to-center spacing of implants 190 and 192, i.e., 15 mm. Additionally, bore 241 is spaced evenly between bores 240 and 242. Looking additionally to FIG. 21, bore 242 reappears in enlarged form. Within that bore was a 0.020 inch outside diameter (OD) stainless steel sheath 262, the bottom portion of which carries the very small seed thermocouple as revealed at 254. Paired leads 258 and 260 extend from the thermocouple 254 providing the function represented at line 245 in FIG. 19. Stainless steel sheath 262, within the body of heat sink 202 is wrapped with a thermal and electrically insulating shrink wrap tubing 264 having a thickness of 0.002 inch. It was deemed desirable that thermocouples as at 254 be supported to measure the temperature at the epidermis surface as opposed to being influenced by the temperature of the heat sink 202. Accordingly, the stainless steel sheaths as at 262 extended below contact surface 204 a distance of 0.020 inch such that each thermocouple was located within a slight depression within the epidermis layer 186. The weight of the heat sink 202 itself provided requisite tamponade or pressure. In this regard, the heat sink exhibited a weight of 0.875 pounds to provide a pressure of about 0.219 pounds per square inch. The temperature controller 216 was found to maintain the temperature of heat sink 202 at 40° C. plus or minus 0.5° C. Use of this form of heat sink further demonstrated that the layer of water 252 improved the heat sink function. Maximum skin surface temperature as measured with these three thermocouples, one of which has been illustrated at 254 remained between about 42° C. and 43° C. The temperatures never rose above 43° C. for any of the experimental runs and the temperatures recorded with the thermocouples remained within 1° C. of each other. The hottest skin temperature measured was by the thermocouple in bore 241 which was centered between implants 190 and 192. Substantial skin contraction was achieved with the radiofrequency powered electrodes in very short time intervals, for example, between about 60 seconds and about 90 seconds. By lowering the power levels derived from an associated electrosurgical generator the treatment interval can be expanded. However, in view of the initial quite rapid achievement of requisite shrinkage, to protect the next adjacent subcutaneous layer it was contemplated that a pre-cooling of that layer prior to therapy may be beneficial. Additionally, the heat sink function can be continued for an interval following the therapy interval. That procedure can be computationally analyzed. Referring to FIG. 22, such a computation is graphically illustrated. In the figure, temperature is plotted against arbitrary time units in conjunction with the temperature of the next adjacent subcutaneous or fat layer; the temperature of the electrodes; and the temperature of the skin layer combining epidermis and dermis. Such curves are represented respectively at 270-271. During a pre-cooling interval represented at horizontal arrow 274 these three components represented at curves 270-272 show a drop in temperature from body temperature (about 37° C.) to three levels representing at about 12° C.-15° C. When that pre-cooling is completed, as represented at horizontal arrow 276 therapy takes place with a marked elevation in temperature of the electrodes and skin as represented at curve 271 and 272 and an elevation of the subcutaneous fat layer as represented at curve 270. In general, heat at the next adjacent subcutaneous layer will be of a thermally conductive nature occasioned by a heating of the dermis between the two electrodes as discussed in connection with FIGS. 19-21. While curve 271 shows that the electrodes elevated in temperature to about 75° C. at the end of therapy, the subcutaneous fat layer remains at a level above about 45° C. In general, vascularity or blood perfusion within that layer will produce a natural heat controlling effect. By maintaining the heat sink in position following therapy, as represented at arrow 278, the curves 270-272 are seen to normalize toward body temperature.

One approach to control over the electrode-based heating process has been described in conjunction with utilization of a constant power source. Another approach is to monitor temperature during the therapy interval and step down the amount of power applied to the electrodes as setpoint or target temperature is approached or reached. Looking to FIG. 23, monitored temperature of an electrode is plotted with respect to elapsed therapy time in seconds as well as with respect to power level. In the figure, setpoint or target temperature is represented at horizontal dashed line 280, while an initially applied power level of about 13 watts is represented at power level segment 282. Monitored electrode temperature is initially represented at curve portion 284 which shows a rapid rise in temperature for an initial 50 seconds elapsed time. As the setpoint temperature at level 280 is reached at curve position 286 a stage 2 power level represented at curve portion 288 is derived. For the instant example, curve portion 288 represents a 20% step-down in power which occurs when either electrode reaches the target temperature. For example, that stage 2 power level at curve portion 288 may be about 10.5 watts. As this occurs, electrode temperature curve portion 290 rate of temperature rise drops significantly. Alternately, bipolar excitation of paired electrodes may be undertaken at a fixed applied power level (or current level) until the electrode temperatures reach a first setpoint at which time the power (or current) is reduced to some fraction of the initial power (or current), e.g., to 50% until the final temperature setpoint is attained, which may be maintained for an additional “soaking interval”.

The principal structure of implants configured according to the invention is one wherein a thermally and electrically insulative support is provided which performs as a thermal barrier. Such support is configured, for instance, with the earlier-described polyetheramide, “Ultem”. That thermal barrier and support is combined with a flexible circuit arrangement formed of the earlier-described “Kapton” with, for one embodiment, gold-plated copper electrodes on one surface and rectangular spiral (serpentine) resistor segments on the opposite side aligned with the one or more electrodes. Those resistor segments also are formed of gold-plated copper and it is that side of the flexible circuit which is adhered to a surface of the thermal barrier which is arbitrarily described as a “support surface”. In similar fashion, the opposite surface of the thermal barrier is arbitrarily described as an “insulative surface”. To determine electrode temperature, the resistance exhibited by the resistor segment which is aligned with the electrode is sampled and correlated with temperature. These resistor segments as well as associated electrodes also can perform as a “thermal spreader” functioning to promote uniformity of temperature extending into dermis. A “four-point” printed circuit lead assembly is employed to gather resistance and thus temperature data in a manner immune from the impedance characteristics of lengthy cables leading from the implant to a controller.

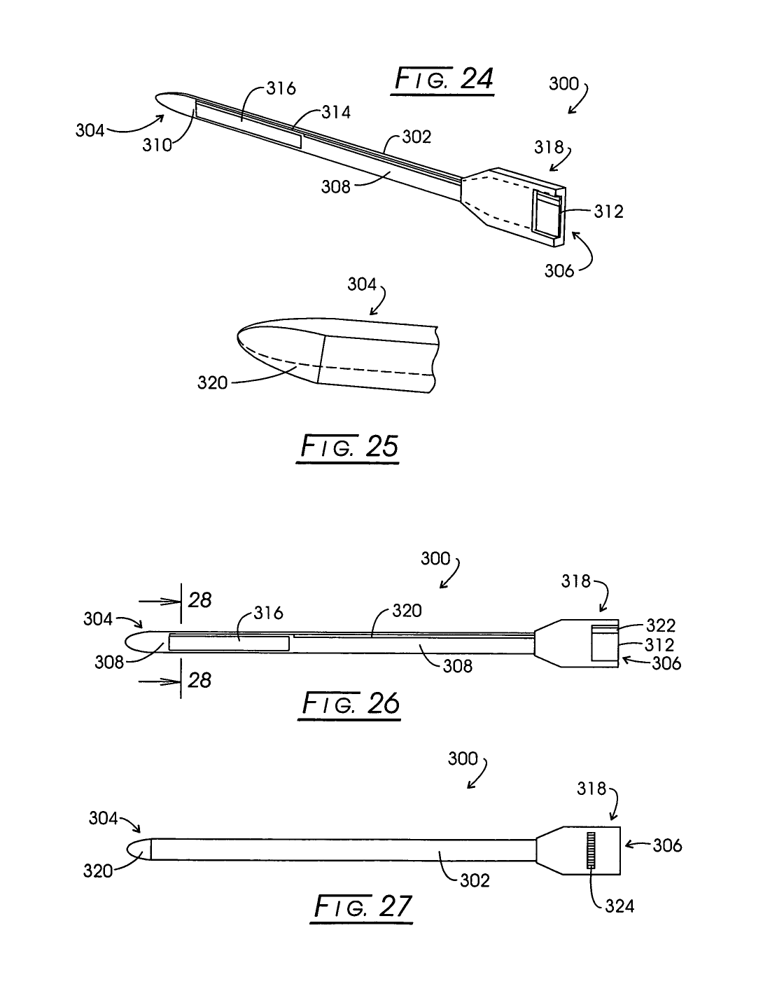

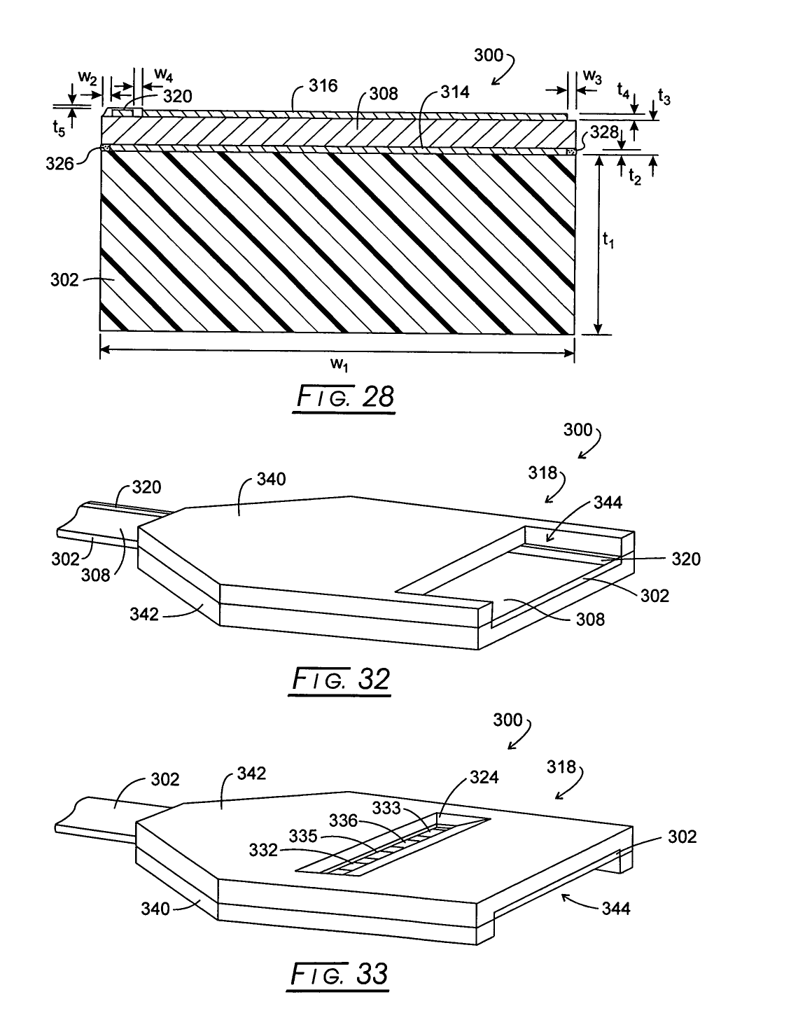

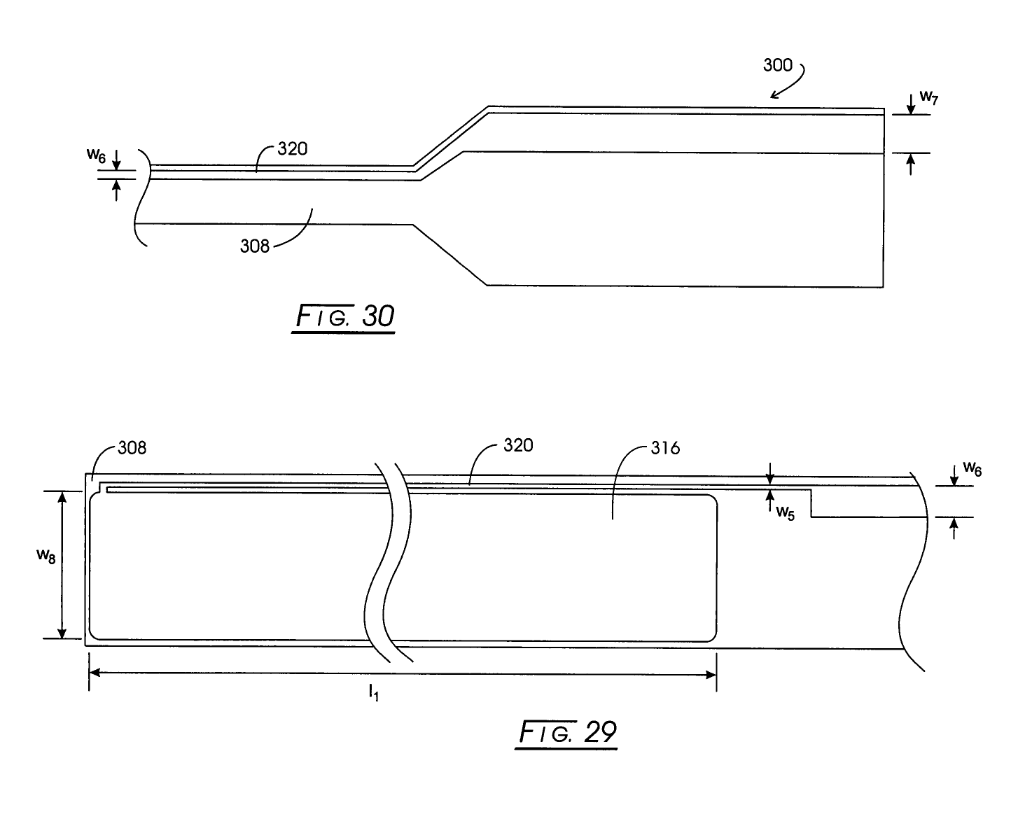

The initial implant embodiment described herein is a single channel or single electrode type and is illustrated in connection with FIGS. 24 through 33. Its structural configuration in terms of component layers, thicknesses, electrical insulation and cable connector guides will be found to be essentially repeated in the embodiments for multiple channel or multiple electrode implants. Referring to FIG. 24, a single electrode implant is represented generally at 300. Implant 300 is configured with a thermally insulative support functioning as a thermal barrier which is illustrated generally as a layer 302 which extends from a leading end represented generally at 304 to a trailing end represented generally at 306. Adhesively adhered to the support surface of thermal barrier 302 is a flexible circuit comprised of the earlier-described “Kapton” substrate represented at layer 308. Layer 308 is adhesively secured to the support surface of thermal barrier 302 and extends from a forward end 310 just behind thermal barrier leading end 304 to a rearward end 312 coincident with trailing end 306. The inner surface of substrate 308 supports a rectangular spiral (serpentine) resistor represented as a layer 314 which functions as a temperature measurement device. The opposite or outer surface of substrate 308 supports a gold-plated copper electrode 316 which is aligned with the rectangular spiral resistor. Substrate 308 and its associated thermal barrier 302 extend and expand in width to a polymeric connector guide represented generally at 318. Looking momentarily to FIG. 25, it may be observed that leading end 304 of the thermal barrier 302 is slanted forwardly to an extent effective to provide a mechanical bias toward dermis when the implant is inserted within a heating channel. That slanted region is shown at 320.

Looking to FIG. 26, a copper-plated gold trace 320 functioning as an electrical lead is shown extending and broadening to provide a contact 322 within the connector guide 318.

Referring to FIG. 27, a bottom view of the implant 300 is presented showing a side arbitrarily designated as an “insulative” surface of barrier 302. Note that connector guide 318 is configured with a rectangular opening 324 which functions to provide cable access to leads extending from the noted temperature-sensing resistor. Not seen in FIGS. 24-27 is an electrically insulative coverlay which functions to electrically insulate lead 320 as it extends to the forward end electrode 316.

Looking to FIG. 28, a sectional view of the implant 300 is presented. The polyetheramide thermal barrier 302 reappears with the same numeration and is identified as having a thickness, t₁. Thickness t₁ will have a value from about 0.02 inch to about 0.08 inch and is typically 0.037 inch. A rectangular spiral resistor shown as a layer of region 314 will exhibit a thickness within a range of about 0.0005 inch to about 0.005 inch. The latter thickness permits the resistor to function additionally as providing the noted thermal spreading function. An adhesive layer (not shown), for example, provided as a medical grade epoxy material is provided between the lower side of resistor 314 and the support side of thermal barrier 302. That adhesive layer will exhibit a thickness of from about 0.002 inch to about 0.005 inch. A certain amount of this adhesive will function to seal the resistor region 314 as represented at 326 and 328. The widthwise extent of adhesive components 326 and 328 is 0.005 inch. The flexible circuit substrate 308 as formed of the earlier-described “Kapton” will exhibit a thickness, t₃, of 0.001 inch and the electrode 316 which is again a gold-plated copper layer may exhibit a thickness t₄, in a range of about 0.0003 inch to about 0.005 inch. However, investigation has revealed that the electrodes as at 316 may also perform the function of thermal spreading. This beneficial effect is realized by enhancing their copper thickness, for example, to within a range from about 0.005 inch to about 0.020 inch. For instance, a thickness of 0.0056 inch has been found to be convenient inasmuch as it corresponds with conventional “4-ounce” copper. The gold-plated copper lead traces as at 320 are electrically insulated with a coverlay which is conformal but has a thickness, t₅, of about 0.001 inch.

Now considering the widths associated with the implant 300, the width of the thermal barrier 302, w₁, is 0.120 inch. The offsetting of lead 320 from the edge of the implant, w₂, was determined to be 0.005 inch; while the corresponding offset of the electrodes 316 from the edge of the “Kapton” surface, w₃ is also 0.005 inch. Finally, the offset of the lead 320 from electrode 316, w₄, was established as 0.003 inch.

Looking to FIG. 29, an enlarged view of electrode 316 and its associated lead 320 is presented. The width, w₅, of lead trace component 320 as it resides in adjacency with electrode 316 is about 0.005 inch. That width is increased rearwardly of electrode 316 as represented at, w₆, the lead trace width increases, for example, to about 0.032 inch. Finally, as represented in FIG. 30, at the rearward end of the implant 300, the lead trace width, w₇, increases to about 0.60 inch to facilitate contact with controller cabling. Returning to FIG. 29, the electrode 316 will have a length, l₁, of 0.6 to 1.0 inch and a nominal width, w₈, of 0.092 inch.

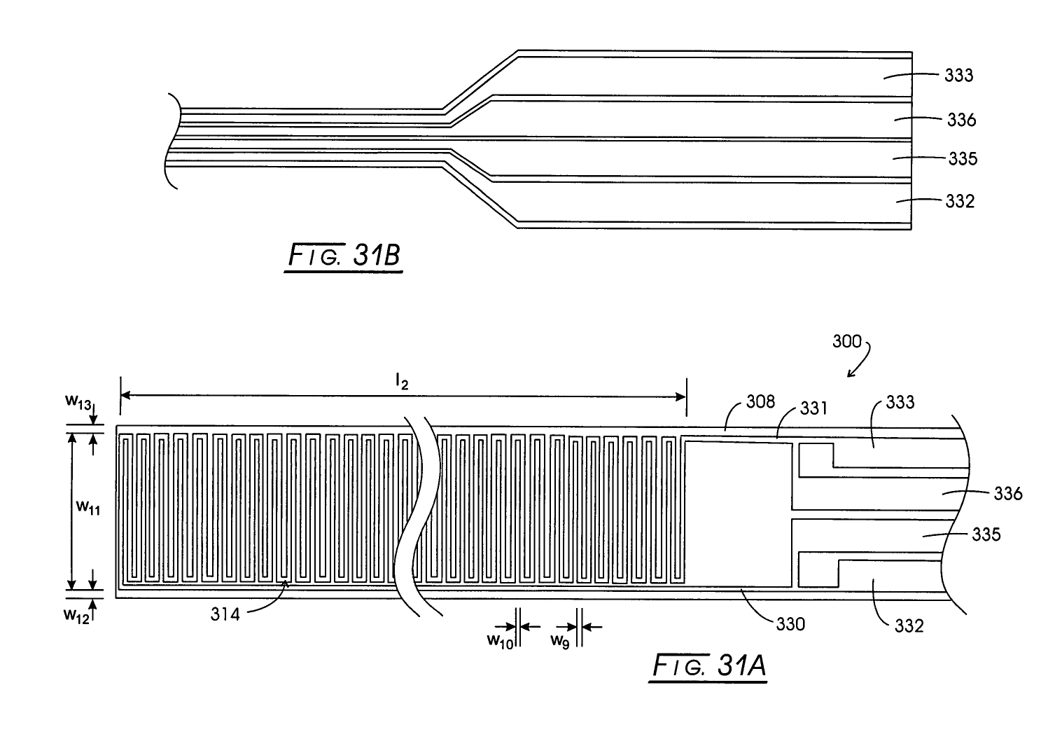

Turning to FIG. 31A, the arbitrarily designated inner surface of “Kapton” layer or substrate 308 is illustrated in enlarged detail. Temperature sensing resistor segment 314 is shown to have a rectangular serpentine or spiral configuration with a length, l₂, of 0.6 to 1 inch and is aligned with electrode 316 (FIG. 29) such that it is in thermal exchange association therewith. The trace width, w₉, of segment 314 is 0.003 inch and the spacing between trace lengths, w₁₀, also is 0.003 inch. The width of the segment 314, w₁₁, is 0.086 inch and the resistor segment is offset from the edges of the “Kapton” layer 308 distances, w₁₂ and w₁₃, which are 0.005 inch. Resistor segments as at 314, in general, are formed of a metal exhibiting a temperature coefficient of resistance greater than about 2,000 ppm/° C. Two lead traces of width, w₉, extend to respective source current input leads 332 and 333 of a four-point electrical connection which further includes voltage sensing taps or sensor leads 335 and 336. These leads extend to and are enlarged at the rearward end of the substrate as seen in FIG. 31B. As indicated above, through the utilization of the four-point approach involving leads 332-336 the resistance of resistor segment 314 may be measured or evaluated in a setting immune from the impedance characteristics of an associated cable.

Now looking to FIGS. 32 and 33, the connector guide or cable connector guide 318 for the single channel implant 300 is revealed at an enhanced level of detail. Such a guide is illustrated later herein in exploded fashion in conjunction with a multi-channel implant. In FIG. 32, guide 318 is seen to be fashioned of two interlocking components formed of white medical-grade polycarbonate and identified at 340 and 342. Component 340 is molded with a lead accessing notch represented generally at 344 which functions to expose the single lead trace 320. In FIG. 33, opening 324 is shown exposing four-point leads 332-336.

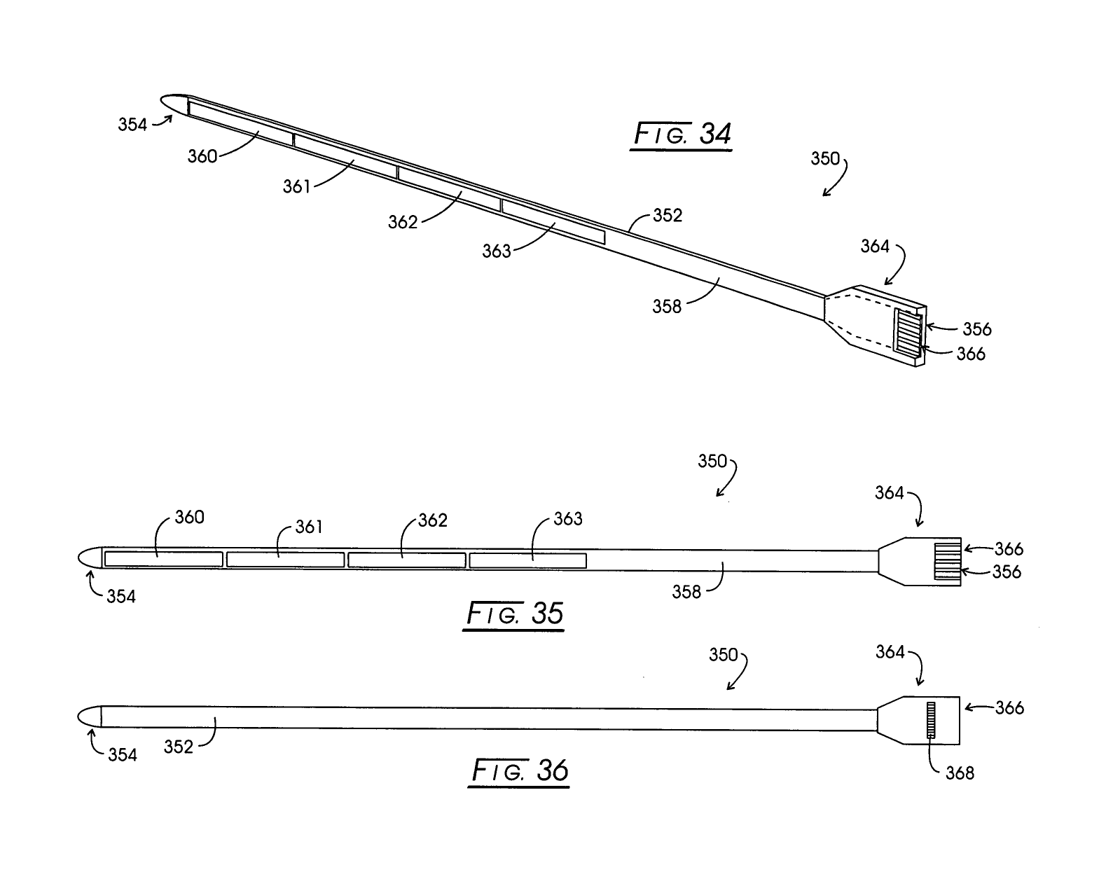

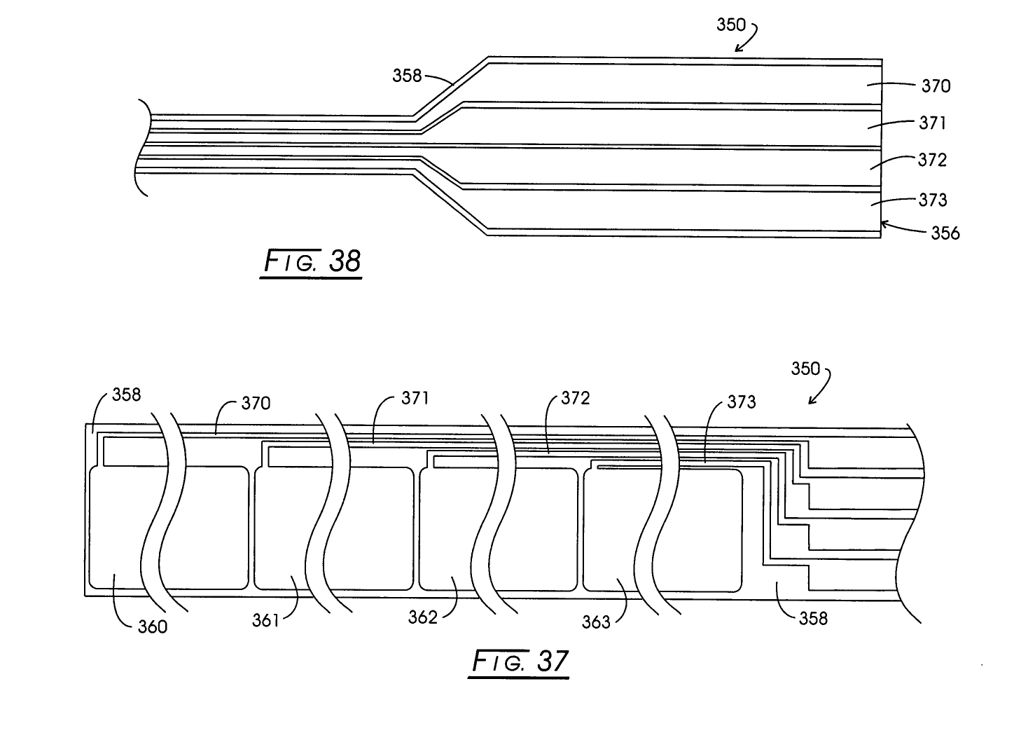

FIGS. 34-44 illustrate an implant having more than one electrode and an associated temperature-sensing resistor segment. In particular, the embodiment illustrated contains four electrodes and associated resistor segments or channels. With the exception of greater length, the implant dimensions heretofore discussed remain the same for these elongated embodiments. Referring to FIG. 34, a four-channel or four-electrode implant is represented generally at 350 in perspective fashion. As before, implant 350 is formed with a polyetheramide thermal barrier and support 352 having an arbitrarily designated insulative surface and oppositely disposed support surface. On that support surface there is adhered a flexible circuit formed with a polyamide substrate such as “Kapton” which, in turn, supports a sequence of four electrodes at its outer surface and a corresponding sequence of four resistor segments at its inner surface. Leads to the electrodes are electrically insulated with a coverlay. In the figure, the thermal barrier 352 is seen to extend from a leading end represented generally at 354 and a trailing end represented generally at 356. The leading end 354 is configured as described above in connection with FIG. 25, an arrangement normally mechanically biasing the implant 350 toward dermis when it is inserted within a heating channel. Flex-circuit polymeric substrate (“Kapton”) is represented at 358 supporting 4 one inch long rectangular gold-plated copper electrodes 360-363 along its active length and both the substrate and the thermal barrier extend to a polymeric connector guide represented generally at 364 having a notch or opening represented generally at 366 exposing leads extending to the electrodes 360-363. FIG. 35 is a top view of implant 350, while FIG. 36 is a bottom view showing the thermal barrier 352 and the opening extending therethrough as well as guide 364 as at 368 which permits access to the inward side of the flexible circuit substrate 358 and the leads thereon extending from four resistor segments. Referring to FIG. 37, an enlarged and broken away view of the electrode supporting active region of flexible circuit 350 is revealed. The outer surface of the flexible circuit substrate 358 is seen to support the generally rectangular gold-plated copper electrodes 360-363 and in addition a sequence of four gold-plated copper lead traces shown respectively at 370-373. Looking additionally to FIG. 38, these lead traces are seen extending to the trailing end 356 of the circuit support and thermal barrier 352. Such traces are electrically insulated with a coverlay where contactable with tissue.

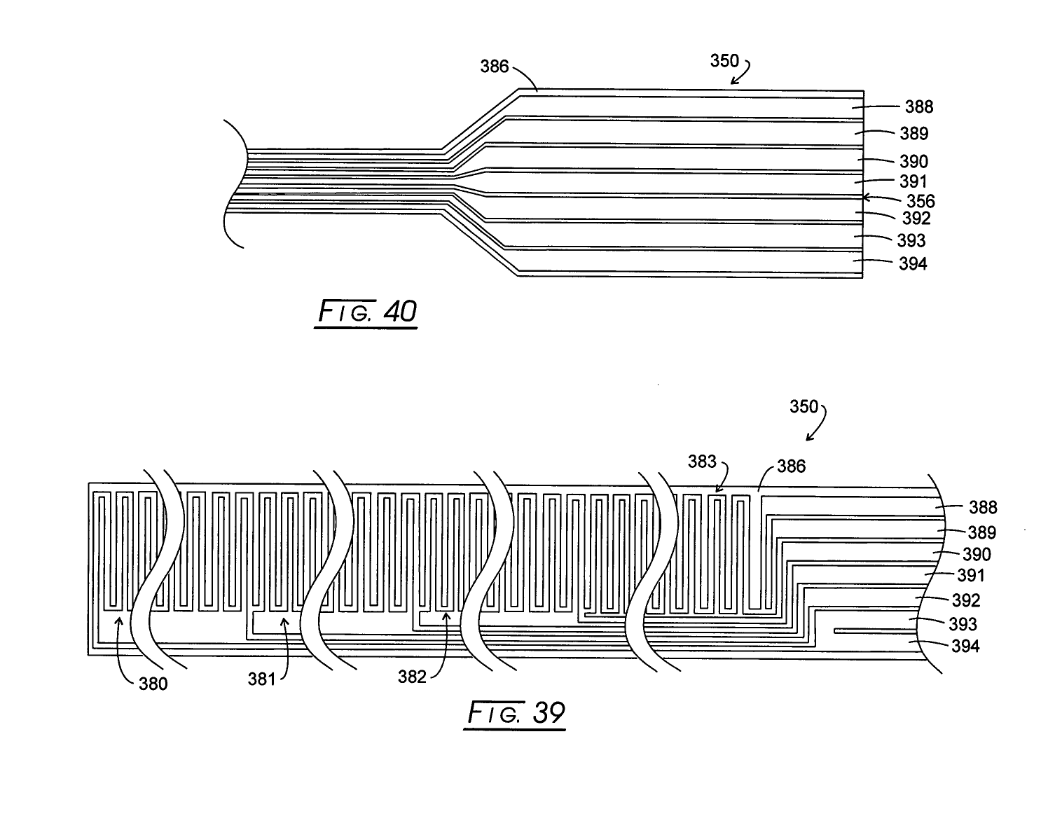

Looking to FIG. 39, an enlarged broken away view of the inward side of the flexible circuit substrate (“Kapton”) shows it to be supporting four gold-plated copper resistor segments 380-383 at inward surface 386. Segments 380-383 are aligned with corresponding respective electrodes 360-363 such that they are in thermal transfer relationship therewith to evaluate the temperature of the electrodes. These four sensing resistor segments are addressed by lead traces 388-393 which are arranged to provide a four-point interconnection. In this regard, leads 388 and 394 provide a low level d.c. source current, while leads 389-393 serve to provide a temperature sensor output. Referring to FIG. 40, leads 388-394 reappear at the trailing edge of the flexible circuit substrate.

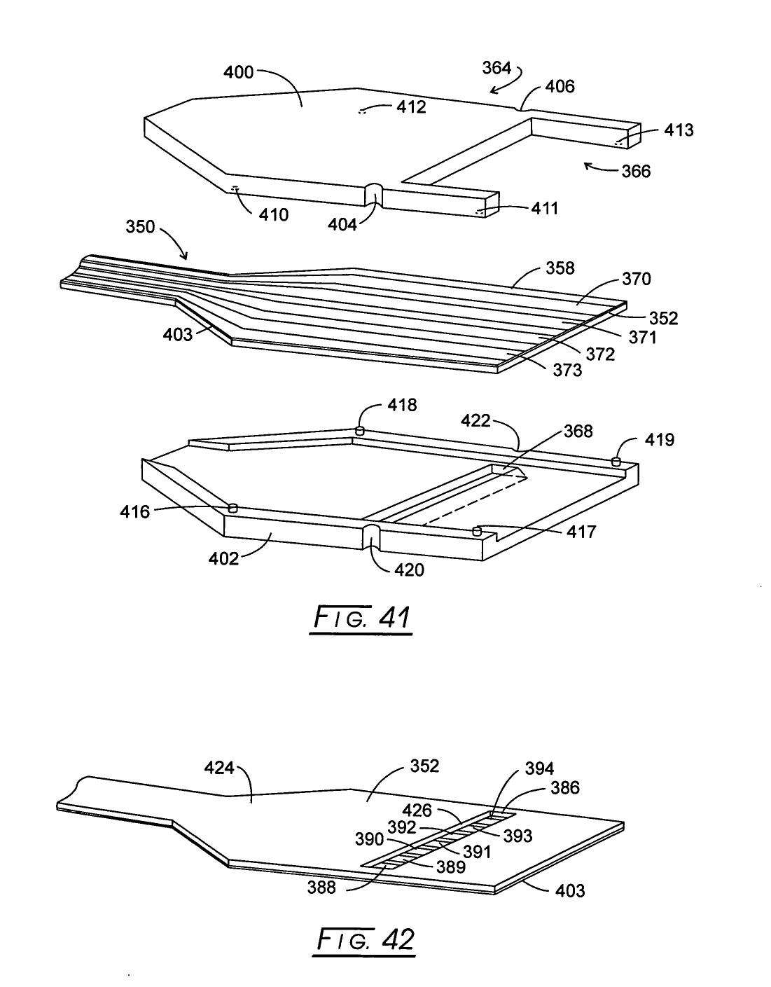

Referring to FIG. 41, an exploded view of the medical-grade polycarbonate connector guide 364 is provided. Guide 364 is configured in somewhat clamshell fashion being formed of two connector guide components 400 and 402. Components 400 and 402 are shown positioned above and below the trailing end region of implant 350. In this regard, flat support and thermal barrier 352 is observable in combination with the flexible circuit now identified as a layer 403. Component 400 is seen to incorporate earlier-described notch 366 as well as oppositely disposed detents 404 and 406 which are provided to assure a secure connection with a cable connector. Note, additionally, that component 400 is formed with upwardly depending cylindrical pin-receiver holes represented in phantom at 410-413. Component 402 incorporates window or opening 368 and four upstanding cylindrical alignment pins 416-419 which are configured to engage respective pin receiver holes 410-413 in a snap-together arrangement. Detents 420 and 422 correspond with respective detents 404 and 406.

Looking additionally to FIG. 42, the arbitrarily designated insulative surface 424 of thermal barrier 352 is depicted. Barrier 352 is configured with a rectangular window or opening 426 to expose the trace leads 388-394 located on the inner surface 386 of flexible circuit 403.

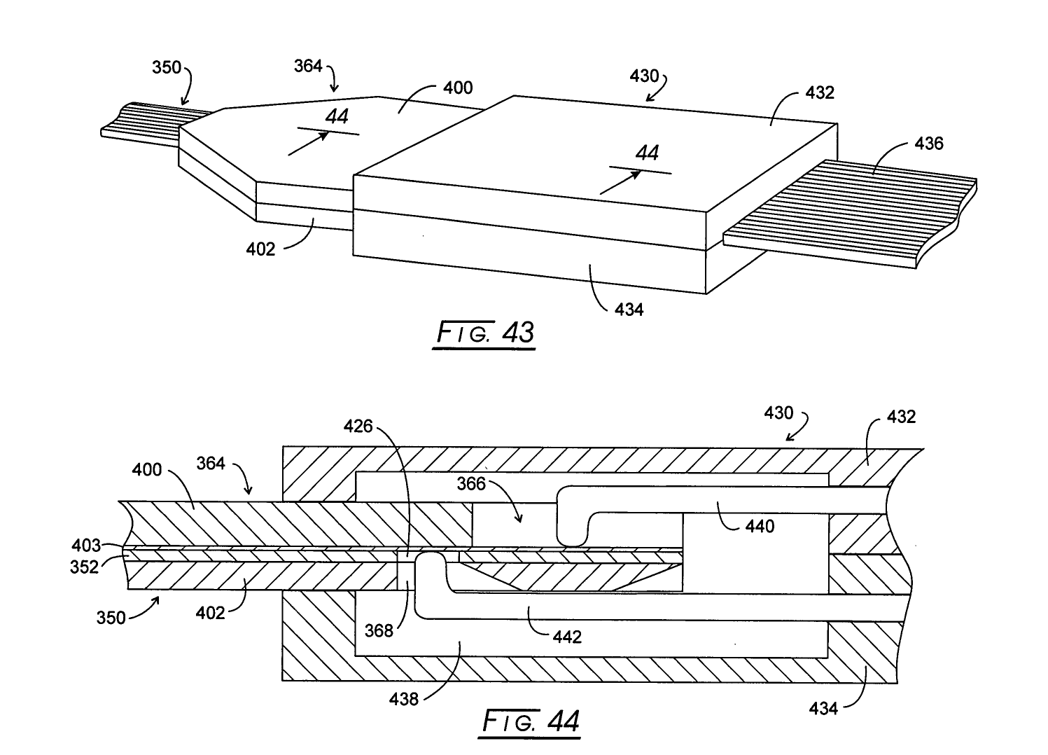

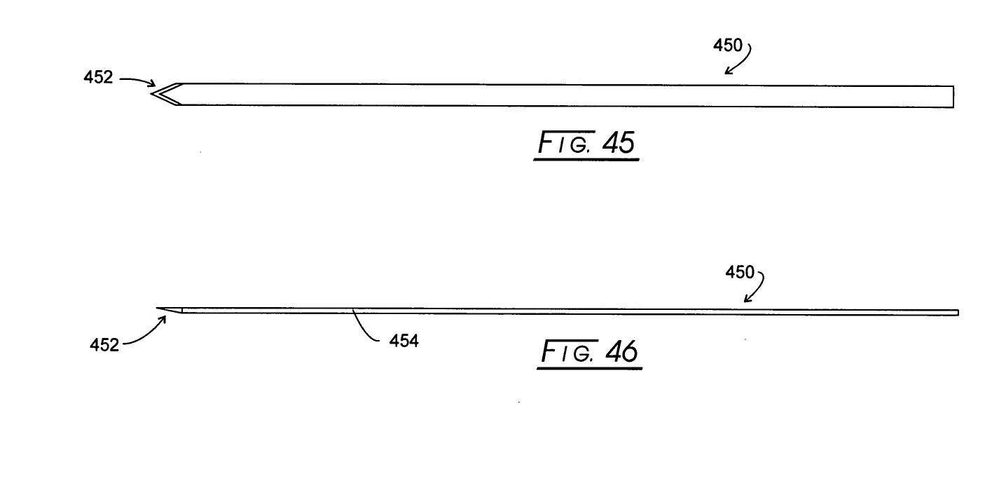

Referring to FIG. 43, connector guide 364 reappears with its assembled components 400 and 402 in conjunction with a cable connector represented generally at 430. Connector 430 is formed of two polymeric components 432 and 434 which are seen to engage a ribbon-type multi-lead electrical connector 436. Looking additionally to FIG. 44, connector guide 364 and cable connector 480 reappear. It may be observed that components 432 and 434 define a cavity 438. Within cavity 438 there are located four gold-plated, beryllium-copper cantilever beam contacts, one of which is represented at 440. These four contacts provide electrical connection with electrode lead traces 370-373 (FIG. 38). At the opposite side of flexible circuit 402, seven gold-plated, beryllium-copper cantilever beam contacts engage the seven four-point connection resistor segment lead traces 388-394, one such contact being shown at 442. Note that the forward contacting portion of contact 442 engages the bottom of flexible circuit 403 through window or opening 368 and opening 426 within thermal barrier 352.

The positioning of implants as at 300 and 350 at the interface between dermis and the next subcutaneous tissue layer may involve the preliminary formation of a heating channel utilizing a flat needle introducer or blunt dissector. Looking to FIG. 45, such an introducer is represented generally at 450. Device 450 is, for instance, 4 mm wide and is formed of a stainless steel, for example, type 304 having a thickness of about 0.015 inch to about 0.020 inch. Its tip, represented generally at 452 is not “surgically sharp” in consequence of the nature of the noted interface between dermis and fat layer. However, looking to FIG. 46, it may be observed that the tip 452 slants upwardly from bottom surface 454 to evoke a slight mechanical bias toward dermis when the instrument is utilized for the formation of a heating channel.

As discussed in connection with FIGS. 16-18, experimentation determined that where a water-filled conformal container is utilized as a heat sink for the instant procedure, agitation of water near at least the contact surface is desirable. In this regard, it was found that the utilization of a conventional laboratory magnetic stirring assembly was quite effective. Measurement of the effectiveness can be carried out by immersing tea leaves or some similar flocculent material within the container to observe the degree of liquid agitation.

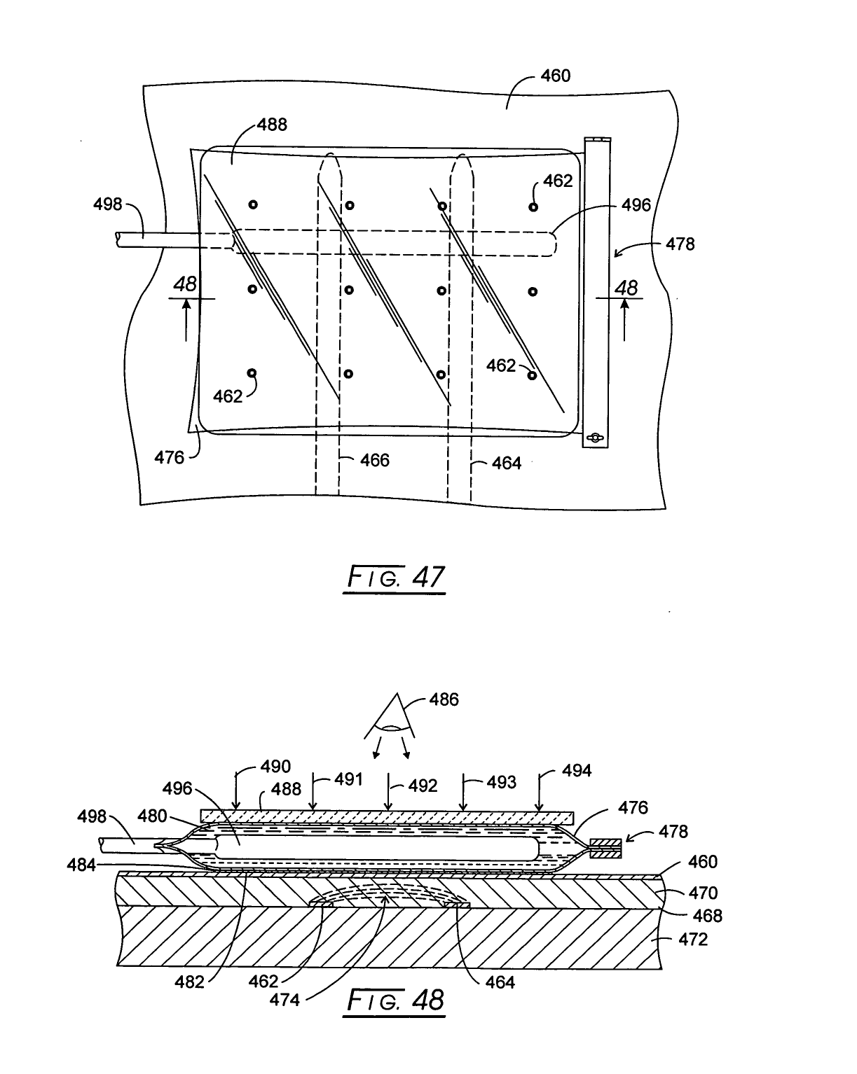

Another approach is represented in FIGS. 47-49. In FIG. 47, epidermis is represented at 460 having a matrix of indicia located on the surface thereof, certain of which are represented at 462 showing digitally recorded initial positions as white centers and dark circles as the skin carried indicia. Looking additionally to FIG. 48, two single channel implants 464 and 466 have been located in heater channels positioned at the interface 468 between dermis 470 and the next adjacent subcutaneous tissue or fat tissue 472. Dermis heating radiofrequency energy derived current flux is shown in general at 474 extending between implant-mounted bipolar electrodes. A bag-like transparent conformal polymeric container 476 is positioned above the implants 464 and 466 and is seen to be closed or secured by a clamping assembly represented generally 478. FIG. 48 reveals that the container 476 is filled with water as at 480 and its contact surface at 482 is slightly pressed against a water heat transfer and lubricant layer 484. Dermis shrinkage is visualized, for example, from eye station 486 looking through transparent glass plate 488. Pressure applied to the plate 488 is symbolized by force arrows 490-494. For the instant embodiment, water 480 is agitated by the inflation and deflation of an elongate bladder 496 having a pneumatic input/output pipe 498.

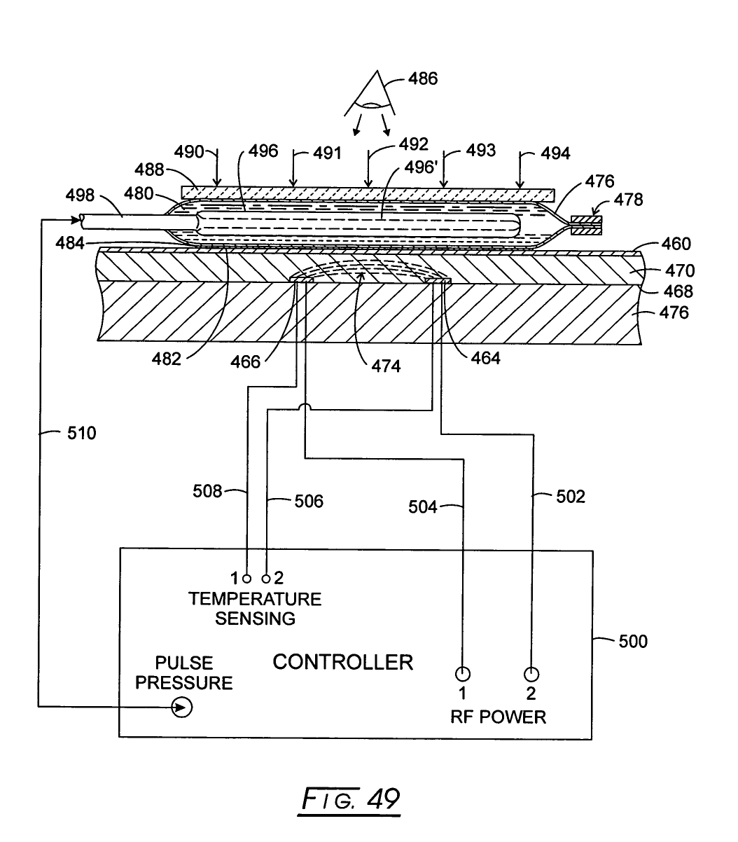

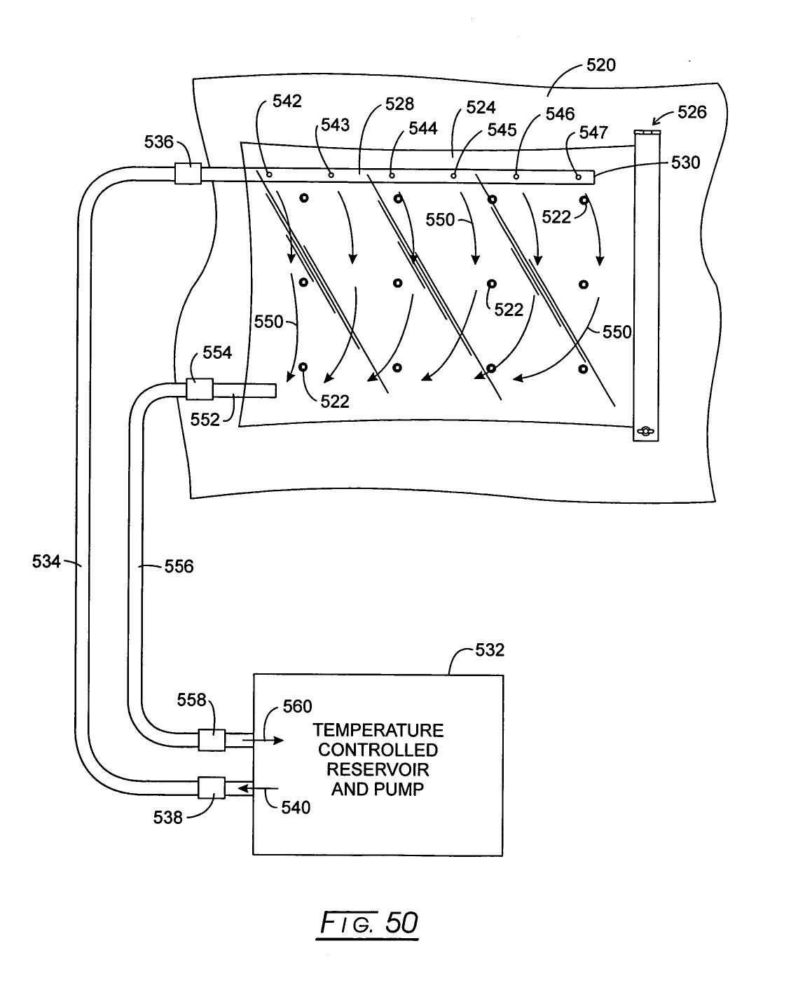

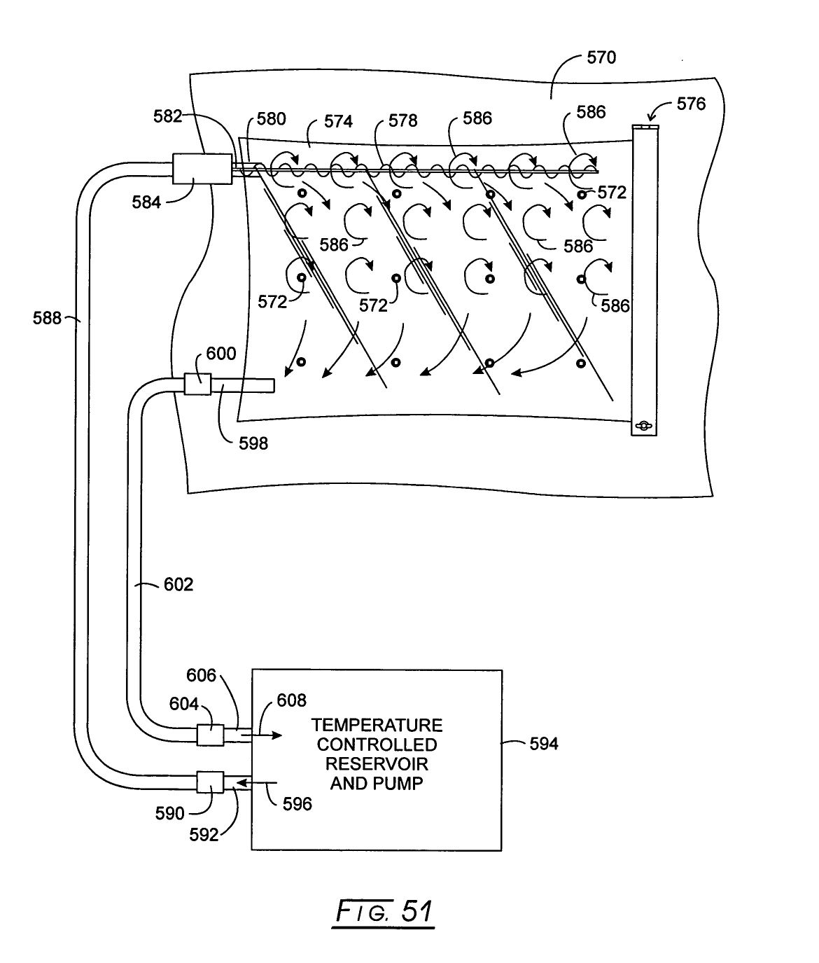

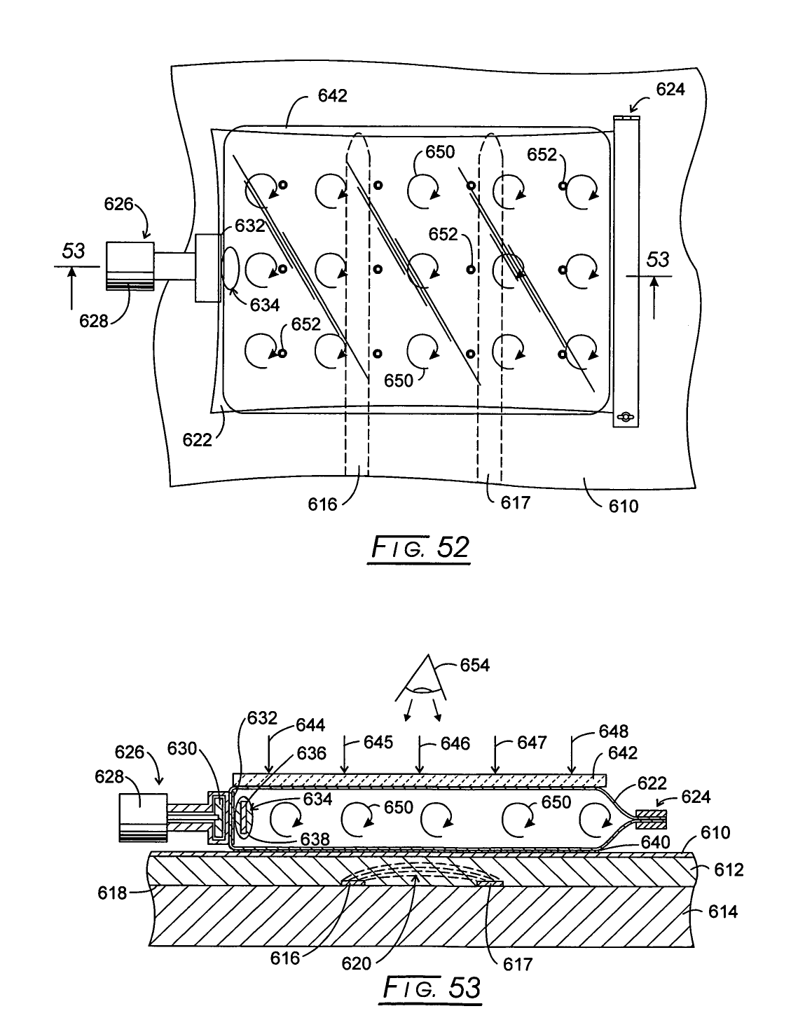

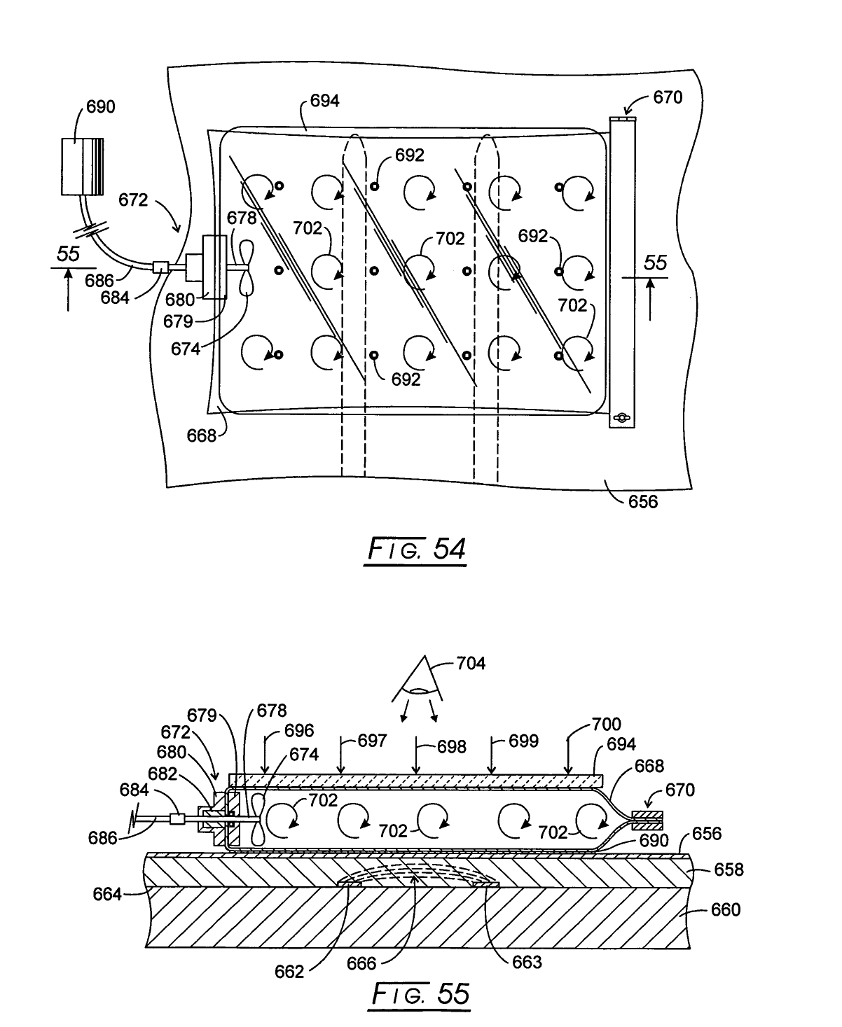

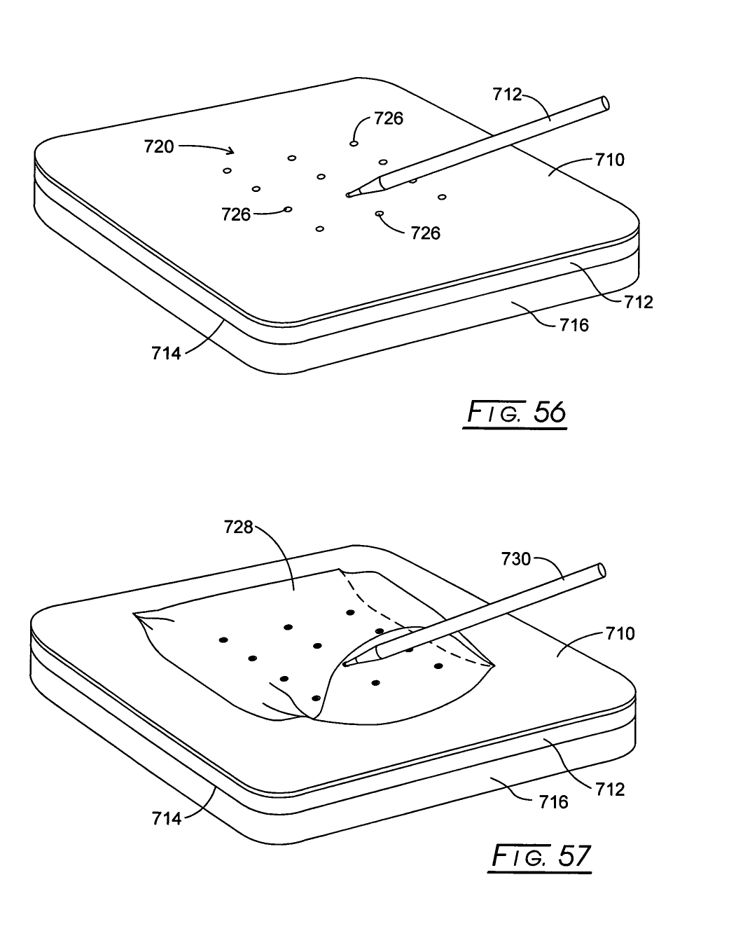

Looking to FIG. 49, FIG. 48 is reproduced in conjunction with a schematically portrayed controller 500. Controller 500 is illustrated providing bipolar radiofrequency power to the electrodes of implants 464 and 466 as represented by respective lines 502 and 504. The temperatures of those electrodes are monitored by corresponding resistor segments and the coupling of controller 500 with the resistor segments at implants 464 and 466 is represented by respective lines 506 and 508. Elongate bladder 496 is shown in its deflated orientation at 496′. Pipe 498 is shown coupled with a pneumatic pulse pressure output by dual arrow 510 extending between the controller 500 and pneumatic pipe 498. In this regard, an oscillating pressure source of air may be provided having a frequency from about 0.5 to 2 cycles per second. It may be observed that for these liquid filled devices, the water within them may be preheated to a desired starting setpoint temperature.

Referring to FIG. 50, a conformal heat sink arrangement is depicted wherein temperature controlled water is circulated within a polymeric container. In the interest of clarity, implants, a glass plate and the like are not shown. However, the surface of epidermis is shown at 520 over which a matrix of visible indicia have been located and the initial positions thereof digitally recorded. Certain of these indicia are represented at 522. As before, the central white square portions of these indicia are the digitally memorized components and the circular indicia are those placed upon surface 520. The polymeric bag or conformal container of the heat sink function is represented at 524 which will be resting against surface 520 with an intermediate thermal transfer and lubricating layer of water therebetween. The bag-type container 524 is closed by a clamping assembly represented generally at 526. Shown extending within and across the length of container 524 is a multi-orifice water distribution pipe or conduit 528 which may be both transparent and flexible. The conduit 528 is plugged at its distal end 530 and it is supplied a flow of temperature controlled water from a reservoir and pump 532 via a flexible polymeric conduit 534. In this regard, conduit 534 is coupled to conduit 528 at a connector 536 and to the reservoir and pump at a connector 538. The outflow of water is represented at arrow 540. Depending upon the apparatus and conduit lengths involved, the conduit 534 may be provided with a thermally insulative covering. The setpoint heat sinking temperature for the fluid involved is controlled at reservoir and pump 532 and the setpoint temperature for such devices will be in the range of about 15° C. to 25° C. Fluid is circulated from orifices 542-547 of conduit 528 as represented by the flow arrows certain of which are shown at 550, to return to reservoir and pump 532 through a relatively shorter outlet conduit 552. Conduit 552 is coupled by a connector 554 to a flexible return conduit 556 which, in turn, communicates with the pump and reservoir 532 from connector 558. Fluid return to the pump and reservoir 532 is represented at arrow 560.

Re-circulating heat sink assemblies as described in FIG. 50 also can be implemented with a mechanical form of water agitation and circulation. Looking to FIG. 51, a mechanically implemented water circulating approach is illustrated. In the figure, a surface of epidermis is shown at 570 upon which a matrix of dot indicia is positioned. Certain of those dot indicia are represented by the common numeration 572. As before, the dot indicia are shown surmounting a white square representing the initial dot position before the procedure occurred which is recorded in digital memory. A transparent conformal container or bag is represented at 574 positioned over the epidermis surface 570. Container 574 is closed with a clamping assembly represented generally at 576. Within container 574 there is located a rotatably mounted polymeric screw mechanism represented generally at 578. Screw mechanism 578 is supported for a rotation at a water input tube 580 and is seen to have a centrally disposed shaft 582 rotatably extending from a fluid drive component 584 which delivers water under pressure into the tube 580 to effect rotation along with rotational agitation of water within container 574 as represented by the generally “C-shaped” arrows, certain of which are identified at 586 representing the generation of water eddy currents. Temperature controlled water input to drive 584 is from flexible conduit 588 which extends to fluid coupling 590, connected, in turn, to the outlet conduit 592 of a temperature controlled reservoir and pump 594. The controlled temperature water output is represented at arrow 596.

Water within the container 574 is returned to the reservoir and pump 594 through an output conduit or pipe 598 extending to a fluid connector 600. Flexible fluid return conduit 602 extends from connector 600 to fluid connector 604. Connector 604, in turn, is coupled with an input pipe or conduit 606 communicating with the temperature controlled reservoir and pump 594 as represented by arrow 608.

As described earlier herein, certain experimentation was carried out utilizing a conventional laboratory stirrer as a heat sink water agitator. Looking to FIGS. 52 and 53, such an arrangement is depicted. In the figures, epidermis is represented schematically at 610; dermis at 612; and next adjacent subcutaneous tissue or fat layer at 614. Two RF electrode-based implants 616 and 617 are located at the interface 618 between dermis 612 and next adjacent subcutaneous tissue or fat layer 614. RF current flux between bipolar electrodes (not shown) is represented generally at 620. Positioned over the outer surface of epidermis 610 is a transparent conformal container or bag 622 which encloses water and is secured by a clamp assembly represented generally at 624. At the opposite side of the container 622 there is located a magnetic stirring assembly represented generally at 626. Assembly 626 includes an electric motor 628, the output shaft of which drives a magnet 630 (FIG. 53). Opposite magnet 630 and within the container 626 is a polymeric flat plate 632 and freely immersed within container 622 adjacent plate 632 is an ellipsoidal magnet stirring rod represented generally at 634 and seen in FIG. 53 as being comprised of a rod magnet 636 embedded within a polymeric capsule 638. That figure also reveals a layer of water 640 functioning as a thermal transfer and lubricating medium. Slight pressure is asserted through the container 622 from a transparent glass plate 642 as represented by force arrows 644-648. Water agitation is represented by curled arrows certain of which are identified in the figures at 650. FIG. 52 reveals a matrix of visible indicia or dots representing an initial condition. Certain of these indicia are identified at 652 as black dots, the centers of skin which are represented as a small white square corresponding with an initial digital recordation of the indicia prior to commencement of therapy. These dots may be viewed by the clinician as represented in FIG. 53 at eye station 654.

Direct agitation of the water within the conformal container heat sinks also can be developed utilizing a conventional impeller. Looking to FIGS. 54 and 55, such an arrangement is schematically depicted. In FIG. 55, epidermis is shown at 656; dermis at 658; and next adjacent subcutaneous tissue or fat layer 660. Adjacent parallel implants 662 and 663 are located in heating channels at the interface 664 between dermis 658 and fat layer at 660. Radiofrequency-based current flux is represented generally at 666 extending between the bipolar electrodes (not shown) of implants 662 and 663. Positioned over the epidermis 656 is a transparent conformal container or bag represented generally at 668 which retains water and is closed at a clamping assembly represented generally at 670. Immersed in the water within container 668 is a driven propeller assembly represented generally at 672. Assembly 672 includes a propeller blade 674 mounted for driven rotation on a shaft 678 extending through polymeric seal plates 679 and 680. Plates 679 and 680 are retained against each other by machine screws (not shown) and the shaft 678 is seen in FIG. 55 to extend through a water seal bushing 682. The shaft also is connected at a connector 684 with a flexible drive shaft 686 extending in driven relationship to an electric motor seen in FIG. 54 at 690. FIG. 55 further reveals a layer of water 690 which functions to provide enhanced thermal exchange and lubrication between the heat sink and epidermis 656. FIG. 54 also is seen to illustrate schematically a matrix of visible indicia, certain of which are identified at 692. Indicia 692 comprise black dots located at the epidermis 656 and interiorly disposed white squares representing the initial position of the dots as digitally recorded. Pressure is applied to the container 668 by a sheet of transparent glass 694 as is represented by the force arrows 696-700 while water agitation is represented by curled arrows, certain of which are identified in the figures at 702. In the arrangement shown, the clinician may observe the extent of shrinkage through the transparent glass sheet 694 and transparent conformal container 668 as represented at eye station 704.

In the above discourse, discussion was provided describing a location of a matrix of visible indicia or dots on the surface of epidermis. These indicia may be generated with an alcohol dissolvable ink. Looking to FIG. 56, tissue is schematically portrayed which includes an epidermis 710 underlying which is dermis 712 which establishes an interface 714 with the next adjacent subcutaneous tissue or fat layer 716. An appropriate hand-held ink marker 718 is illustrated forming a matrix of visible indicia represented generally at 720 and fashioned of discrete indicia, certain of which are identified at 726. Where a transparent conformal container or bag is utilized to retain water for heat sink purposes, the inside of the contact surface of the bag may be employed to provide an initial position matrix of the dots or visible indicia prior to the container being filled with water. Looking to FIG. 57, such an unfilled conformal container 728 is seen having been positioned over the matrix 720 (FIG. 56). A clamping assembly has not closed the bag 728 and a marker 730 is shown marking the inside of the contact surface of the container with a matrix of dots or visible indicia which are in registry with those of matrix 720. Subsequent to this marking, the container 728 is filled with water and associated agitation assemblies. Then it is clamped closed and the dots so formed by marker 730 are retained in registry with the indicia as at 726 of matrix 720.

Another approach to developing this visible indicia-based evaluation is illustrated in FIG. 58. In that figure, the surface of epidermis is shown at 738. A template guided and controlled matrix of visible indicia as represented by dots, certain of which are identified at 740 is then marked upon the surface 738. A transparent polymeric conformal container type heat sink or bag as at 742 is provided being filled with liquid and clamped with clamping assembly 744. The contact surface of container 742, i.e., the surface in contact with skin surface 738, however, is formed with a pre-printed grid represented generally at 746, certain intersections of which correspond with the location of dots or indicia 740. With the arrangement, relative motion of the dots or visible indicia 740 can be readily evaluated with respect to grid 746.

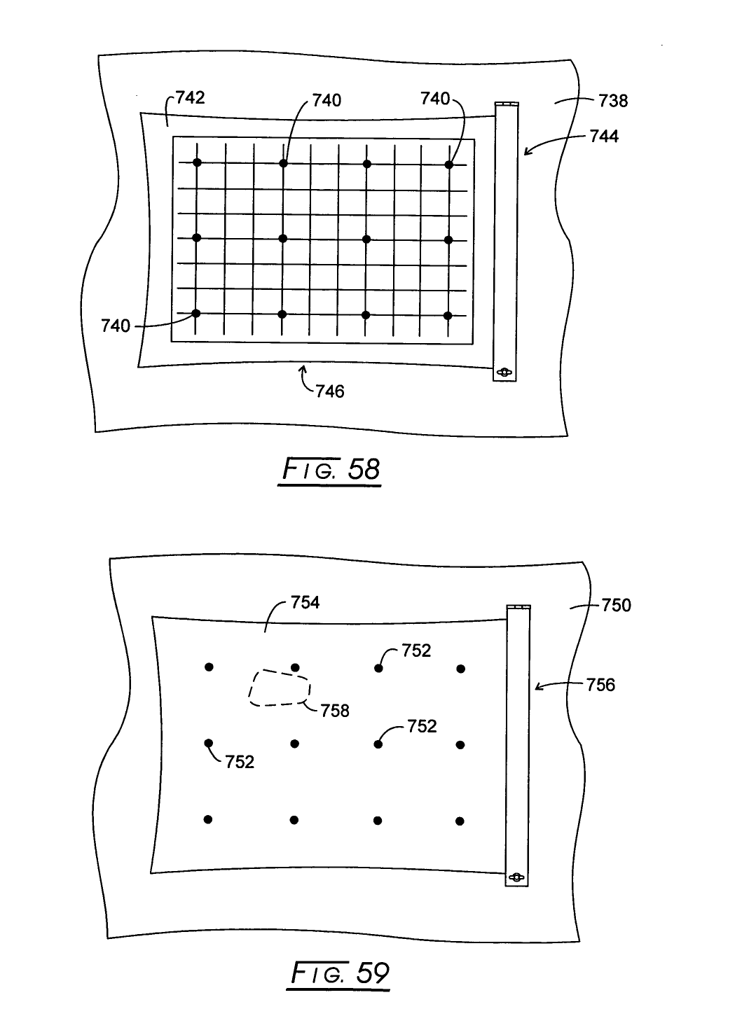

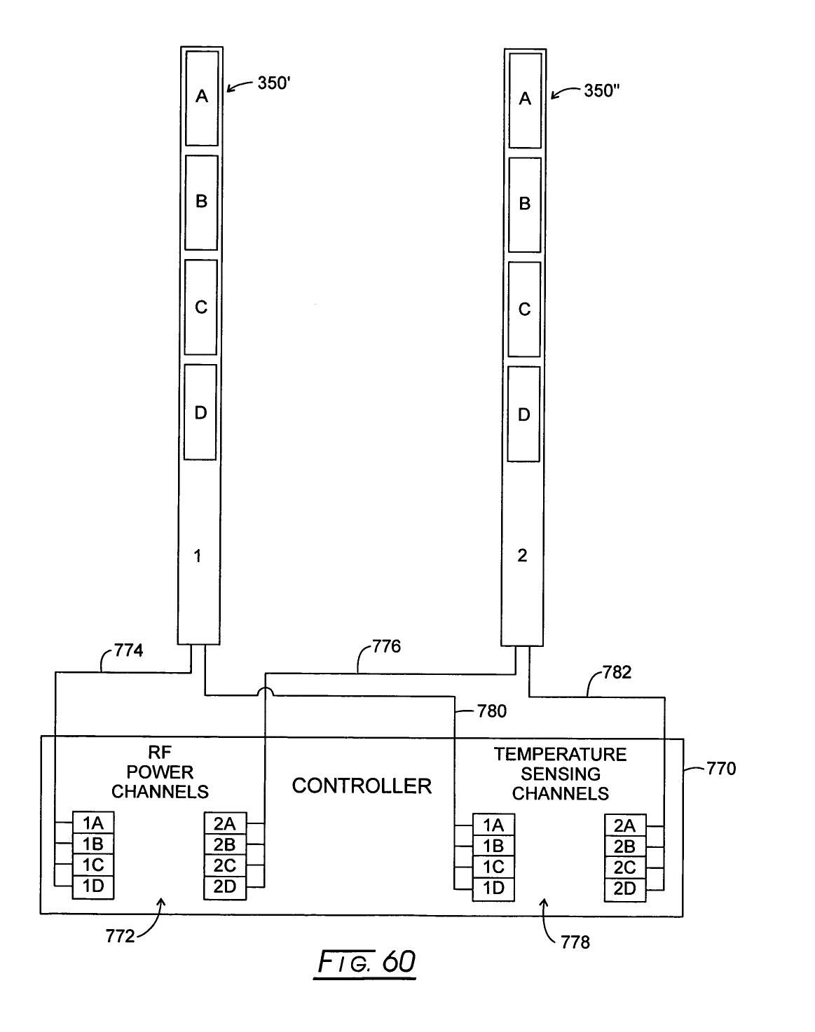

The transparent polymeric conformal containers or bags also can be employed to incorporate a temperature safety indicator. The contact surface of a water-filled heat sink is provided to support a thin transparent layer of reversible thermochromic ink. Should any region of that thermochromic material experience a temperature at or above a skin surface limit temperature, for example, 40° C., then that region will change color and be observable through the transparent heat sink by the clinician. Where such a region is seen, for example, to be changing from clear to a red coloration, the procedure can be shut down immediately. Looking to FIG. 59, the surface of epidermis is represented at 750 again carrying a matrix of dot-like visible indicia, certain of which are represented at 752. Over this matrix region, there is positioned a water-filled transparent conformal heat sink container 754 which is clamped closed by clamp assembly 756 and will incorporate appropriate water agitation and/or circulation assemblies. Shown as a dashed boundary 758 observable through the heat sink as at 754 is a region experiencing a thermochromic color change representing an exceeding of a skin surface limit temperature. The presence of such a region 758 will alert the clinician to terminate the procedure forthwith.

Referring to FIG. 60, a controller arrangement for use with a four electrode implant combined with four resistor temperature sensing segments as described in connection with FIGS. 34-46 is schematically illustrated. Accordingly, in FIG. 60, the four-electrode implants are identified at 350′ and 350″. Note in the figure that the implants are mutually parallel and their electrodes now identified as A-D are laterally aligned so as to assure a radiofrequency current distribution between the laterally aligned paired bipolar electrodes. These electrodes are formed upon a thin polyamide substrate and on the opposite side thereof there is located a rectangular serpentine resistor, the resistance value of which is sampled to determine corresponding electrode temperature. For the instant demonstration, implant 350′ is designated as implant no. 1 and implant 350″ is represented as implant no. 2. A controller for operation in conjunction with implants 350′ and 350″ is schematically represented at 770. Controller 770 performs in conjunction with eight radiofrequency power channels as represented generally at 772. In this regard, one channel of the bipolar system extends to electrodes A-D of implant no. 1 as represented at 1A-1D and line 774. Correspondingly, radiofrequency energy of opposite polarity is provided at electrodes 2A-2D of implant no. 2 as represented by line 776.

The temperature sensing channels of controller 770 are represented generally at 778. In this regard, the resistors located in thermal exchange relationship with electrodes A-D are identified as sensing channels 1A-1D which function to monitor implant no. 1 as represented at line 780. Correspondingly, temperature sensing resistor channels are identified as 2A-2D with respect to implant no. 2, monitoring being represented at line 782. With this control arrangement, radiofrequency power may be applied in any of a variety of scenarios. For instance, when a setpoint or target temperature has been reached the level of power may be reduced by a given percentage as discussed in connection with FIG. 23. As discussed in connection with FIGS. 11-13, the shrinkage of collagen under the influence of radiofrequency current may be quite rapid and the spacing between the implants, for instance, 15 mm center-to-center may be large enough to develop the highest heat generation between implants and their associated thermal barriers. That effect has been discussed in connection with FIG. 19. Accordingly, it is important that the amount of heat conduction to the next adjacent subcutaneous tissue layer or fatty layer be controlled to avoid any damage to that layer. Such control may include precautions as described in connection with FIG. 22, for example, pre-cooling that layer and providing post therapy heat sink application.

A therapy involving multiple electrode implants as at 350 typically will encompass a skin region wherein four mutually parallel implants will be employed. As before, the corresponding bipolar associated electrodes are aligned in lateral adjacency. Looking to FIG. 61, such an arrangement is illustrated in conjunction with implants A-D. The mutual spacing between adjacent electrodes is designated s₁. Such center-to-center spacing typically will be 15 mm. Additionally, each will exhibit an overall width, w₁₂, of 3 mm. For any grouping of more than three such implants, the outside implants, here implants A and D are arbitrarily designated as outwardly disposed “border” implants. Correspondingly, implants B and C are arbitrarily designated as “inwardly” disposed implants.

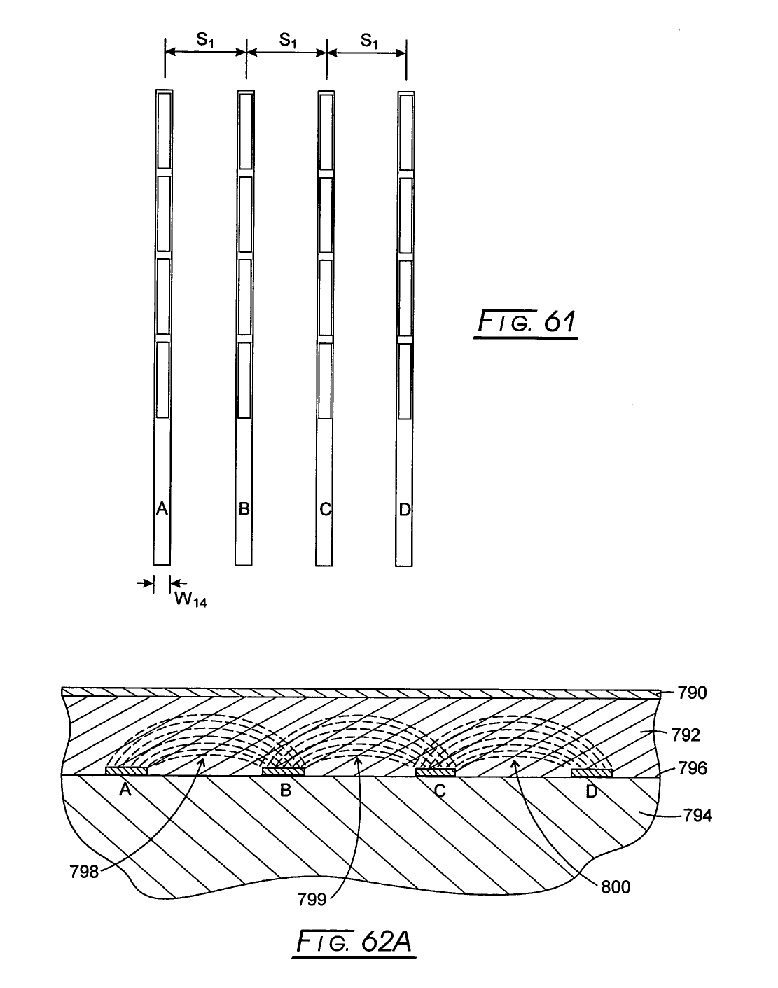

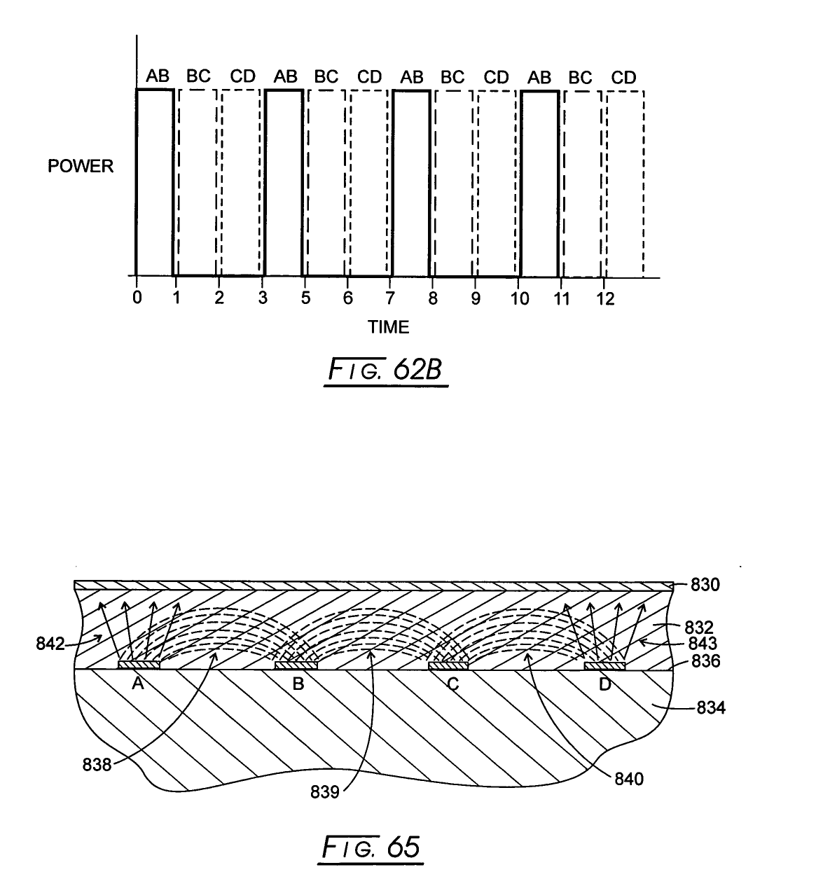

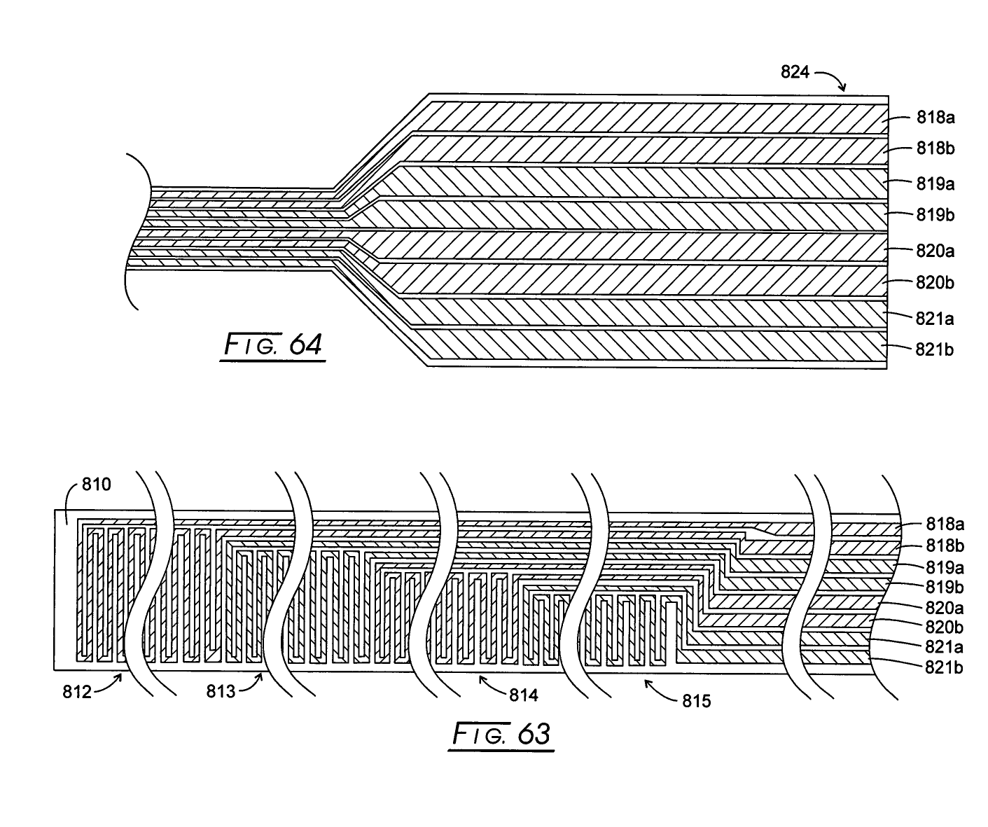

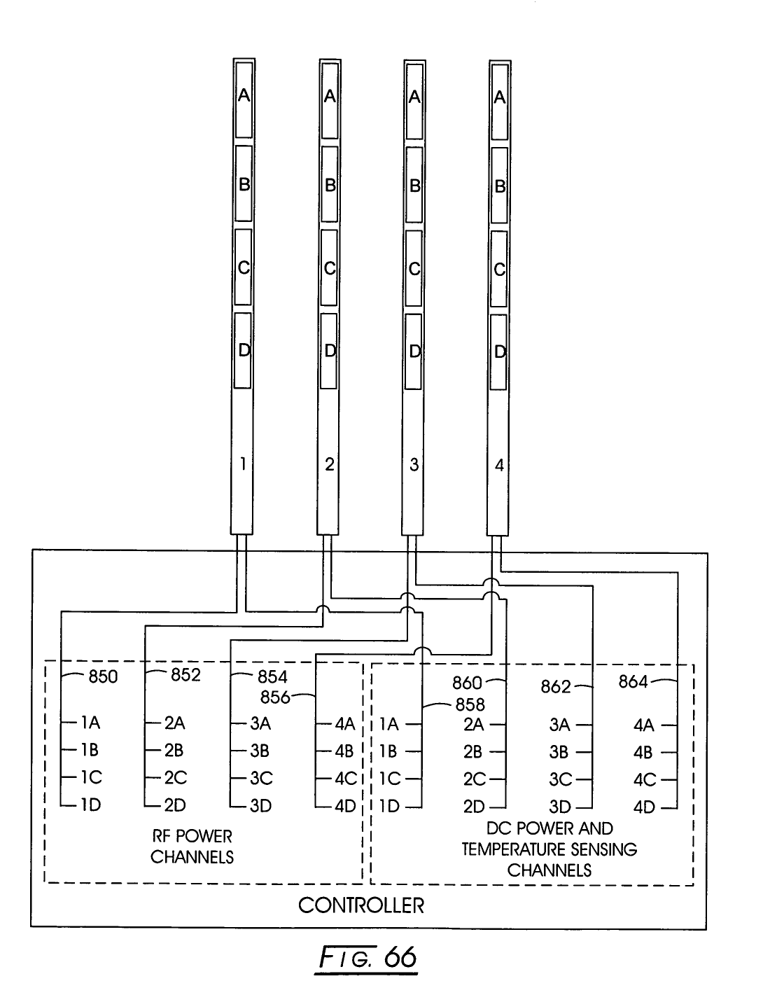

Looking additionally to FIG. 62A, implants A-D are schematically illustrated in section within tissue. In the figure, epidermis is represented at 790; dermis is represented at 792; and the next adjacent subcutaneous tissue or fat layer is represented at 794. Implants A-D are seen to be embedded at the interface 796 between dermis 792 and next adjacent subcutaneous layer 794. Radiofrequency current flux between implants A and D is represented in general at 798. Such current flux between implants B and C is represented generally at 799; and radiofrequency current flux between implants C and D is represented in general at 800. Electrodes A-D are energized in paired bipolar fashion, for instance, employing a 50% duty cycle. Looking additionally to FIG. 62B, an energization versus time diagram is revealed. In the figure, border implant A and next adjacent inwardly disposed implant B are energized as represented at AB. Next, inwardly disposed implants B and C are energized in bipolar fashion, following which inwardly disposed electrode C and the border electrode D are energized in bipolar fashion. The sequence then continues to repeat itself and the time interval for each bipolar energization will be from about 10 to about 11 milliseconds. Observation of the diagram of FIG. 63B reveals that the border implants appear to receive one half of the amount of radiofrequency energy as the inwardly disposed implants. In general, this may develop an inherent “feathering” at the border of the region of skin being treated. Where additional heat energy is desired at the location of the border implant, then a hybrid implant, inter alia, may be employed at those border locations. In this regard, instead of the resistor segment associated with each electrode being a temperature sensor, the resistors are configured and connected to be both resistive heaters and temperature sensors. With this arrangement, the implant will closely resemble that described at 350 but the lead structure extending to the resistor segment changes for direct current heating drive as well as intermittent temperature sensing. Additionally, the thickness of the resistor trace and lead trace components may be at the thicker end of the earlier-described range, i.e., to a value of about 0.005 inch. Looking to FIG. 63, the inward surface of a polyamide (“Kapton”) substrate is represented at 810. Supported upon the substrate 810 inward surface are four resistor heater and temperature sensing segments represented generally at 812-815. Leading to these heater segments 812-815 are paired lead traces shown respectively at 818 a, b-821a, b.

Turning to FIG. 64, the trailing end of the hybrid implant is represented generally at 824 to which these leads extend and are identified with the same alphanumeric identification. A later investigation of the resistor implemented heater/temperature sensor implant structuring showed that its function can be operationally improved through utilization of the earlier-described 4-point lead topology. With such an arrangement, an accommodation for the impedances associated with cabling, leads and the like is not required. Returning momentarily to FIGS. 39 and 40, such a 4-point topology has been described. To operate such a resistor-based implant structure for heating purposes, heat energy is applied through the sensing leads as shown at 389-393, while the low level d.c. source leads 388 and 394 are used only for deriving temperature responsive resistance values. For this purpose, resistance is intermittently sampled, for example, a 10-100 microsecond interval following which a power cycle ensues for 100 milliseconds. By so generating heat at hybrid implants, for example located as “border” implants, FIG. 62A becomes changed as shown in FIG. 65. Looking to the latter figure, a schematic representation of epidermis 830; dermis 832; next adjacent subcutaneous tissue or fat layer 834 again appears in schematic fashion. Implants A-D again are identified as being located adjacent the interface 836 between dermis 832 and fat layer 834. As before, bipolar radiofrequency derived current flux between implants A and B is represented generally at 838; current flux between implants B and C is identified generally at 839; and current flux between implants C and D is identified generally at 840. However, note that resistive heat is portrayed as issuing from border implant A as represented generally at arrows 842 while the same form of heat is additionally generated from border implant D as represented generally at 843. The general structure of the controller additionally changes from that described in connection with FIG. 60. In this regard, a controller incorporating features for additionally operating hybrid implants is shown in FIG. 66. Looking to that figure, implants 1, 2, 3 and 4 are identified in a manner similar to FIG. 60. However, implants 1 and 4 are hybrid implants and are seen to be located as “border’ implants. In the schematic, radiofrequency power channels 1A-1D are represented in energizing relationship with the electrodes of border implant 1 as represented at line 850. Radiofrequency power channels 2A-2D are represented as providing radiofrequency based power to the electrodes of implant 2 by line 852. Radiofrequency power channels 3A-3D are shown in energizing association with the electrodes of implant 3 by line 854; and radiofrequency power channels 4A-4D are shown to be in energizing relationship with the electrodes of implant 4 by line 856. Four-point temperature sensing channels 2A-2D are seen to be associated with the resistor segments of implant 2 by line 858; and four point temperature sensing channels 3A-3D are seen to be operatively associated with the resistor segments of implant 3 by line 860. For hybrid implant 1 the resistive heating and temperature sensing channels associated with the resistor segments thereof are shown at 1A-1D and the association of those channels with the resistor segments of implant 1 is represented at line 862. In similar fashion, resistive heating and temperature sensing channels 4A-4D are seen to be associated with the resistive segments of implant 4 by line 864.

Now considering the use of resistor segments to measure temperature at the situs of the RF electrodes, once the implant has been located within heater channels and preferably following the positioning of a heat sink at the skin region of interest, the temperature of the segment prior to therapy for the energizing of either the RF electrodes or heater resistors if such heaters are utilized in the hybrid form of implant is determined. For example, this predetermined resistor segment temperature, T_{RS,t0}, based on an algorithm related to the measured skin surface temperature, T_{skin,t0}, which may be expressed as follows:

T_{RS,t0}=f(T_{skin,t0}). (2)

As an example, this computed temperature may be 35° C. Also predetermined is the treatment target or the setpoint temperature. That temperature may be based upon radiofrequency heating at constant power as described in connection with FIGS. 9 and 10; a setpoint temperature at which the power level applied will be diminished as described in connection with FIG. 23; or a combination of temperature ramping up and a subsequent diminution of power applied at constant power applied at described later herein.

When the controller is instructed to commence auto-calibration the following procedure may be carried out:
a. The controller measures the resistance of each resistor segment preferably employing a low-current DC resistance measurement to prevent current induced heating of those resistors.
b. Since the resistor component is metal having a well-known, consistent and large temperature coefficient of resistance, α having a value preferably greater than 3000 ppm/° C. (a preferred value is 3800 ppm/° C.), then the target resistance for each Resistor Segment can be calculated using the relationship:

R_{RSi,target}=R_{RSi,t0}(1+α*(T_{RS,t}−Tₜₒ)) (3)
where:
R_{RSi,t0}=measured resistance of Resistor Segment, i, at imputed temperature of Resistor Segment under skin, T_{RS,to}
α=temperature coefficient of resistance of resistor segment.
T_{RS,t}=target or setpoint treatment temperature.
T_{RS,t0}=Imputed temperature of RF electrodes for the combined temperature of resistor heater/sensor and RF electrodes residing under the skin and prior to the start of any heating of them.

For four-point sensor resistor connections, no accommodation need be made for the impedance exhibited by the cable extending to the controller. On the other hand, for any hybrid based implants without the 4-point approach accommodation must be made in the control algorithm for that impedance characteristic. Temperature evaluations are made intermittently, for example, every 500 milliseconds and the sampling interval may be quite short, for example, 2 milliseconds.

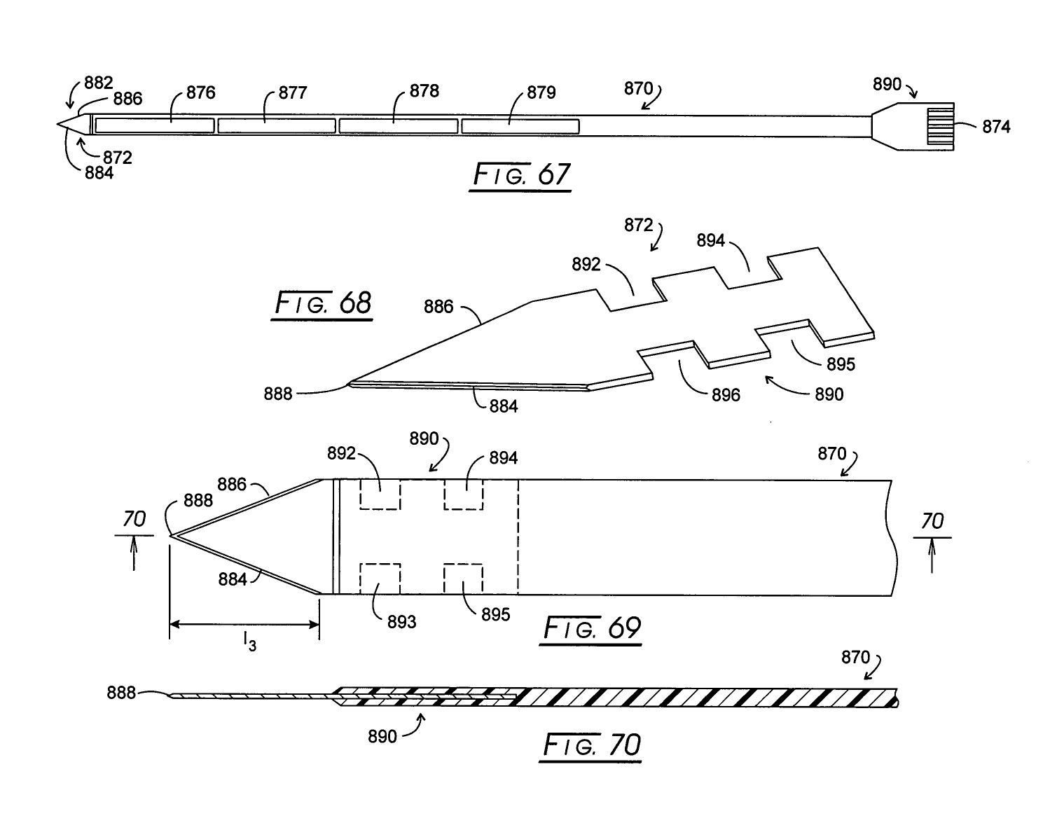

A stainless steel flat dissecting instrument 450 has been described in connection with FIGS. 45 and 46 which has the function of forming a heating channel through an entrance incision prior to locating an implant within the pre-formed channel. However, the thermally insulative generally flat thermal barrier and support component of the implant leading end may be bladed so as to enter a skin entrance incision and guidably move under compressive urging along the interface between dermis and next adjacent subcutaneous tissue to form and be located within a heating channel. The bladed leading end can be established in the course of injection molding of the thermal barrier. Looking to FIG. 67, a bladed implant is represented in general at 870. Implant 870, with the exception of its forward end or tip is configured in the manner described at 350 in connection with FIGS. 34-42. Accordingly, it is formed with an elongate polyetheramide support and thermal barrier extending from a leading end represented generally at 872 to a trailing end shown generally at 874. This thermal barrier supports a polyimide circuit support (Kapton), the outer surface of which carries four gold-plated copper radiofrequency energizable electrodes as seen at 876-879. A connector guide represented generally at 880 is located adjacent trailing end 874. Leading end 872 of the thermal barrier now supports an introducer tip identified generally at 882. Tip 882 will permit the clinician to insert the implant 870 at the interface between dermis and next adjacent subcutaneous tissue or fat layer with the optional use of a separate introducer dissecting device.

Tip 872 may be formed of a type 304 stainless steel (full hard). Looking to FIG. 68, the tip 872 is revealed in perspective fashion and has a thickness of 0.005 inch and an overall length of 0.380 inch. The tip is configured with two cutting or dissecting edges 884 and 886 extending rearwardly from a point 888 at an included angle of 41°. Rearwardly of the edges 884 and 886 the tip 872 is configured with an embeddable rear portion represented generally at 890. Portion 890 is seen to be configured with embedding notches 892-895. Looking additionally to FIG. 70, a sectional view reveals embeddable rear portion 890 as it is located within the thermal barrier as a consequence of an injection molding process. The blade edges 884 and 886 extend axially to point 888 a distance, l3 which is 0.160 inch.

In use, the clinician forms a small incision within the skin at the heating channel entrance location then manually inserts the bladed implant 870 through that incision in a manner wherein it will bluntly dissect and be located within a heating channel positioned at the interface between dermis and the next adjacent subcutaneous tissue or fat layer.

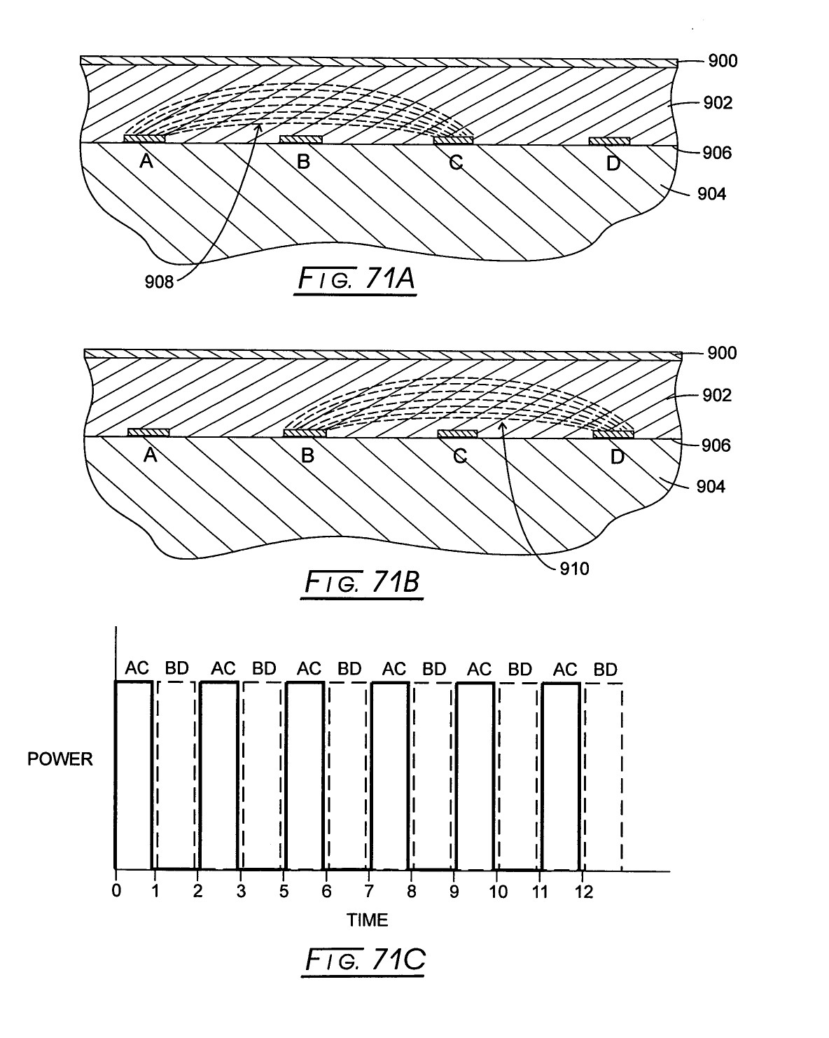

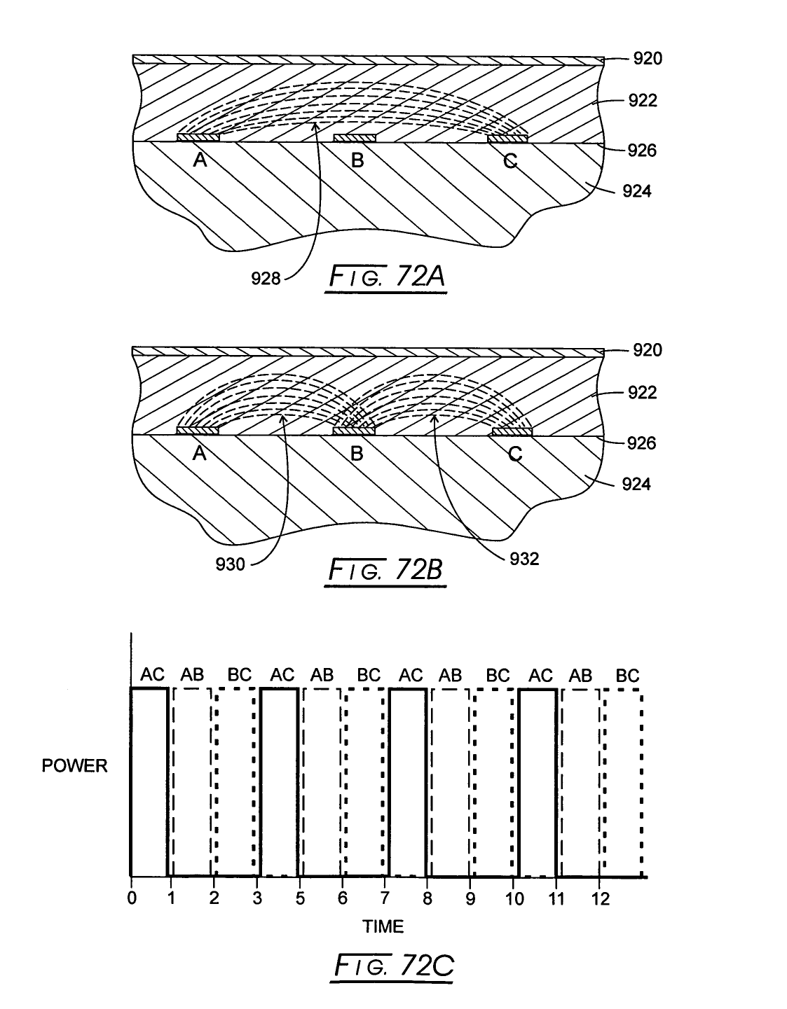

FIGS. 62A and 62B have illustrated a sequence for bipolar radiofrequency excitation of the electrodes of four spaced-apart parallel implants wherein working under a duty cycle approach, successive implants in a sequence of four were excited. The sequence described in connection with these figures is one wherein the outer or “border” implants appear to receive half the amount of energy as the “inner” two implants. With such an arrangement an inherent “feathering” can be accomplished within the skin region under therapy. As described in connection with FIG. 65, the border implants may be implemented as hybrid implants combining resistive heating with radiofrequency-based bipolar heating. In FIGS. 71A-71C a sequencing and duty cycle approach is illustrated which provides for equal energy delivery to all implant electrodes without utilization of a hybrid device.

Looking to FIG. 71A, epidermis is schematically represented at 900; dermis at 902 and next adjacent subcutaneous tissue or fat layer at 904. Radiofrequency energized implants A-D are shown located at the interface 906. In the figure, as represented by the radiofrequency current flux path shown generally at 908 the electrodes of implants A and C are excited in an alternating fashion wherein implant B is not excited. Looking to FIG. 71B, the electrodes of implants B and D are excited with bipolar radiofrequency energy as represented by the current flux path shown generally at 910. FIG. 71C illustrates schematically the sequence at hand and it may be observed from that figure that each implant appears to receive the same amount of radiofrequency energy including both the interior and border implants.

It may be recalled in connection with the discussion of the experiment performed in conjunction with the heat sink of FIGS. 19-21 that dermis so heated exhibits a highest temperature halfway between two bipolar excited electrodes. Note in FIG. 71A that implant B now being a passive implant with a thermal shield or barrier is located under what will become that hottest part of the dermis to function to protect subcutaneous fat layer 904 from conductive heating. This same phenomena occurred where alternating implants B and D are excited and implant C is now a passive thermal barrier located halfway between implants B and D.

Equalized radiofrequency-based energy also can be envisioned where three parallel spaced-apart implants are employed. Looking to FIGS. 72A and 72B, epidermis is schematically represented at 920; dermis at 922; and the next adjacent subcutaneous tissue or fat layer 924. Three radiofrequency energized implants labeled A-C are positioned at the interface 926 between layers 922 and 924 represented in the sequencing and timing diagram at FIG. 72C, FIG. 72A shows an initial alternating sequencing step wherein bipolar radiofrequency energization is developed as represented by current flux path lines 928 between implants A and C. This locates a passive implant B having a thermal barrier halfway between implants A and C and thus at the hottest portion of heated dermis to ameliorate thermal conduction into the fat layer 924.

FIG. 72B and FIG. 72C shows the next two steps in the duty cycle based sequencing. The next step in the sequence provides bipolar excitation with respect to implants A and B as represented at current flux path lines 930. The third step in the repeating sequence provides for the bipolar radiofrequency energization of implants B and C as represented by current flux path 932. FIG. 72C reveals that this sequence AC, AB, BC then repeats itself during the interval of therapy.

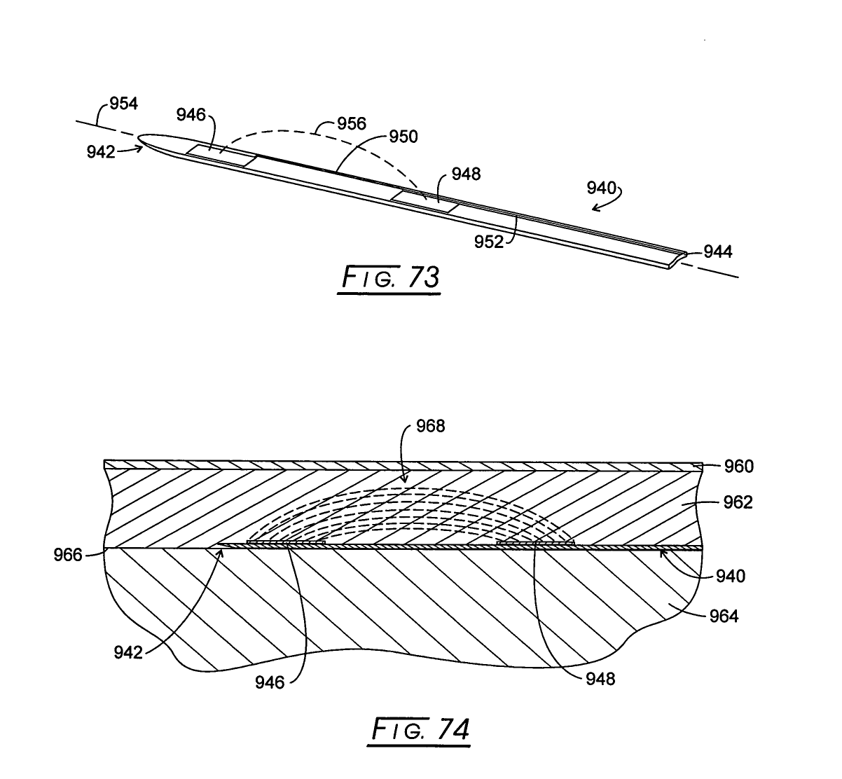

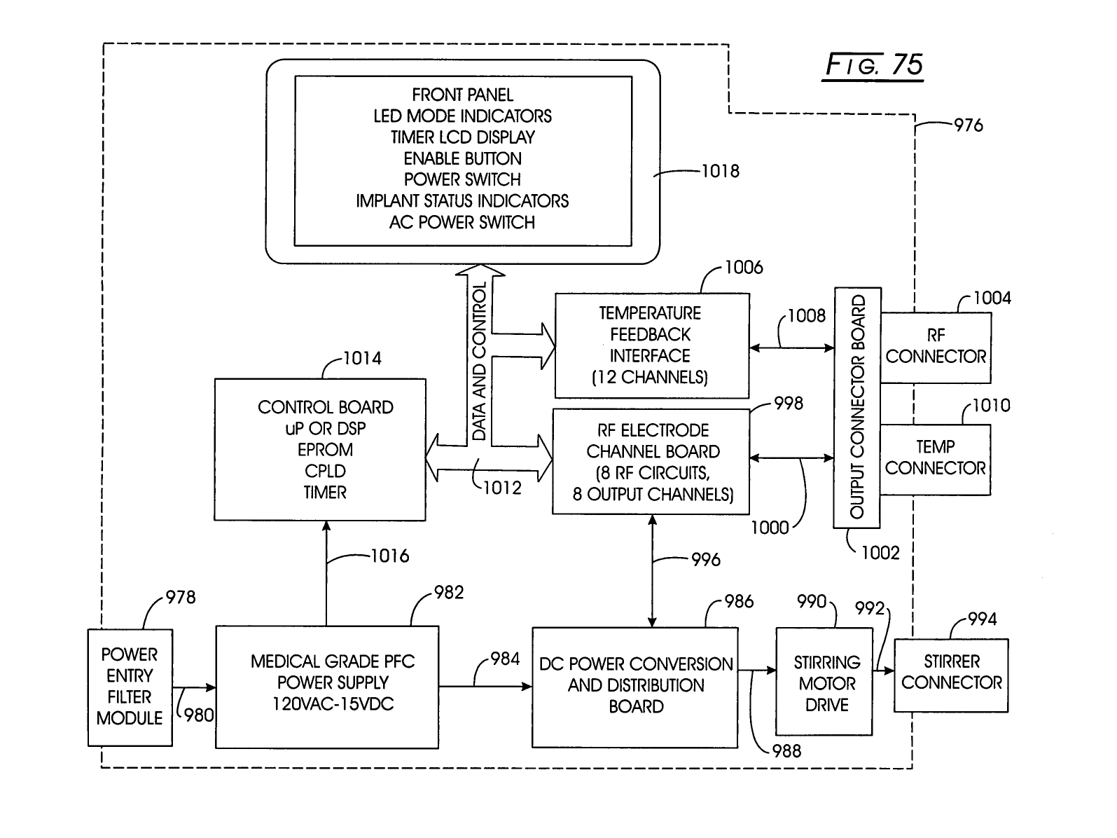

For some applications of the instant technology, only a minor amount of skin region is involved. Under such conditions, the clinician may wish to perform with a single implant carrying spaced-apart bipolar electrodes. Referring to FIG. 73, such an implant is represented in general at 940. With the exception of the size and spacing of the electrodes, implant 940 is configured with dimensions and materials as described in conjunction with implants 300 and 350. In this regard, implant 940 is formed with a polyetheramide support and thermal barrier extending from a forward end represented generally at 942 to a trailing end 944. A flexible circuit (Kapton) having inner and outer surfaces is mounted over the support surface of the thermal barrier. The outer surface of this flexible circuit is seen to support two spaced-apart electrodes 946 and 948. Two corresponding leads as at 950 and 952 extend to the trailing end 944. Gold-plated copper electrodes 946 and 948 preferably will have a length along longitudinal axis 954 of about one half inch and will be spaced apart about one inch. The bipolar association between the electrodes 946 and 948 is represented by dashed curve 956. Located immediately beneath each electrode 946 and 948 and registered therewith is a resistor temperature sensing component (not shown) also having a length along axis 954 corresponding with the length of electrodes 946 and 948. Looking to FIG. 74, schematically represented are epidermis 960; dermis 962 and next adjacent subcutaneous tissue or fat layer 964. Implant 940 is located within a heating channel at the interface 966 between dermis 962 and next adjacent subcutaneous tissue layer 964. The leading end 942 of implant 940 reappears as well as the electrodes 946 and 948. When these electrodes are excited in bipolar fashion with radiofrequency energy, a current flux path represented generally at 968 will function to heat a small zone of dermis 962.

Referring to FIG. 75, a block diagram is presented within dashed boundary 976 representing a control console performing, for example, with three implants, each supporting four RF electrodes and an associated four temperature sensing resistor segments. In the figure, a power entry filter module is represented at block 978 providing a filtered a.c. input as represented at arrow 980 to a medical-grade power supply with power factor correction (PFC) as represented at block 982. By providing PFC correction at this entry level to the control circuitry, the console will enjoy a somewhat universal utilization with various worldwide power systems. The d.c. output from power supply 982 is provided as represented at arrow 984 to a d.c. power conversion and distribution board represented at block 986. As part of the d.c. power distribution, drives can be imparted to a stirring motor as described in FIG. 53 at 628. Such drive is represented at arrow 988 and block 990. Drive 990 is represented at arrow 992 providing rotational input to a stir connector represented at block 994. Block 994, for instance, may be associated with the function of device 630 in FIG. 53. Returning to power conversion and distribution board 986, as represented by dual arrow 996, logic power and radiofrequency energy inputs are provided to a radiofrequency electrode channel board represented at block 998. Channel board 998 will exhibit a topography incorporating eight bipolar radiofrequency circuits and an associated eight output channels. As represented by the interfacing dual arrow 1000 and block 1002, the output channels are directed to an output connector board which is operatively associated with the radiofrequency electrode connector as represented at block 1004. Also associated with the output connector board 1002 is the twelve channel resistor segment temperature feedback interface represented at block 1006 and dual interface functioning arrow 1008. The connector associated with the function of arrow 1008 is represented at block 1010. Control into and from the temperature feedback interface 1006 and the RF electrode channel board 998 is represented at control bus or arrow 1012. The circuit distribution function at 1012 is seen to be functionally associated with a control board represented at block 1014. Such control may be implemented, for instance, with a microprocessor or digital signal processor and will include memory (EPROM). It may also be implemented with a programmable logic array or device (CPLD), and a timing function. Logic d.c. power supply is directed to the control function 1014 as represented at arrow 1016. As represented at circuitry 1012 and symbol 1018 the console 976 incorporates a front panel having user control inputs as well as displays. In this regard, as listed in the symbol, the console employs an a.c. power switch; implant status indicator; a power switch; an enable button or switch; a timer LCD display; and light emitting diode (LED) mode indicators.

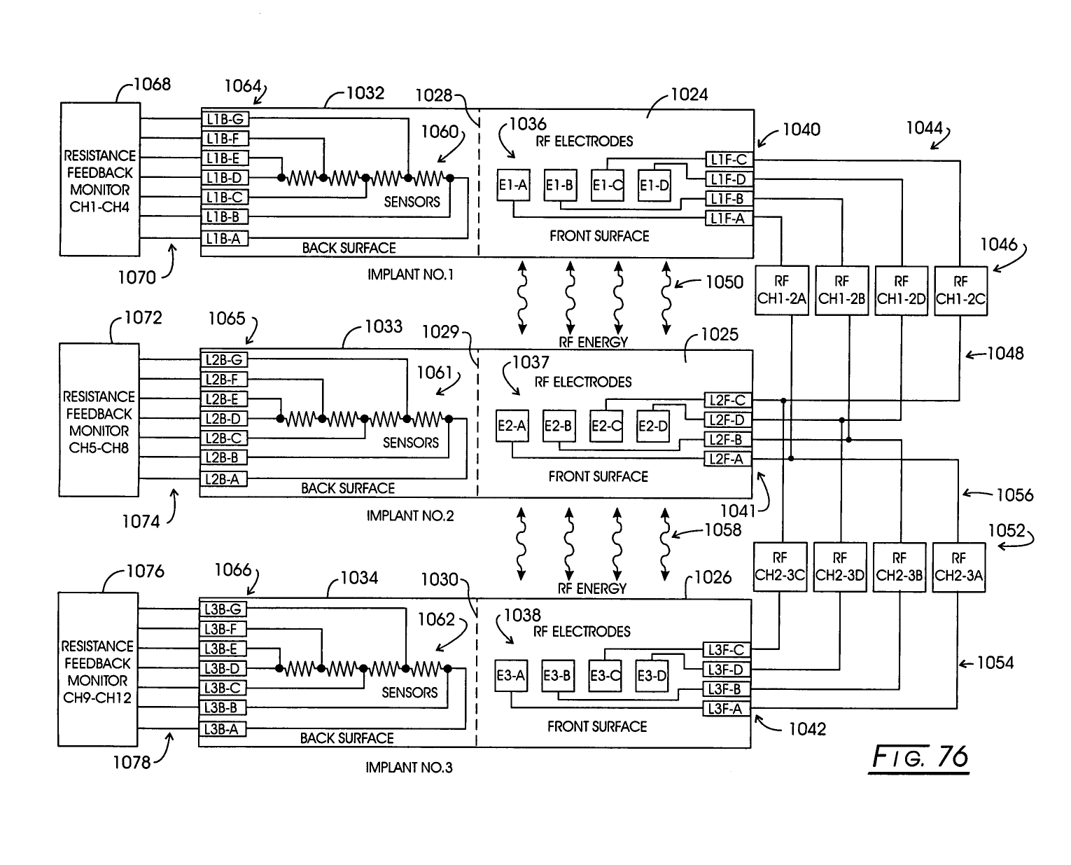

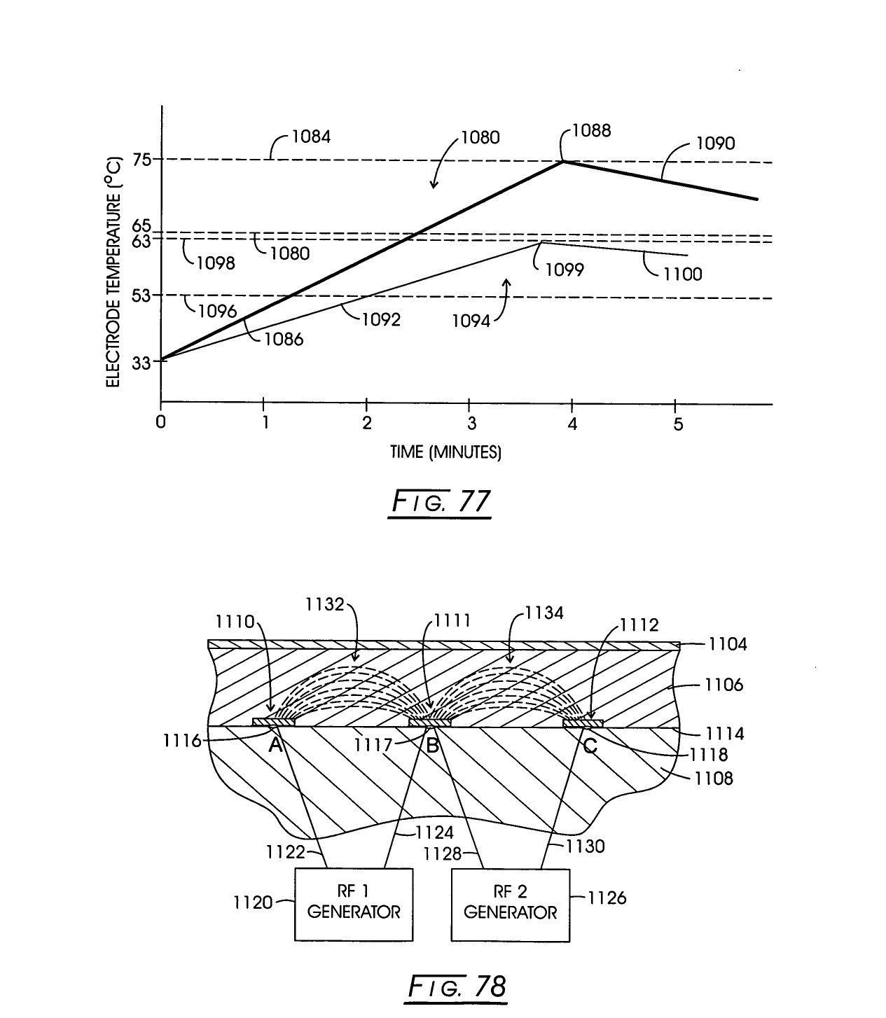

Referring to FIG. 76, schematic representation of the flexible circuit assemblies for three implants numbered 1-3 are presented in combination with the functions of resistance feedback monitoring and bipolar radiofrequency energy channel designations. In the figure, the front or outward surfaces of the flexible circuits of implants 1-3 are represented respectively at 1024-1026. These outward surfaces have been described, for instance, at 358 in FIGS. 37 and 38. Outward surfaces 1024-1026 are delimited from the rearward surfaces symbolically by respective dashed lines 1028-1030. Thus, implants 1-3 are further represented by flexible circuit rearward or back surfaces shown respectively at 1032-1034. Flexible circuit surfaces 1032-1034 correspond with that described at 386 in connection with FIGS. 39 and 40. As described later herein, flexible circuit assemblies also may be fashioned with two discrete substrate layers, one carrying RF energizable electrodes and the other carrying temperature sensing resistor segments. The gold-plated copper electrodes at surface 1024 of implant No. 1 are represented in general at 1036 and are identified as E1-A-E1-D. Correspondingly, upward surface 1025 supports four radiofrequency electrodes represented generally at 1037 which are identified as E2-A-E2-D and outward surface 1026 supports four radiofrequency electrodes represented generally at 1038 and identified as E3-A-E3-D. Electrode arrays 1036-1038 correspond, for example, with the electrodes identified at 360-363 in FIG. 37. Electrodes 1036 are seen to be operationally coupled by leads extending to lead contacts represented generally at 1040 and identified as L1FA-L1FD. Similarly, electrodes of array 1037 are coupled by leads to contact leads represented at 1041 and identified as L2F-A-L2F-D; and the electrodes of array 1038 are coupled by leads extending to contact leads represented generally at 1042 and identified as L2F-A-L2F-D. These contact leads 1040-1042 correspond with the leads represented at 370-373 identified in FIG. 38. Contacts 1040 are seen to be operationally associated by line array 1044 with an array of four output channels represented generally at 1046. These output channels identify the bipolar association between lead contact arrays 1040 and 1041. In this regard, they are identified as CH1-2A-CH1-2D. Such channels have been described in FIG. 75 at block 998. Four channel array 1046 additionally is operationally associated with lead contact array 1041 of implant No. 2 by lead line array represented in general at 1048. For instance, output channel CH1-2A provides a bipolar energization association between contact lead L1F-A of array 1040 and contact lead L2F-A of contact lead array 1041. The bipolar energy association between electrodes E1-A-E1-D and respective electrodes E2-A-E2-D are represented by the energy transfer symbols identified generally at 1050.

In similar fashion, the contact leads of array 1042 of implant No. 3 are operationally associated with a corresponding array of four radiofrequency output channels represented generally at 1052 by line array 1054. In this regard, lead contact L3F-A-L3F-D are operationally associated with respect to output channels CH2-3A-CH2-3D. As represented by the line array identified generally at 1056, the four radiofrequency output channels 1052 are operatively associated in bipolar fashion with the corresponding contact leads 1041 of implant No. 2. In this regard, channels CH2-3A-CH2-3D are associated in bipolar relationship with contact leads L2F-A-L2F-D. This bipolar association provides for electrode-to-electrode energy transfer as represented by the energy transfer symbols identified in general at 1058.

Looking to the inward or back surfaces 1032-1034 of the flexible circuit assemblies of respective implants Nos. 1-3. Three arrays of temperature sensing resistors are identified generally at 1060-1062. Sensing resistor arrays 1060-1062 are coupled by a four-point configured lead array extending to seven lead contacts identified in general respectively at 1064-1066. Resistor arrays as at 1060-1062 have been described in connection with FIG. 39 at resistor segments 380-383, while lead contact arrays as at 1064-1066 have been described in connection with FIG. 40 at 388-394. The four temperature feedback interface channels represented at contact lead array 1064 are represented as being associated with a resistance feedback monitor function for channels 1-4 at block 1068 by the line array represented generally at 1070. In similar fashion, the four channels represented by contact lead array 1065 are operationally associated with resistance feedback monitor channels 5-8 as represented at block 1072 and the line array identified generally at 1074. The four sensing channels represented by four resistor array 1062 and contact lead array 1066 are associated with resistance feedback monitor or channels 9-12 as represented at block 1076 and the line array identified generally at 1078.

The animal studies carried out, for example, as described in conjunction with FIGS. 11-13 and as represented in FIG. 23 led to a determination that temperature elevation of the electrodes and rate of collagen shrinkage was too rapid. Accordingly, an algorithm under which an associated control system was to perform was devised wherein the length of a typical therapy would be expanded to about five minutes. The less desirable shorter therapy intervals were occasioned with the utilization of a constant power output as described above in connection with FIGS. 9 and 10. Studies indicated that the somewhat simple expedient of lowering the power level at constant power was not an acceptable solution. This is because the thickness of the dermis varies. For instance, if the dermis is relatively thick, at lowered constant power thermal transition or threshold collagen shrinking temperature might never be reached, while at higher constant power levels burn damage may be encountered.

Referring to FIG. 77, a plot of desired electrode temperature with respect to therapy time and minutes is presented wherein a controlled ramping-up of electrode temperature into a collagen shrinkage domain over a ramp internal is followed by what is referred to as a “thermal soak” interval. In the figure, a starting temperature is shown to be, for example, 33° C. Above that temperature, for example, between about 65° C. and 75° C. there is established a collagen shrinkage domain represented generally at 1080. Shrinkage domain 1080 is seen to extend between the dashed line level 1082 corresponding with a collagen shrinkage threshold temperature of 65° C. and dashed line level 1084 corresponding with an upper limit level temperature of about 75° C. As represented at electrode temperature versus time curve portion 1086, variable power is applied to the bipolar electrodes as a ramp control commencing at the noted 33° C. and reaching the upper limit of 75° C. within domain 1080 at position 1088 corresponding with a controlled therapy ramp interval of about four minutes. At about position 1088, power input to the electrodes is reduced in the manner described, for example, in conjunction with FIG. 23 and, as represented by curve portion 1090 the reduced power input is provided with constant power control for about a one minute interval, for example, between the fourth and fifth minute to evoke the noted “thermal soak”.

For illustrative purposes, the temperature increase from the initial temperature of the tissue to be treated to the temperature necessary to achieve effective therapy is herein designated as ΔT, i.e. the temperature elevation. In the above example, as shown in FIG. 77, the initial temperature of the tissue, for instance, face tissue, is approximately 33° C. The temperature of the collagen shrinkage domain, 1080, extends from 65° C. to 75° C. Thus the minimum ΔT necessary to enter the collagen shrinkage domain is 32° C., and the maximum acceptable ΔT in this example is 42° C.

A number of substances have been identified that interact with the ECM of the dermis and alter the thermally responsive properties of the collagen fibers. As described herein, substances with such properties are termed “adjuvants.” A variety of such substances are known that function as temperature setpoint lowering adjuvants wherein utilization of such an adjuvant lowers the temperature elevation (ΔT) required to induce collagen shrinkage, i.e. lowers the thermal transformation temperature. The amount of reduction of the ΔT produced by a given concentration of a given adjuvant is identified herein as the ΔTₐ. It will be recognized by those skilled in the art of protein structural chemistry that the reduction in length of collagen fibers, i.e. shrinkage, is a result in part of an alteration of the physical structure of the molecular structure of the collagen fibers. The internal ultrastructure of collagen fibers, being comprised of tropocollagen molecules aggregated into collagen fibrils, and then aggregated further into even larger collagen fibers, is a result of complex interactions between the individual tropocollagen molecules, and between molecules associated with the collagen fibers, for example, elastin, and hyaluronan. The molecular forces of these interactions include covalent, ionic, disulphide, and hydrogen bonds; salt bridges; hydrophobic, hydrophilic and van der Waals forces. In the context of the invention, adjuvants are substances that are capable of inducing or assisting in the alteration of the physical arrangement of the molecules of the skin in order to induce, for instance shrinkage. With respect to collagen fibers, adjuvants are useful for altering the molecular forces holding collagen molecules in position, changing the conditions under which shrinkage of collagen can occur.

Protein molecules, such as collagen are maintained in a three dimensional arrangement by the above described molecular forces. The temperature of a molecule has a substantial effect on many of those molecular forces, particularly on relatively weaker forces such as hydrogen bonds. An increase in temperature may lead to thermal destabilization, i.e., melting, of the three dimensional structure of a protein. The temperature at which a structure melts is known as the thermal transformation temperature. In fact, irreversible denaturation of a protein, e.g., cooking, is a result of melting or otherwise disrupting the molecular forces maintaining the three dimensional structure of a protein to such an extent that that once heat is removed, the protein can no longer return to its initial three dimensional orientation. Collagen is stabilized in part by electrostatic interactions between and within collagen molecules, and in part by the stabilizing effect of other molecules serving to cement the molecules of the collagen fibers together. Stabilizing molecules may include proteins, polysaccharides (e.g., hyaluronan, chondroitin sulphate), and ions.

A persistent problem with existing methods of inducing collagen shrinkage that rely on heat is that there is a substantial risk of damaging and or killing adipose (fat layer) tissue underlying the dermis, resulting in deformation of the contours of the overlying tissues, with a substantial negative aesthetic effect. Higher temperatures or larger quantities of energy applied to the living cells of the dermis can moreover result in irreversible damage to those cells, such that stabilization of an altered collagen network cannot occur through neocollagenesis. Damage to the living cells of the dermis will negatively affect the ability of the dermis to respond to treatment through the wide variety of healing processes available to the skin tissue. Adjuvants that lower the ΔT required for shrinkage have the advantage that less total heat need be applied to the target tissue to induce shrinkage, thus limiting the amount of heat accumulating in the next adjacent subcutaneous tissue layer (hypodermis). Reducing the total energy application is expected to minimize tissue damage to the sensitive cells of the hypodermis, thereby limiting damage to the contour determining adipose cells.

One effect of adjuvants in relation to the invention is that certain biocompatible reagents have the effect of lowering the temperature required to begin disruption of certain molecular forces. In essence, adjuvants are capable of reducing the molecular forces stabilizing the ultrastructure of the skin, allowing a lower absolute temperature to induce shrinkage of the collagen network that determines the anatomy of the skin. Any substance that interferes with the molecular forces stabilizing collagen molecules and collagen fibers will exert an influence on the thermal transformation temperature (melting temperature). As collagen molecules melt, the three dimensional structure of collagen undergoes a transition from the triple helix structure to a random polypeptide coil. The temperature at which collagen shrinkage begins to occur is that point at which the molecular stabilizing forces are overcome by the disruptive forces of thermal transformation. Collagen fibers of the skin stabilized in the ECM by accessory proteins and compounds such as hyaluronan and chondroitin are typically stable up to a temperature of approximately 58° C. to 60° C., with thermal transformation and shrinkage occurring in a relatively narrow phase transition range of 60-70° C. Variations of this transition range are noted to occur in the aged (increasing the transition temperature) an in certain tissues (decreasing by 2-4° C. in tendon collagen). In effect the lower temperature limit of the collagen shrinkage domain is determined by the thermal transformation temperature of a particular collagen containing structure.

It will be recognized by those skilled in molecular biology that the thermal transformation temperature necessary to achieve a reduction in skin laxity may not entirely be determined by the thermal transformation temperature of collagen fibers, but may also be affected by a variety of other macromolecules present in the dermis, including other structural proteins such as elastin, fibronectin, heparin, carbohydrates such as hyaluronan and other molecules such as water and ions.

Referring again to FIG. 77, a hypothetical plot or curve 1092 showing desired electrode temperature with respect to therapy duration is presented wherein an adjuvant is used along with the implants. In the figure, a starting temperature is shown again to be, for example, 33° C. Above that temperature between about 53° C. and 63° C., when an adjuvant with a ΔTₐ of 12° C. is present, thereby lowering the ΔT necessary for thermal transformation by 12° C., there is established a collagen shrinkage domain represented generally at 1094. Shrinkage domain 1094 is seen to extend between the dashed line level 1096 corresponding with a collagen shrinkage threshold temperature of 53° C. and dashed line level 1098 corresponding with an upper limit level temperature of about 63° C. As represented previously at electrode temperature versus time curve portion 1096, variable power is applied to the bipolar electrodes as a ramp control commencing at the noted 33° C. and reaching the upper limit of 63° C. within domain 1094 at position 1099 corresponding with a controlled therapy ramp interval of about four minutes. At about position 1099, power input to the electrodes is reduced in the manner described, for example, in conjunction with FIG. 23 and, as represented by curve portion 1100 the reduced power input is provided with constant power control for about a one minute interval, for example, between the fourth and fifth minute to evoke the previously noted “thermal soak”.

Substances exhibiting the properties desirable for lowering the ΔT include enzymes such as hyaluronidase collagenase and lysozyme; compounds that destabilize salt bridges, such as beta-naphtalene sulphuric acid; each of which is expected to reduce the ΔT by 10-12° C., and substances that interfere with hydrogen bonding and other electrostatic interactions, such as ionic solutions, such as calcium chloride or sodium chloride; detergents (a substance that alters electrostatic interactions between water and other substances), such as sodium dodecyl sulphate, glycerylmonolaurate, cationic surfactants, or N,N, dialkyl alkanolamines (i.e. N,N-diethylethanolamine); lipophilic substances (lipophiles) including steroids, such as dehydroepiandrosterone, and oily substances such as eicosapentanoic acid; organic denaturants, such as urea; denaturing solvents, such as alcohol, ethanol, isopropanol, acetone, ether, dimethylsulfoxide (DMSO) or methylsulfonylmethane; and acidic or basic solutions. The adjuvants that interfere with hydrogen bonding and other electrostatic interactions may reduce the ΔT for the shrinkage transition by as much as 40° C. depending on the concentration and composition of the substances administered. The ΔTₐ of a particular adjuvant in use will be dependent on the chemical properties of the adjuvant and the concentration of adjuvant administered to the patient. For enzymatic adjuvants such as hyaluronidase, the ΔTₐ is also dependent on the specific activity of the delivered enzyme adjuvant in the dermis environment.

Adjuvants suitable for use would desirably be compatible with established medical protocols and be safe for use in human patients. Adjuvants should be capable of rapidly infiltrating the targeted skin tissue, should cause minimal negative side effects, such as causing excess inflammation, and should preferably persist for the duration of the procedure. Suitable adjuvants may be, for instance, combined with local anesthetics used during treatment, be injectable alone or in combination with other reagents, be heat releaseable from the implants of the invention, or be capable of entering the targeted tissue following topical application to the skin surface. Certain large drug molecules, such as enzymes functioning as adjuvants according to the invention may be drawn into the target dermal tissue through iontophoresis (electric current driving charged molecules into the target tissues) The exact mode administration of adjuvants will be dependent on the particular adjuvant employed.

In a preferred embodiment, the thermal transition temperature lowering adjuvant is present in highest concentrations in the tissues of the dermis. For highest efficacy, a concentration gradient is established, wherein the adjuvant is at a higher concentration in the dermis that in the hypodermis. A transdermal route of administration is one preferred mode of administration, as will occur with certain topical adjuvants. For adjuvants that are applied topically to the surface of the skin, for instance as a pomade, as the adjuvant either diffuses or is driven across the epidermis, and passes into the dermis, a concentration gradient is established wherein the adjuvant concentration is higher in the dermis than in the hypodermis. Because the collagen matrix is much more prevalent in the dermis than in the epidermis, presence of the adjuvant in the epidermis is expected to be without negative effect. Certain adjuvants, for instance, enzymes with collagen binding activity, would be expected to accumulate in the dermal tissue.

A variety of methods are known wherein drugs are delivered to the patient transdermally, i.e. percutaneously, through the outer surface of the skin. A variety of formulations are available that enhance the percutaneous absorption of active agents. These formulations may rely on modification of the active agent, or the vehicle or solvent carrying that agent. Such formulations may include solvents such as methylsulfonylmethane, skin penetration enhancers such as glycerylmonolaurate, cationic surfactants, and N,N, dialkyl alkanolamines such as N,N-diethylethanolamine, steroids, such as dehydroepiandrosterone, and oily substances such as eicosapentanoic acid. For further discussion of enhancers of transdermal delivery of active agents, for instance adjuvants according to the invention, see: U.S. Pat. No. 6,787,152 to Kirby et al., issued Sep. 7, 2004; and U.S. Pat. No. 5,853,755 to Foldvari, issued Dec. 29, 1998.

When adjuvants are injected, it is preferable that they be deposited as close to the dermis as practicable, preferably, intradermally. Because the dermis is relatively thin, and difficult to penetrate with hypodermic needles, the invention is also embodied in adjuvants that are delivered subdermally, or at the interface between the dermis and the next adjacent subcutaneous tissue (hypodermis or adipose tissues underlying the dermis). Even to the extent that adjuvants are delivered into the adipose tissue of the hypodermis, because the hypodermis is typically very thick compared to the dermis, a concentration gradient will develop, wherein the adjuvant will diffuse quickly into the dermis, and fully equilibrate with the dermal tissue, before the adjuvant has fully equilibrated with the hypodermis.

In a further embodiment, the implants carry a surface coating of adjuvant that is released into the dermis upon activation of the implant. It is an advantage of the invention when utilizing ΔT lowering adjuvants that the implants are placed very near the location where adjuvants can provide the most benefit. A number of compositions are known in the art that can be released from an implant by heating of the implant. For example, the upper, or dermis facing, surface of the implant can be coated with microencapsulated adjuvant, for instance hyaluronan. Once a preliminary heating of the implant begins, the encapsulated adjuvant is released, and immediately begins diffusing into the dermis tissue, as the implant is already in place at the interface between the dermis and hypodermis. As the adjuvant diffuses through the dermis, a concentration gradient develops wherein the adjuvant is at the greatest concentration in the dermis, with reduced concentrations in the epidermis and hypodermis. Following this preliminary heating, regular ramp up to a lowered setpoint temperature may be carried out. As described previously, while it is not a requirement that the adjuvant be at greatest concentration in the dermis (for instance, if the adjuvant is applied topically to the skin surface, it is considered an advantage to for the adjuvant to be at the greatest concentration in the tissue layer wherein adjuvant activity is needed.

In a further embodiment of implant delivery of the adjuvant, the adjuvant is encapsulated in liposomes and suspended in a compatible vehicle. The surfaces of the implant to be inserted into the patient are then coated with the liposome/vehicle composition. When the implant is inserted into the tissue of the patient, the vehicle coating, preferably moderately water soluble and biologically inert, prevents the adjuvant from being displaced from the implant surface for the period of time necessary for insertion. Once the implant is activated on the noted preliminary basis, the dermis facing upper surface of the implant is heated and the liposomes encapsulating the adjuvant are induced by heat to release the adjuvant. The adjuvant may alternatively be released from hybrid implants by brief preliminary heating utilizing the resistor heating component of implants with a hybrid architecture, as described in connection with FIGS. 63-64. Different compositions of liposomes are useful for providing release of the adjuvant at a particular temperature range. Similarly, the vehicle binding the adjuvant encapsulating liposomes to the implant can be chosen so that the vehicle does not release the liposomes themselves unless a desired temperature has been reached. In this manner the release of adjuvant from an implant surface may be configured so that the adjuvant is released in a directional manner, even though the entire implant surface is coated with an adjuvant composition. Those skilled in the art will recognize that a variety of heat releaseable encapsulating systems are available for use with the invention. Further discourse on the composition of liposomes is available by referring to U.S. Pat. No. 5,853,755 (supra).

The following discourse specifically describes certain embodiments of specific adjuvants that are useful. Artisans will recognize that other substances known in the art to have similar effects will be useful as adjuvants, and thus, the following embodiments should not be considered as limiting.

Hyaluronidase is an enzyme that cleaves glycosidic bonds of hyaluronan, depolymerizing it and, converting highly viscous polymerized hyaluronan into a watery fluid. A similar effect is reported on other acid mucopolysaccharides, such as chrondroitin sulphate. Hyaluronidase is commercially available from a number of suppliers (e.g., Hyalase, C.P. Pharmaceuticals, Red Willow Rd. Wrexham, Clwydd, U.K.; Hylenex, Halozyme Therapeutics (human recombinant form); Vitrase, (purified ovine tissue derived form) ISTA Pharmaceuticals; Amphadase, Amphastar Pharmeceuticals (purified bovine tissue derived)).

Hyaluronidase modifies the permeability of connective tissue following hydrolysis of hyaluronan. As one of the principal viscous polysaccharides of connective tissue and skin, hyaluronan in gel form, is one of the chief ingredients of the tissue cement, offering resistance to the diffusion of liquids through tissue. One effect of hyaluronidase is to increase the rate of diffusion of small molecules through the ECM, and presumably to decrease the melting temperature of collagen fibers necessary to induce shrinkage. Hyaluronidase has a similar lytic effect on related molecules such as chondroitin sulphate. Hyaluronidase enhances the diffusion of substances injected subcutaneously, provided local interstitial pressure is adequate to provide the necessary mechanical impulse. The rate of diffusion of injected substances is generally proportionate to the dose of Hyaluronidase administered, and the extent of diffusion is generally proportionate to the volume of solution administered. The addition of hyaluronidase to a collagen shrinkage protocol results in a reduction of the ΔT required to induce 20% collagen shrinkage by about 12° C. Review of pharmacological literature reveals that doses of hyaluronidase in the range of 50-1500 units are used in the treatment of hematomas and tissue edema. Thus, local injection of 1500 IU hyaluronidase in 10 ml vehicle into the target tissue is predicted to reduce the temperature necessary to accomplish 20% shrinkage of collagen length from about 63° C. to about 53° C. For multiple injection sites 100 IU hyaluronidase in 2 ml of alkalinized normal saline or 200 IU/ml are expected to be similarly effective as an adjuvant. The manufacturer's recommendations for Vitrase indicate that 50-300 IU of Vitrase per injection are expected to exert the adjuvant effect. It should be noted that use of salimasa vehicle for delivery of adjventson anesthesia may be contradicted where introduction of excess electrolytes would interfere with operation of the implants.

Hyaluronidase has been used in clinical settings as an adjunct to local anesthesia for many years, without significant negative side effects, and is thus believed to be readily adaptable for use in practicing the invention. When used as an adjunct to local anesthesia, 150 IU of hyaluronidase are mixed with a 50 ml volume of vehicle that includes the local anesthetic. A similar quantity of hyaluronidase is expected to be effective reducing the ΔT for effecting shrinkage by approximately 10° C., with or without the addition of anesthetic. When hyauronidase is injected intradermally or peridermally, the dermal barrier removed by hyaluronidase activity persists in adult humans for at least 24 hours, with the permeabilization of the dermal tissue being inversely related to the dosage of enzyme delivered (in the range of administered doses of 20, 2, 0.2, 0.02, and 0.002 units per mL. The dermis is predicted to be restored in all treated areas 48 hours after hyaluronidase administration. Additional background on the activity of hyaluronidase is available by referring to the following publications (and the references cited therein):
18. Lewis-Smith, P. A., “Adjunctive use of hyaluronidase in local anesthesia” Brit. J. Plastic Surgery, 39: 554-558 (1986).
19. Clark, L. E., and Mellette, J. R., “The Use of Hyaluronidase as an Adjunct to Surgical Procedures” J. Dermatol., Surg. Oncol., 20: 842-844 (1994).
20. Nathan, N., et al., “The Role of Hyaluronidase on Lidocaine and Bupivacaine Pharmaco Kinetics After Peribulbar Blockade” Anesth Analg., 82: 1060-1064 (1996).

See also U.S. Pat. No. 6,193,963 to Stern, et al., issued Feb. 27, 2001.

Lysozyme is an enzyme capable of reducing the cementing action of ECM compounds such as chondroitin sulphate. Lysozyme (aka muramidase hydrochloride) has the advantage that it is a naturally occurring enzyme; relatively small in size (14 kD), allowing rapid movement through the ECM; and is typically well tolerated by human patients. A topical preparation of lysozyme, as a pomade of lysozyme is available (Murazyme, Asta Medica, Brazil; Murazyme, Grunenthal, Belgium, Biotene with calcium, Laclede, U.S.). The addition of lysozyme as an adjuvant to a collagen shrinkage protocol results in a reduction of the ΔT required to induce 20% collagen shrinkage by about 10-12° C. Additional background on the use of lysozyme to lower the ΔT for collagen shrinkage is available. See for instance, U.S. Pat. No. 5,484,432 to Sand, issued Jan. 16, 1996.

Those skilled in the art will recognize that a variety of adjuvants that reduce the stability of the collagen fiber, tropocollagen, and or substances that serve to cement these structures are adaptable for use with the heater implants of the invention. Adjuvant ingredients may include agents such as solvents, such as dimethylsulfoxide (DMSO), monomethylsulfoxide, polymethylsulfonate (PMSF), methylsulfonylmethane, alcohol, ethanol, ether, diethylether, and propylene glycol. Certain solvents, such as DMSO, are known to lead to the disruption of collagen fibers, and collagen turnover. When DMSO is delivered to patients with scleroderma, a condition that exhibits an overproduction of collagen and scar tissue as a symptom, an increase of excretion of hydroxyproline, a constituent of collagen is noted. This is believed to due to increased breakdown of collagen. Solvents that will alter the hydrogen bonding interactions of collagen fibers, such as DMSO and ethanol are predicted to reduce the ΔT necessary to reach the thermal transition temperature of collagen fibers, with the reduction of ΔT being expected to be relative to the alteration of the hydrophilicity of the collagen environment by the solvent. Small diffusible solvents such as DMSO and ethanol offer the further advantage of being able to rapidly penetrate the epidermis and reach the dermis tissue, while being generally safe for use in human patients.

In a further embodiment, adjuvants may be used in combination with one another, in a manner that either further lowers the ΔT either synergistically or additively. Combining adjuvants provides a means to utilize a particular adjuvant to achieve its optimal effect, and when combined with a second adjuvant, further lower the heating necessary to achieve the desired shrinkage, while avoiding adverse side effects associated with higher doses of a particular adjuvant.

Where three of more implants are utilized in a given therapy session, a discussion has been provided, for instance, in conjunction with FIGS. 62A and 62B with respect to current flux path distribution of energy among border implants and inwardly disposed implants. A 50% duty cycle with respect to such implant groupings has been discussed. In consequence of that rationalization, a sequence of ex vivo animal (pig) studies was carried out employing electrodes as described at 40 in connection with FIGS. 4 and 5. An end view schematic representation of the testing undertaken is shown in FIG. 78. Looking to that figure, epidermis is shown in general at 1104; dermis at 1106 and the next adjacent subcutaneous tissue or fat layer at 1108. Three single electrode implants represented generally at 1110-1112 were located at the interface 1114 between dermis 1106 and fat layer 1108. As discussed in connection with FIGS. 4 and 5, these implants were configured with platinum electrodes having a width of 0.130 inch, a length of 1.0 inch and a thickness of 0.001 inch. The electrodes were mounted upon a polymeric support and thermal barrier having a width of 3 millimeters. Centrally disposed and located at the middle of each electrode was a thermocouple shown respectively at 1116-1118. As labeled on the drawing, the electrodes of implants 1110-1112 were spaced apart center-to-center 15 millimeters. This means for each bipolar electrode pair, the outside edge spacing was 18 millimeters and the inside edge mutually adjacent edge spacing was 12 millimeters. The electrodes of implants 1110 and 1111 were coupled to a prototype radiofrequency generator, RF 1 as represented at block 1120 and lines 1122 and 1124. In similar fashion paired electrode 1111 and border electrode 1112 were coupled to a second prototype radiofrequency generator, RF 2 as represented at block 1126 and lines 1128 and 1130. With the arrangement, no cross current would be present between generators RF 1 and RF 2 and they were operated under a constant power of seven watts. Rather than a duty cycle-type of powering, the supplied power was continuous. As this occurred, the temperatures at thermocouples 1116-1118 as labeled respectively T_{A}, T_{B}, and T_{C}, were monitored. As current flux paths were created as represented in general at 1132 and 1134, temperatures T_{A}, T_{B}, and T_{C}, remained essentially the same. It is opined that this unexpected phenomena is due at least in part to the positioning and spacing described above. Enough tests were carried out to show that the shared electrode as at 1111 can be powered in combination with two border electrodes simultaneously without experiencing an undesired thermal excursion.

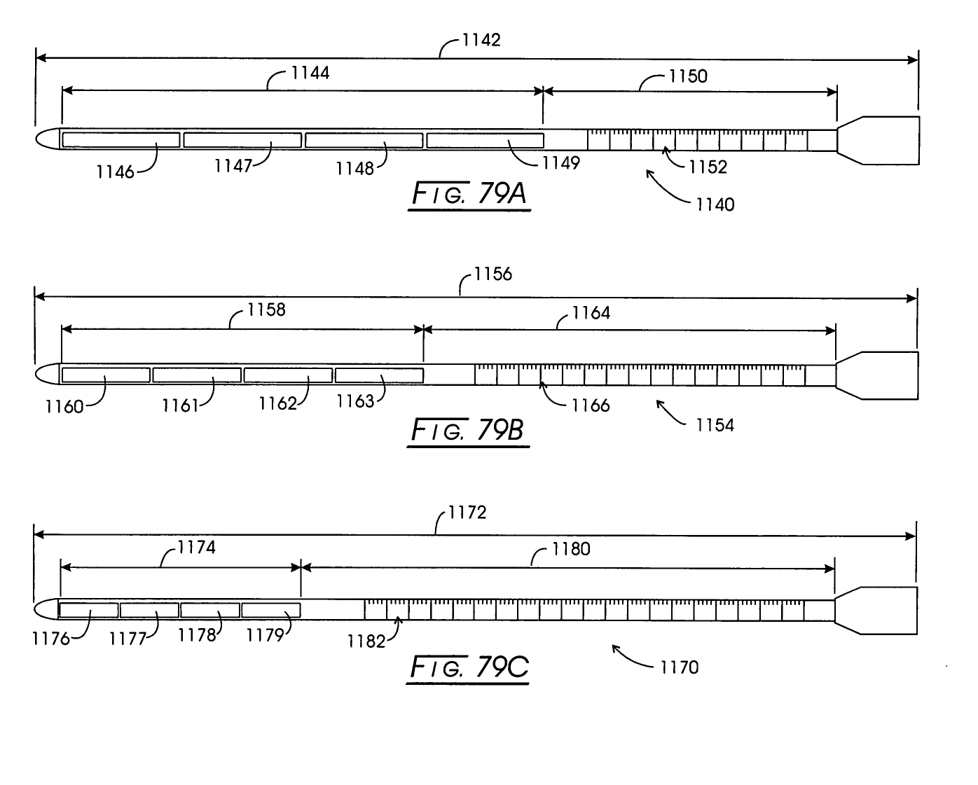

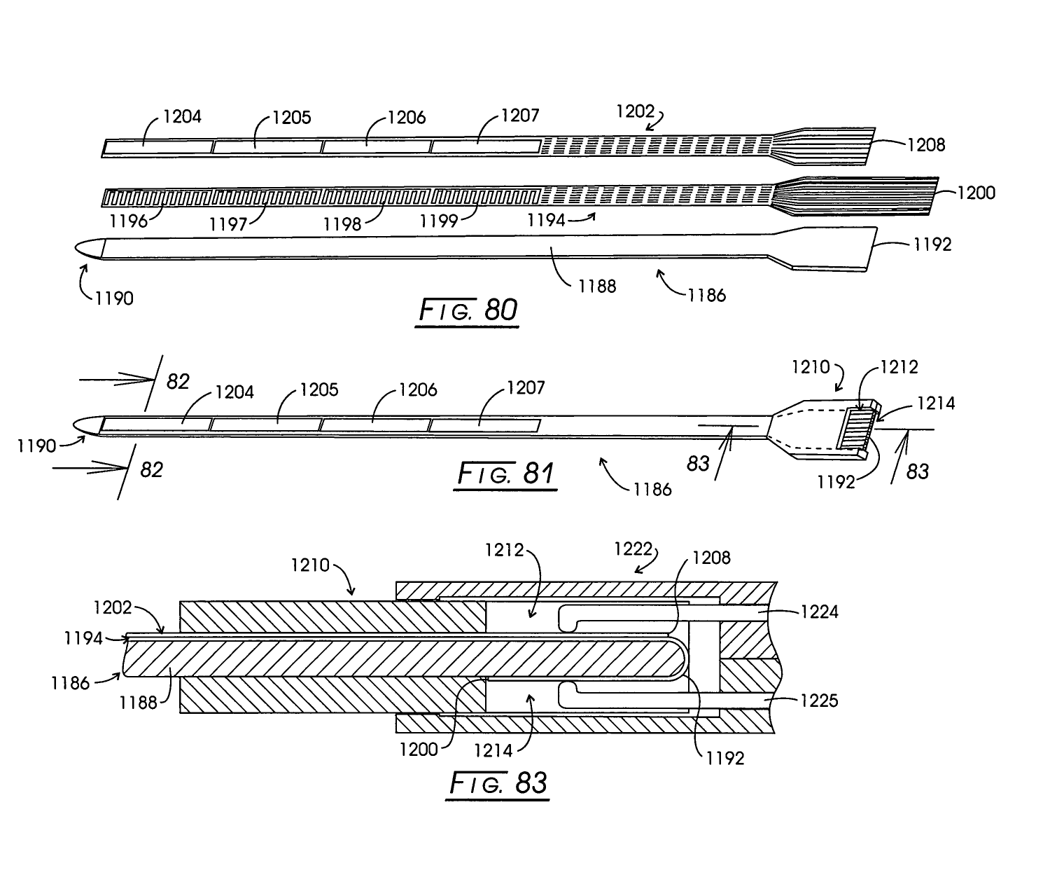

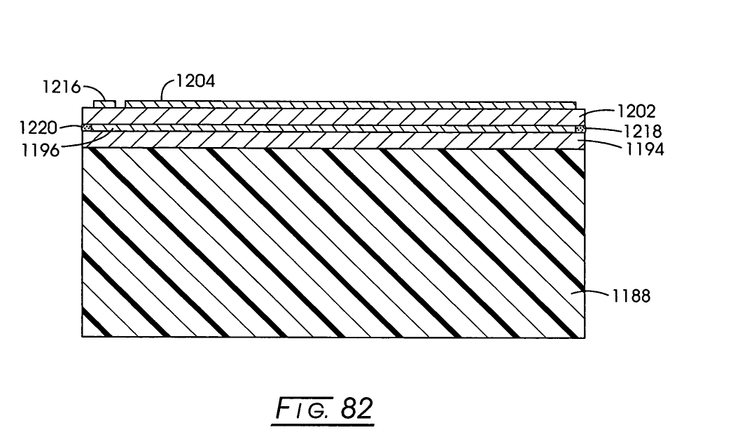

In the course of development of the instant implants and method, it was determined that the overall length of the implants utilized should be a fixed value, for instance, 7.75 inches and that the active or heating region within that constant implant length should vary but be formed with a consistent, identical number of electrodes and associated temperature sensing resistor segments. By thus standardizing the number of electrodes, for example, four, the associated control system may be more simply configured to consistently perform in conjunction with that number of electrodes. FIGS. 79A-79C combine to illustrate this standardization approach in structuring the implants which developers have referred to as “wands”. In FIG. 79A, one version of such an implant is represented in general at 1140 and is labeled at dimension arrow 1142 as having a fixed length. As noted above, that length may, for example, be 7.75 inches. Within this fixed length there is a heating region represented by and labeled at dimension arrow 1144 which encompasses four electrodes 1146-1149. The heating region length at arrow 1144 may, for example, be 3.2 inches and the length of the electrodes 1146-1149 may be, for example, 15 millimeters. From the heating region arrow 1144 there extends a non-heating region represented at dimension arrow 1150 which supports no electrodes and extends that length of the implant which remains 3 millimeters in width. Because the implants may be inserted at the dermis-hypodermis interface from aesthetically elected entrance locations, positioning or insertion indicia as represented generally at 1152 may be imprinted along the non-heating region and visually related to the entrance incision location. Indicia 1152 are somewhat similar to the distance marking indicia on catheters.

Looking to FIG. 79B, a next version of a system implant is represented generally at 1154. Implant 1154 is configured with a fixed consistent length corresponding with that of implant 1140, i.e., 7.75 inches as represented at dimension arrow 1156. The heating region for implant 1154 is represented at dimension arrow 1158 and will be shorter than the heating region of implant 1140, for example, being about 2.4 inches in length. However, within the heating region remain a consistent four electrodes 1160-1163. Those electrodes may, for example, have a length of 12 millimeters. Extending rearwardly from the heating region, as before, is a non-heating region represented by dimension arrow 1164. This non-heating region may be observed to be lengthier than the corresponding non-heating region of implant 1140. As before, positioning or insertion indicia as represented generally at 1166 may be provided along the non-heating region.

Referring to FIG. 79C, a third version for the implant is represented in general at 1170. Implant 1170 has the noted fixed length which is consistent with that of implants 1140 and 1154 as represented at dimension arrow 1172. Implant 1170 is configured with a heating region of about 1.6 inches in length as represented at dimension arrow 1174. As before, the heating region incorporates four RF electrodes, 1176-1179. These electrodes may have a length, for example, of 8 millimeters. The non-heating region for implant 1170 is more elongate as represented by dimension arrow 1180. This non-heating region incorporates positioning or insertion indicia as represented generally at 1182.

A custom design connector guide has been described in connection with FIGS. 41-44 as a component of the implant. Because of the offset location of connection with leads from the resistor segments with a cable connector (FIG. 44), the cable connector itself also is custom fabricated. However, the implants of the invention may be designed to perform in conjunction with commercially available or “off the shelf” cable connectors. One such connector is a type MECI-108-02-S-D-RAI-SL marketed by SAMTEC, Inc. of New Albany, Ind. With that connector, over and under contacts are provided, however, they are in mutual alignment.

Referring to FIG. 80, this revised implant is represented generally at 1186 in exploded fashion. Device 1186 is configured with a support and thermal barrier 1188. Formed of the earlier-described polyetheramide, thermal barrier 1188 extends from a leading end represented generally at 1190 to a trailing end 1192. Note that the leading end 1190 is configured somewhat as a “sled” to facilitate insertion of implant 1186 within a heating channel. The thickness of component 1188 is now 0.060 inch. In the earlier embodiments, the flexible circuit carried RF electrodes on an outward surface and temperature sensing resistor segments on the opposite surface. Upon implant 1186, a separate polyamide flexible circuit support or substrate is provided to support these temperature sensing resistors. In this regard, this separate polyamide circuit support is shown in general at 1194 carrying four resistor segments 1196-1199, the four-point leads to which extend rearwardly to end 1200. Formed of a polyamide (Kapton) with a thickness of 0.001 inch, the flexible circuit or substrate 1194 is adhesively adhered to the upward or support surface of support 1188. However, the portion of the component 1194 extending to end 1200 extends over trailing end 1192 of support 1188. Shown aligned with and extending over circuit 1194 is a second circuit support represented generally at 1202. Component 1202 carries four gold on copper RF electrodes 1204-1207 from which extend a corresponding four leads which terminate at an end edge 1208. Note that end 1200 of component 1194 extends beyond edge 1208. Component 1202 also is formed of a 0.001 inch thick polyamide (Kapton) and is adhesively secured over component 1194 in a manner wherein the resistor segments 1196-1194 and the lead components are encapsulated and thus protected from body fluids and the like.

Looking to FIG. 81, implant 1186 is shown assembled with a polymeric connector guide identified generally at 1210 having an upper slot shown generally at 1212 and a lower slot represented generally at 1214. Slots 1212 and 1214 provide access for the contacts of a cable connector.

Referring to FIG. 82, a sectional view of a forward portion of implant 1186 is presented. In the figure, thermal barrier and support 1188 is shown supporting flexible circuit component 1194 which, in turn, is shown supporting temperature sensing resistor segment 1196. Adhesively secured over the copper resistor component as at 1196, is component 1202 shown carrying gold-plated copper electrode 1204 and a section of a lead therefrom 1216. Component 1194 is somewhat encapsulated through the use of a medical grade adhesive, two components of which are seen at 1218 and 1220.

Referring to FIG. 83, implant 1186 is shown in engagement with a polymeric cable connector 1222. Note that the rearward portion of component 1194 has been wrapped around end 1192 of support and thermal barrier 1188. Thus, leads are available to cantilevered connector contacts, two of which are shown at 1224 and 1225.

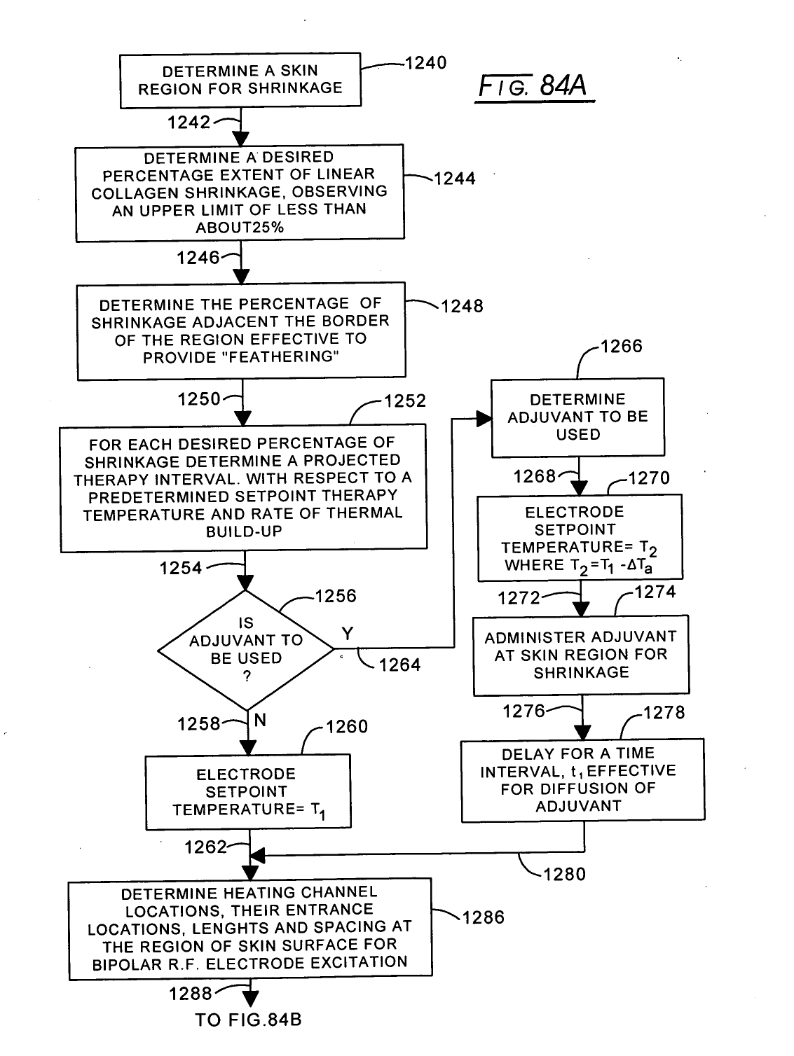

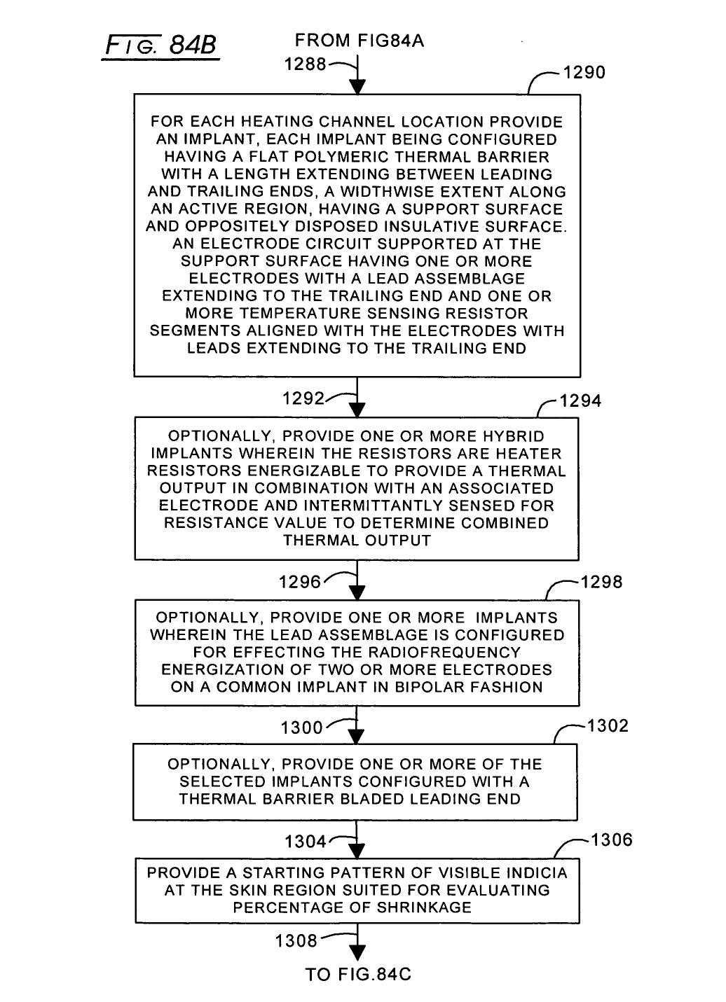

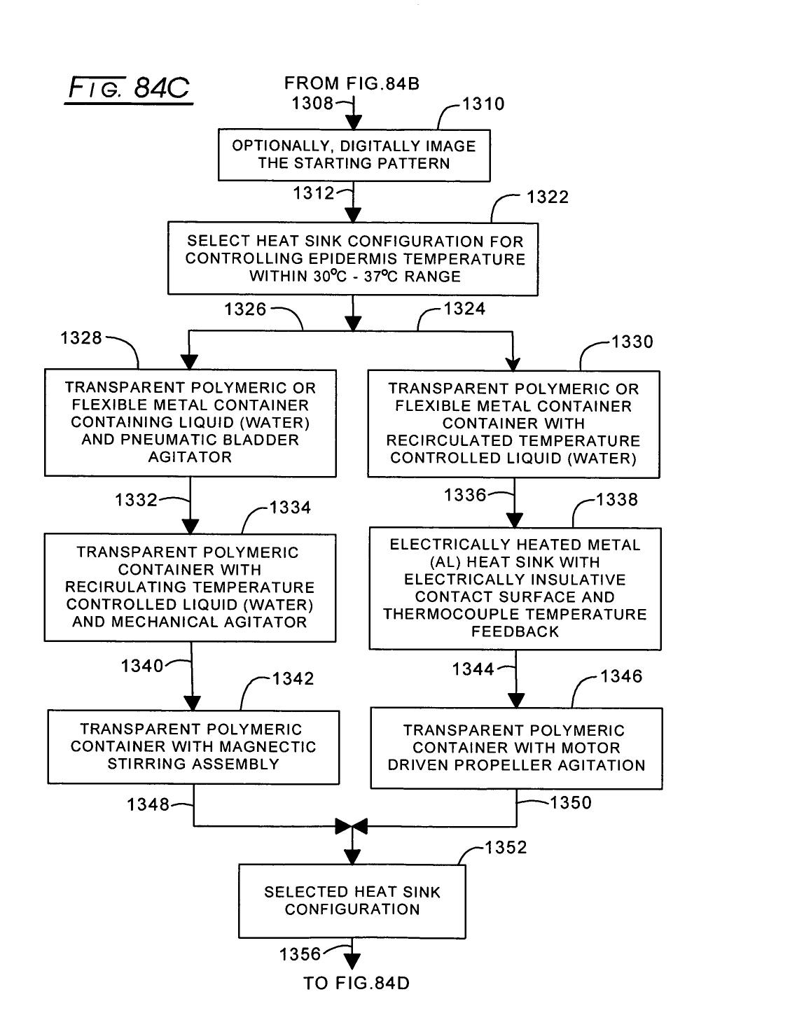

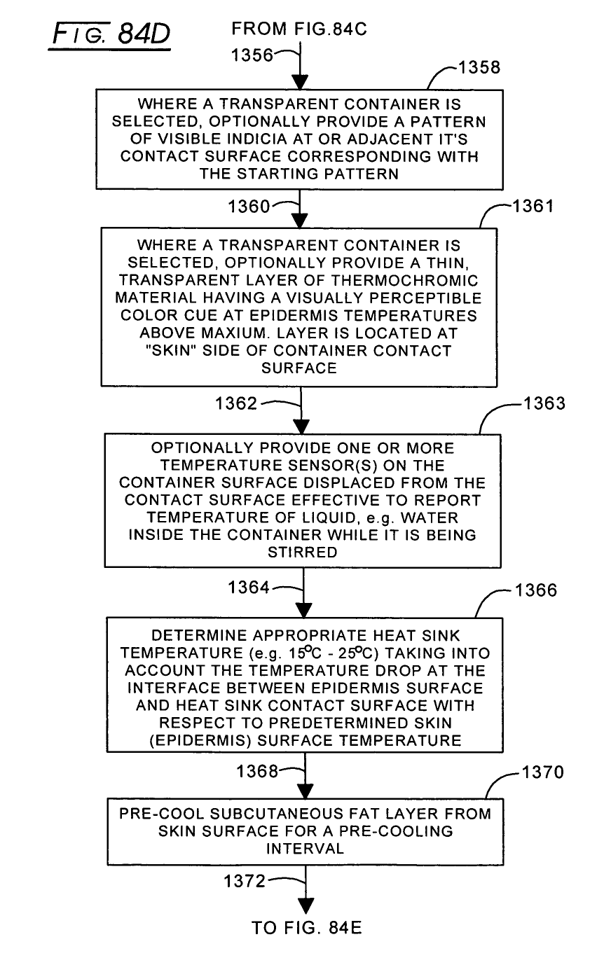

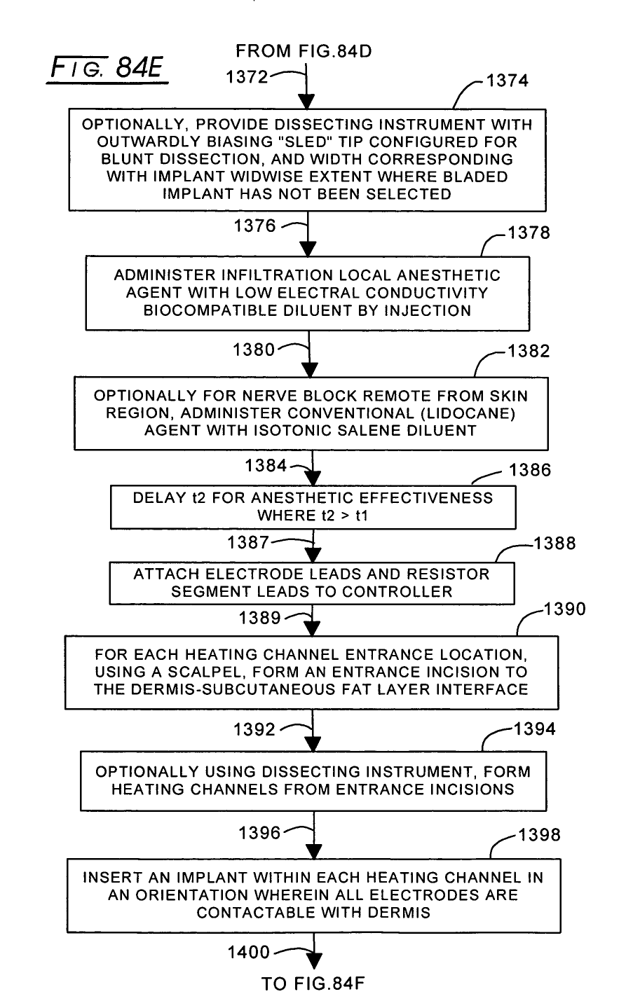

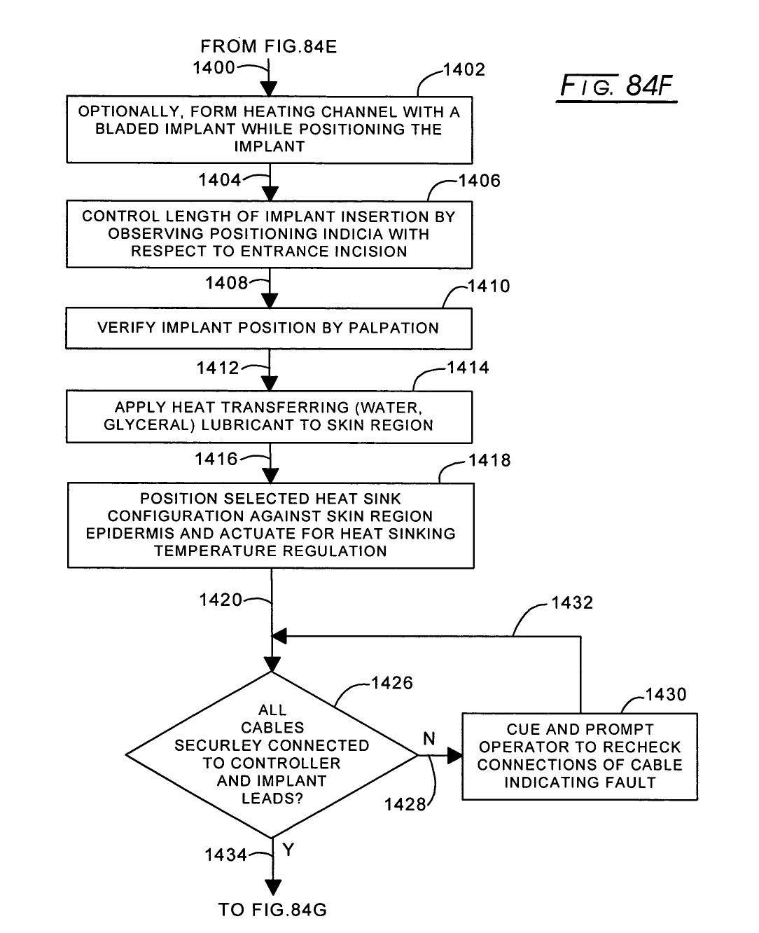

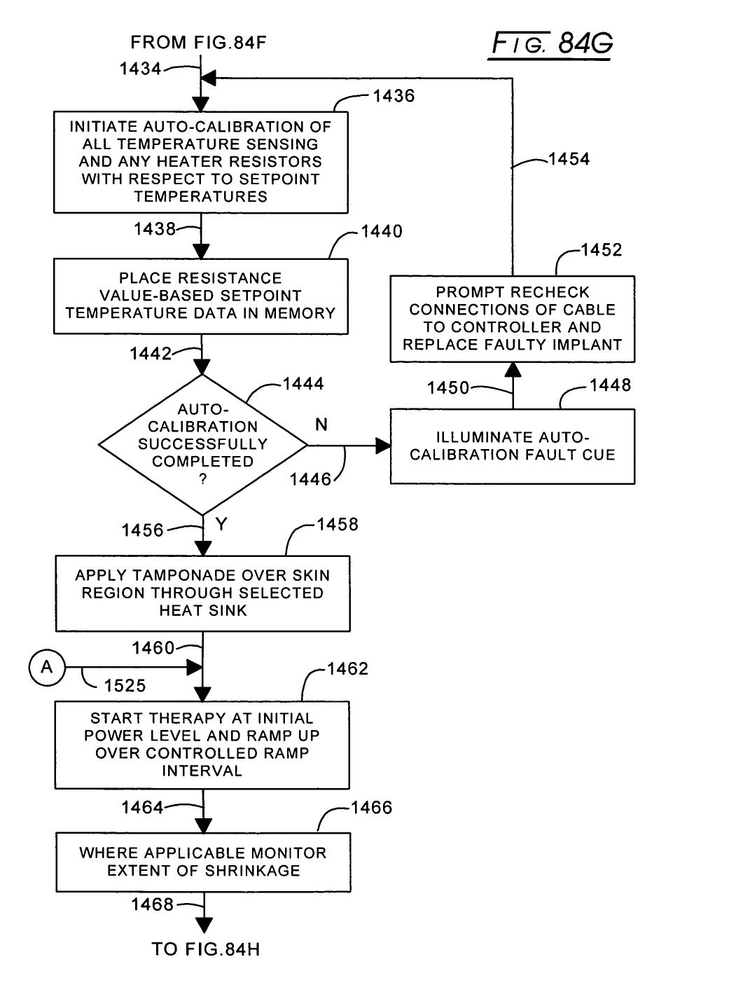

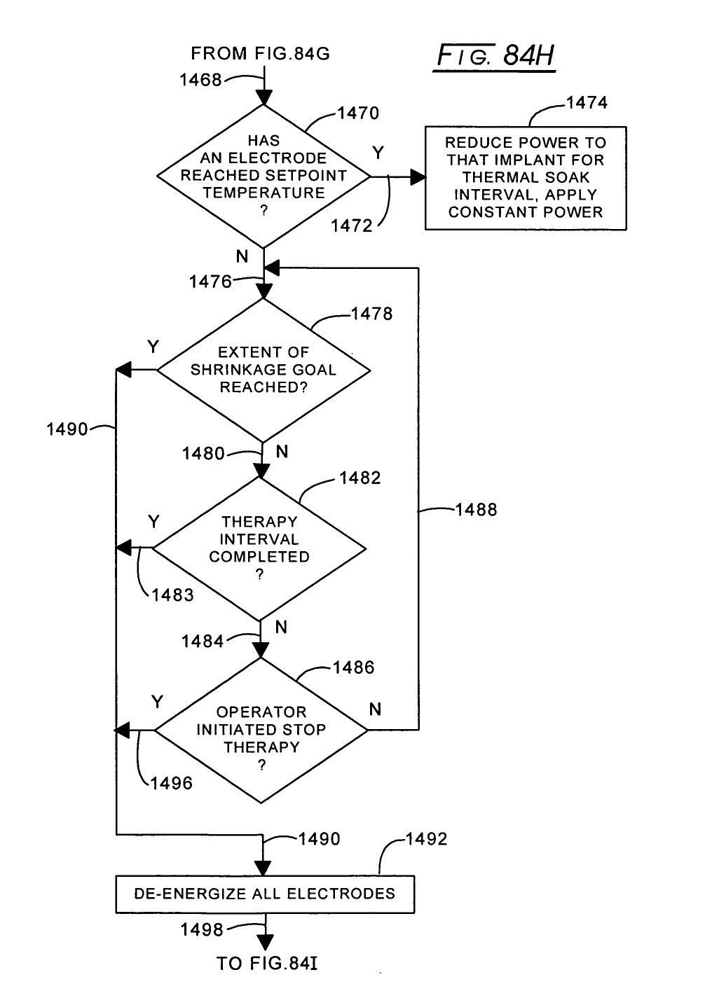

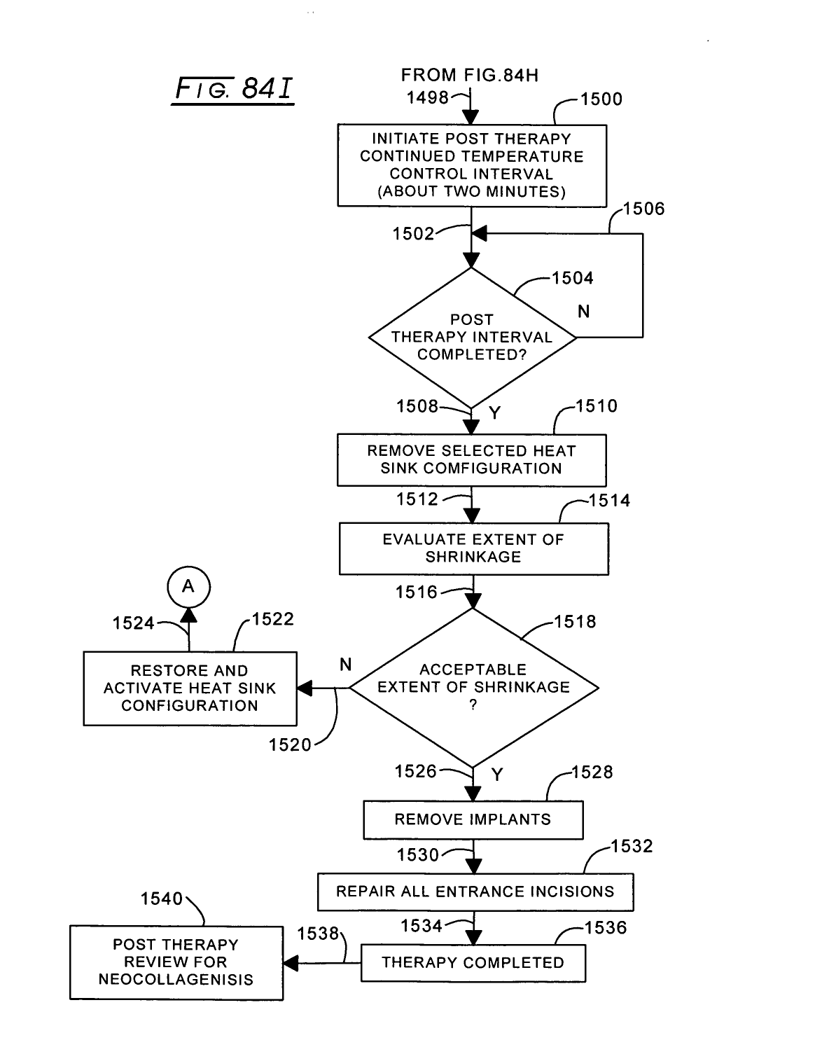

FIGS. 84A-84I combine as labeled thereon to provide a flowchart describing the method of the invention. At the commencement of the procedure, the clinician determines that skin region suited for shrinkage as indicated at block 1240. In correspondence with this determination, as represented at line 1242 and block 1244, a determination is made as to the desired percentage extent of linear collagen shrinkage. In this regard, an upper limit of less than about 25% shrinkage is recommended. Line 1246 extends from block 1244 to the determination at block 1248 wherein consideration is made as to the amount of shrinkage to be provided at the borders of the skin region to provide a form of “feathering”. Once the parameters of shrinkage are determined, then as represented at line 1250 and block 1252 a therapy interval can be projected or estimated. That interval will be determined with respect to a predetermined setpoint therapy temperature, rate of thermal build-up and soak interval as discussed in connection with FIG. 77. The quantification of therapy intervals has been discussed above in connection with equation (1) and publication 16. These determinations also are predicated upon whether a temperature setpoint lowering adjuvant is to be used in conjunction with the heating of the skin region for shrinkage, for instance, hyaluronidase may be topically administered to the surface of the skin region. Accordingly, as represented at line 1254 and block 1256, a query is posed as to whether adjuvant is to be used. If it is not to be used, then as represented at line 1258 and block 1260 the setpoint temperature (electrode) is established as T₁. This corresponds with horizontal dashed line 1094 in FIG. 77. The method then continues as represented at line 1262.

Use of such adjuvant is highly beneficial in terms of providing thermal protection to both the next adjacent subcutaneous tissue or fat layer as well as to the epidermis, with the lower temperature collagen shrinkage domain being developed by delivering adjuvant to the skin region targeted for shrinkage. Administration of adjuvant may be carried out, for instance, by topically applying it over the targeted skin surface, or by delivering adjuvant from the surface of the implant. Where the query posed at block 1256 results in an affirmative determination that an adjuvant is to be used, then as represented at line 1264 and block 1266, the type and quantity of adjuvant and the adjuvant delivery system are determined. As represented at line 1268 and block 1270 the setpoint temperature is established as T₂, wherein the basic setpoint temperature T₁, is diminished to the extent of ΔTₐ, where ΔTₐ is equal to the reduction of the ΔT necessary to reach the collagen shrinkage domain as shown in FIG. 77, based on the type and quantity of adjuvant to be delivered. For the example of hyaluronidase, ΔTₐ, the reduction of ΔT, is about 10° C. to 12° C., and thus T₂ is 10° C. to 12° C. less than T₁. This setpoint T₂, is described in connection with horizontal dashed line 1096 in FIG. 77 (for hyaluronidase).

Whether the adjuvant chosen at block 1266 is to be topically applied or otherwise it is administered to the skin region targeted for shrinkage as represented at line 1272 and block 1274. After administration of the adjuvant, as represented at line 1276 and block 1278, a delay for time interval t₁, ensues of time length effective for diffusion of the adjuvant, for example, through the stratum corneum and remaining epidermis and into the dermis, a concentration gradient being involved which delivers adjuvant to the dermis and including the time length necessary for the adjuvant to lower ΔT. Following the delay interval t₁, any excess adjuvant resulting from topical application may be removed from the skin surface. In this regard, the adjuvant may be incorporated in a cream carrier. Removal of the excess adjuvant also clears the skin surface for providing a starting pattern of visible indicia such as dots. However, the excess adjuvant at the skin surface may be permitted to remain and function as a heat transfer and lubricating medium.

When the adjuvant chosen at block 1266 is to be an implant delivered one, the adjuvant is activated by heating of the implant for a time interval of length effective for release of the adjuvant from the implant. A delay then ensues for a time length effective for diffusion of the adjuvant into the dermis, a concentration gradient being involved which delivers adjuvant to the dermis, and including the time length necessary for the adjuvant to lower the ΔT. The adjuvant application features described with respect to transdermal or implant delivered adjuvants also may be carried out when utilizing other adjuvants and delivery systems. When employing other adjuvant delivery methods, such as iontophoretic delivery, the adjuvant may be applied to the skin surface, and then drawn into the dermis by activation of an appropriate electric field. Delay periods necessary for activity of the delivered adjuvant are familiar to those employing known methods in dermatologic fields, including for instance, local anesthesia.

The program then as represented at line 1280 returns to line 1262. Line 1262 is seen to extend to block 1286 providing for a determination of heating channel locations including their entrance locations, lengths and generally parallel spacing. Next, as represented at line 1288 and block 1290 an implant is provided for each channel location. In general, these implants may be structured as described in connection with FIGS. 37-39 and may be further implemented as described in connection with FIGS. 67-70, 79A-79C and 80-83. As represented at line 1292 and block 1294, a clinician optionally may elect to utilize one or more hybrid implants wherein the resistor segments not only function as temperature sensors but also as heating elements. Such hybrid devices have been described in connection with FIGS. 63 and 64 with respect to the resistor segment pattern, it also being recalled that FIGS. 39 and 40 were revisited in this regard to indicate that a four-point lead structuring can be used with the combined heating and temperature sensing resistor segments. When the implant is configured to carry adjuvant the hybrid form (FIGS. 63, 64) may be employed. With this arrangement the resistor segments may initially be heated to release adjuvant, following which the RF electrodes may be excited for effecting collagen shrinkage. From block 1294, line 1296 extends to block 1298. At that block, an optional provision is made for electing to utilize a bipolar electrode assembly mounted upon a single implant substrate. Such an implant has been described in connection with FIGS. 73 and 74. As another option, as represented at line 1300 and block 1302 the implant can be a bladed one as described in conjunction with FIGS. 67-70. Line 1304 extends from block 1302 to block 1306. The latter block describes the provision of a starting pattern of visible indicia at the surface of the skin suited for evaluating the percentage of shrinkage developed. Such indicia has been described in connection with FIGS. 56-58. The pattern may be developed with a template and, as represented at line 1308 and block 1310, a digital image of the starting pattern may be provided. As represented at line 1312 and block 1322 a heat sink configuration is selected for controlling the temperature at the epidermis surface to reside within a range of about 30° C. not to exceed 37° C.

A variety of heat sink configurations have been described. Lines 1324 and 1326 extend to blocks 1328 and 1330. Block 1328 describes a transparent polymeric conformal bag-like container incorporating a pulsating pneumatic bladder as described in connection with FIGS. 47-49. Block 1330 describes a transparent polymeric bag-like conformal container with recirculation water with respect to a temperature controlled reservoir and pump as described in connection with FIG. 50. Line 1332 extends to another configuration described at block 1334 wherein a transparent polymeric conformal container is combined with a recirculating temperature controlled liquid such as water and a mechanical agitator as described in connection with FIG. 51. Line 1336 extends from block 1330 to block 1338 to describe another heat sink configuration which is the heat controlled aluminum heat sink described in connection with FIGS. 19-21. Line 1340 extends from block 1334 to another heat sink configuration described at block 1342. At that block, a transparent polymeric conformal container with a magnetic stirring assembly configuration is set forth as has been described in connection with FIGS. 52 and 53. Line 1344 extending from block 1338 leads to block 1346 representing another transparent polymeric bag-like conformal container configuration which incorporates a motor driven propeller agitator. This approach has been described in connection with FIGS. 54 and 55. Line 1348 extending from block 1342 and line 1350 extending from block 1346 lead to line 1352 and block 1354 indicating that a heat sink configuration has been selected. From block 1354 line 1356 extends to block 1358 indicating that where a transparent container is selected, a clinician may optionally provide a pattern of visible indicia adjacent its contact surface which corresponds with the starting pattern of visible indicia. That arrangement has been described in connection with FIGS. 56-58. As represented by line 1360 and block 1361 the outside of the contact surface of a transparent heat sink may be treated with a thin, transparent layer of thermochromic material which has a visually perceptible color cue at epidermis surface temperatures above a maximum value, for example, 40° C. With the emergence of this color at a region as described in connection with FIG. 59, the system can be shut down. Line 1362 extends from block 1361 to the optional arrangement set forth at block 1363. That option provides for the location of one or more temperature sensors on the heat sink container surface for the purpose of measuring liquid (water) temperature while it is being stirred. Such sensors should be displaced from the heat sink contact surface. Next, as represented at line 1364 and block 1366, an appropriate heat sink (water) temperature is determined taking into account the temperature drop at the interface between the epidermis surface and the heat sink contact surface. The temperature of the water within the polymeric bag-like container will be within a range of about 15° C. to 25° C. The procedure then continues as represented at line 1368 extending to block 1370. Block 1370 provides that the subcutaneous fat layer may be pre-cooled from the skin surface for a pre-cooling interval. This pre-cooling technique has been described in connection with FIG. 22. As represented at line 1372 and block 1374 the clinician may optionally form the heating channels for receiving implants utilizing a surgically blunt dissecting introducer device. Such a device has been described in conjunction with FIGS. 45 and 46. Line 1376 extends from block 1374 to introduce procedures for administering local anesthetic. It is preferred that the local anesthetic be administered by injection as opposed to diffusion through the epidermis and dermis. Where the agent is administered within the skin region determined for carrying out collagen shrinkage it is important that the electrical conductivity at the next subcutaneous tissue or fat layer not be enhanced. In a natural state, the electrical conductivity of this fat layer is substantially less than dermis thus, RF current flux paths will tend to remain in the dermis. As noted earlier herein, the more popular of anesthetic agents is lidocane combined with a normal saline diluent. That normal saline diluent will exhibit an electrical resistivity which is 50-60 ohm-centimeters which represents a relatively high conductivity with respect to that exhibited by the fat layer. Accordingly, it is preferred to utilize a diluent which does not enhance electrical conductivity. In general, a local anesthetic solution incorporating 0.8% lidocane with a diluent of 5% dextrose and water in combination with epinephrine in a ratio of 1:2000 may be employed. Thus, as represented at block 1378 where an infiltration local anesthetic is injected, the anesthetic agent is combined with a low electrical conductivity biocompatible diluent. On the other hand, as represented at line 1380 and block 1382 where the clinician elects to administer the local anesthetic as a nerve block remote from the skin region under consideration, then a conventional anesthetic agent combined with an isotonic saline diluent may be employed inasmuch as the anesthetic will be remote from RF current paths. The electrical characteristics of local anesthetics are considered in detail in U.S. Pat. No. 7,004,174 by Eggers, et al., issued Feb. 28, 2006 and incorporated herein by reference. A delay is called for subsequent to the administration of a local anesthetic. In this regard, line 1384 extends from block 1382 to block 1386 providing for a delay, t₂, for anesthetic effectiveness. It may be recalled that a diffusion delay, t₁, is required following, for instance, the topical application of adjuvants over the skin region of interest. That delay generally will be of shorter duration than the delay for anesthetic effectiveness. Accordingly, the clinician may wish to carry out the procedure of block 1274 subsequent to the procedure of blocks 1378 or 1382. At this stage in the procedure the practitioner will attach the electrode leads and resistor leads to the system controller as discussed in connection with FIGS. 41-44 and 80-83. Such connection is represented at line 1387 and block 1388. Following connection with the system controller, as represented at line 1389 and block 1390, an entrance incision is formed at each heating channel entrance location. The clinician then has the option of forming the heating channel utilizing the introducer device discussed at block 1374 or employing a bladed implant as discussed in connection with block 1302. Accordingly, as represented at line 1392, the clinician optionally may utilize a dissecting instrument to form heating channels commencing at each heating channel entrance location. Next, as represented at line 1396 and block 1398, an implant is inserted within each heating channel though the now open entrance location. The electrodes will be oriented for contact with the lower region of the dermis layer.

Line 1400 extends from block 1398 to describe the next option represented at block 1402. For this option, the heating channel is formed by a bladed implant while the implant is being positioned. Such a bladed implant has been described in connection with block 1302. For either implant option, as represented at line 1404 and block 1406, the clinician may control the length of implant insertion by observing the positioning indicia with respect to the channel entrance location incision. Such indicia has been described in connection with FIGS. 79A-79C.

As part of this positioning, the clinician also may verify implant location by palpation as represented at line 1408 and block 1410. Following such positioning, as represented at line 1412 and block 1414, a heat transferring liquid such as water or glycerol is applied to the skin region of interest. This fluid also serves as a lubricant permitting the movement of skin below an applied heat sink. In the latter regard, as represented at line 1416 and block 1418, the selected heat sink is positioned against the skin region epidermis and whatever agitator or recirculation system which is associated with it is actuated. As an aspect of heat sink positioning any pattern of visible indicia carried by it may be aligned with a skin carried starting pattern. Such an arrangement has been addressed in connection with FIGS. 56-58. With the positioning of the implants, as represented at line 1420 and block 1426 the controller associated with the cables will verify whether or not proper electrical connections have been made. In the event they have not, then as represented at line 1428 and block 1430 the operator will be cued as to the discrepancy and prompted to recheck connections. The program then returns to line 1420 as represented at line 1432. In the event of an affirmative determination to the query at block 1426, then the procedure continues as represented at line 1434 and block 1436 where the operator initiates auto-calibration of all temperature sensing resistor segments and any heater resistors with respect to setpoint temperature. Auto-calibration has been discussed above in connection with equations (2) and (3). When the setpoint temperature related resistance(s) have been developed, as represented at line 1438 and block 1440, the resistance value(s) associated with setpoint temperature(s) are placed in memory and the program continues as represented at line 1442 and block 1444. The query at block 1444 determines whether auto-calibration has been successfully completed. In the event that it has not, then as represented at line 1446 and block 1448 the controller provides an illuminated auto-calibration fault cue and, as represented at line 1450 and block 1452, it provides a prompt to recheck connection of cables and to replace any faulty implant. The program then loops to line 1434 as represented at line 1454. Returning to block 1444, where auto-calibration has been successfully completed, then as represented at line 1456 and block 1458 slight pressure or tamponade is applied over the skin region of interest through the selected heat sink. For example, such pressure has been described as being applied through a transparent glass plate in connection with inter alia, FIG. 55. In general, this pressure will be greater than 0 psi and does not need to be greater than 0.22 psi.

The program then commences to start the therapy as represented at line 1460 and block 1462 and described in connection with FIG. 77 with respect to either curve components 1096 or 1232. Variable power is applied to the electrodes in ramp control fashion over a controlled time interval, for example four minutes. During this ramp interval, as represented at line 1464 and block 1466 the clinician may visually monitor the extent of ongoing shrinkage. In this regard, note the eye station 704 in FIG. 55. The controller will determine whether an electrode of a given implant has reached setpoint temperature as represented at line 1468 and block 1470. Where such a setpoint temperature has been reached, then as represented at line 1472 and block 1474 power to the implant is reduced and applied in constant power fashion for a thermal soak interval, for example, one minute as described in connection with FIG. 77 at curve portions 1100 and 1234. When the determination at block 1470 is that an electrode of a given implant has not reached setpoint temperature, then as represented at line 1476 and block 1478, a determination is made as to whether the extent of shrinkage desired has been reached. In this regard, the desired extent of collagen shrinkage may be accomplished before the end of a predetermined therapy interval. Where that goal has not been reached, then as represented at line 1480 and block 1482 a query is posed as to whether the predetermined therapy interval has been completed. In the event that it has not, then as represented at line 1484 and block 1486 query is made as to whether the operator has initiated a stop therapy condition. This stopping of therapy may, for instance, be a consequence of a malfunction such as an unwanted burn condition or in the event a shrinkage goal has been reached before the termination of a therapy interval. In the event of a negative determination, then as represented at line 1488 the program loops to line 1476 and the queries which follow.

Returning to the query at block 1478, where the shrinkage goal has been reached, then as represented at line 1490 and block 1492, all electrodes are de-energized.

Returning to the query at block 1482, where the therapy interval is completed then as represented at lines 1494, 1490 and block 1492 all electrodes are de-energized. That condition also obtains where an affirmative response occurs in connection with the query at block 1486. In this regard, line 1496 extends to line 1490 and block 1492.

With the de-energization of all electrodes, as represented at line 1498 and block 1500 post therapy continued temperature control is carried out for a post therapy interval. That post therapy interval has been described in connection with FIG. 22. The post therapy interval may last, for example, about two minutes. Accordingly, as represented at line 1502 and block 1504 a determination is made as to whether the post therapy interval is completed. In the event that it is not, then as represented at line 1506 extending to line 1502, the program loops. If the post therapy interval is completed then as represented at line 1508 and block 1510 the selected heat sink configuration is removed with concomitant release of pressure, and the program continues as represented at line 1512 and block 1514. At this stage in the procedure, the clinician evaluates the extent of collagen shrinkage accomplished. As represented at line 1516 and block 1518 a query is posed as to whether an acceptable extent of shrinkage has been accomplished. In the event that it has not, then as represented at line 1520 and block 1522 the clinician restores and activates the heat sink configuration and, as represented at line 1524 and node A therapy is restarted. Node A reappears in FIG. 84G with line 1525 extending to line 1460.

Returning to the query posed at block 1518, where an acceptable extent of shrinkage has occurred, then as represented at line 1526 and block 1528 the implants are removed and, as indicated at line 1530 and block 1532 all entrance incisions are repaired. As represented at line 1534 and block 1536 therapy is then completed. However, as shown at line 1538 and block 1540, the clinician will carry out a post therapy review to determine the presence of successful neocollagenisis.

Implants of the invention also may be employed in treating capillary malformation which often is referred to as port wine stain (PWS). As discussed above in connection with publication 15, such lesions have been classified, for instance, utilizing video microscopy, three patterns of vascular ectasia being established; type 1 ectasia of the vertical loops of the capillary plexus; type 2 ectasia of the deeper, horizontal vessels in the capillary plexus; and type 3, mixed pattern with varying degrees of vertical and horizontal vascular ectasia. As additionally noted above, in general, due to the limited depth of laser therapy, only type 1 lesions are apt to respond to such therapy.

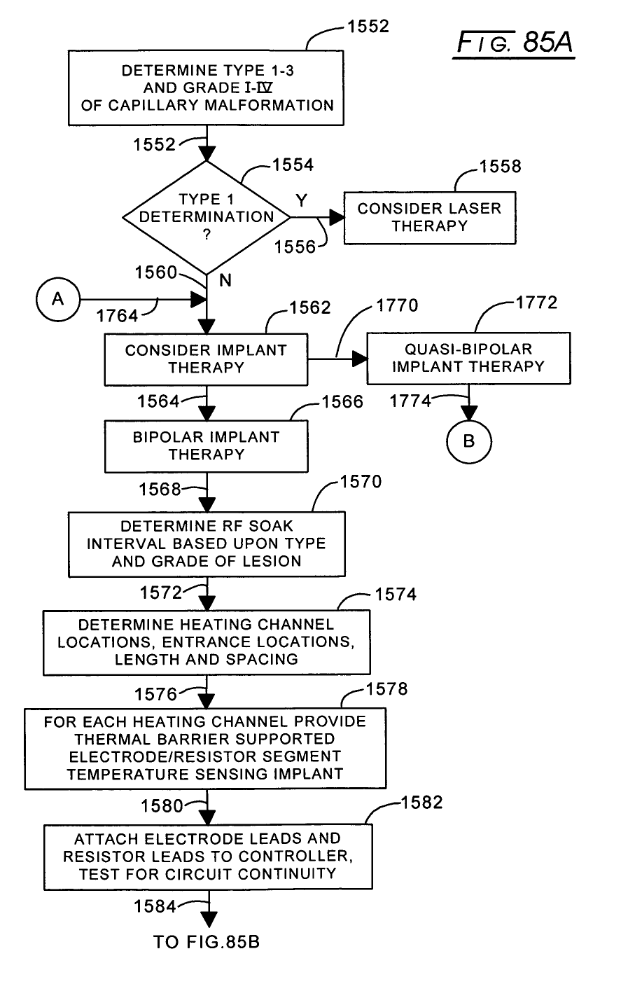

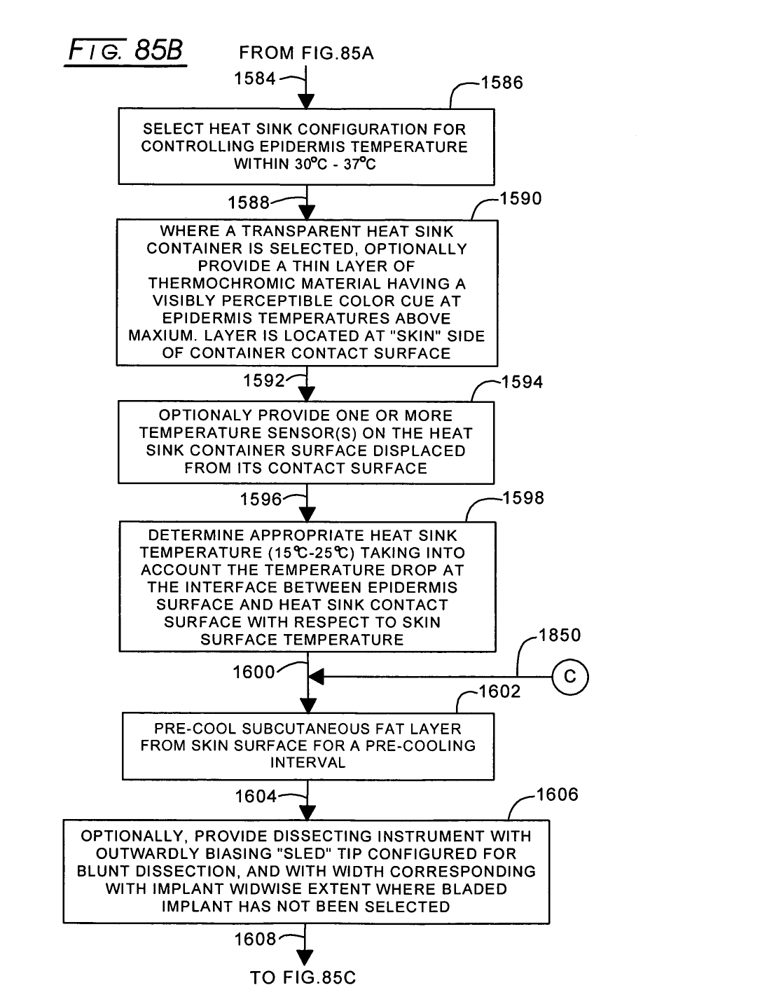

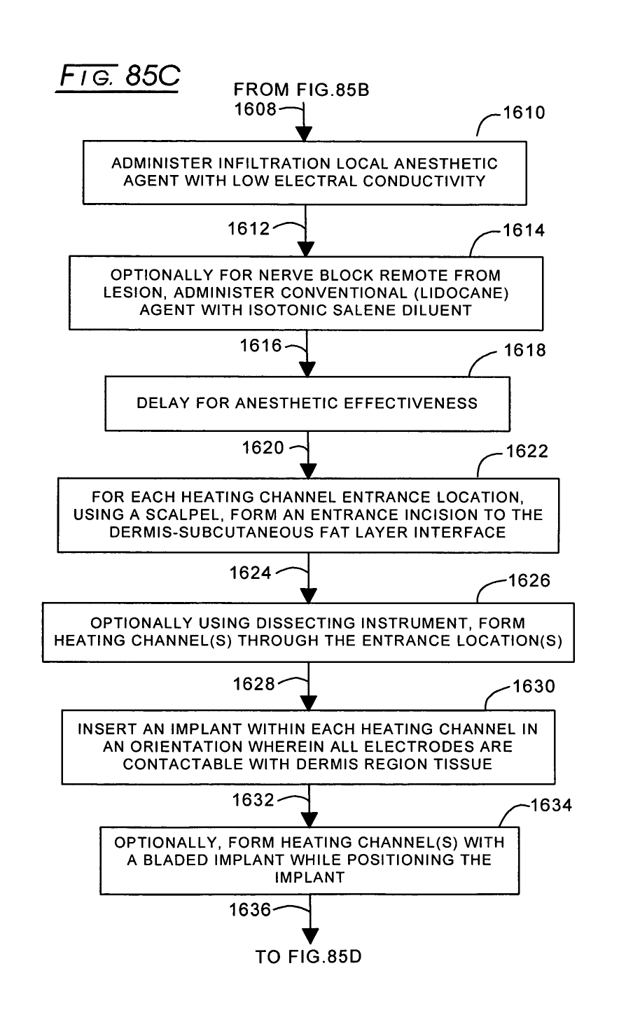

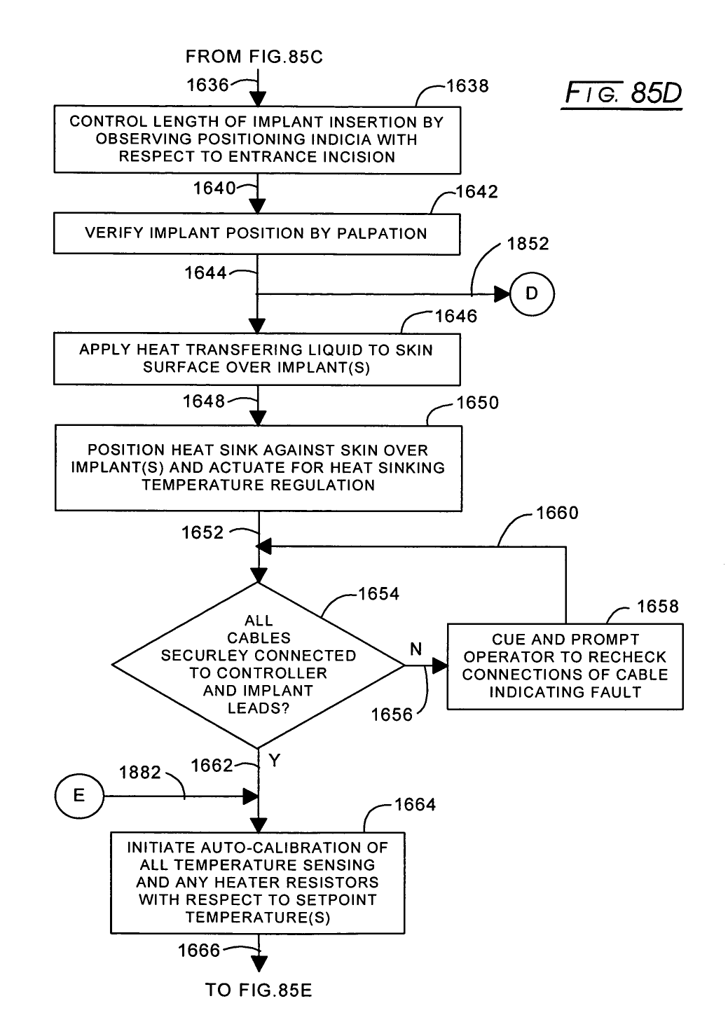

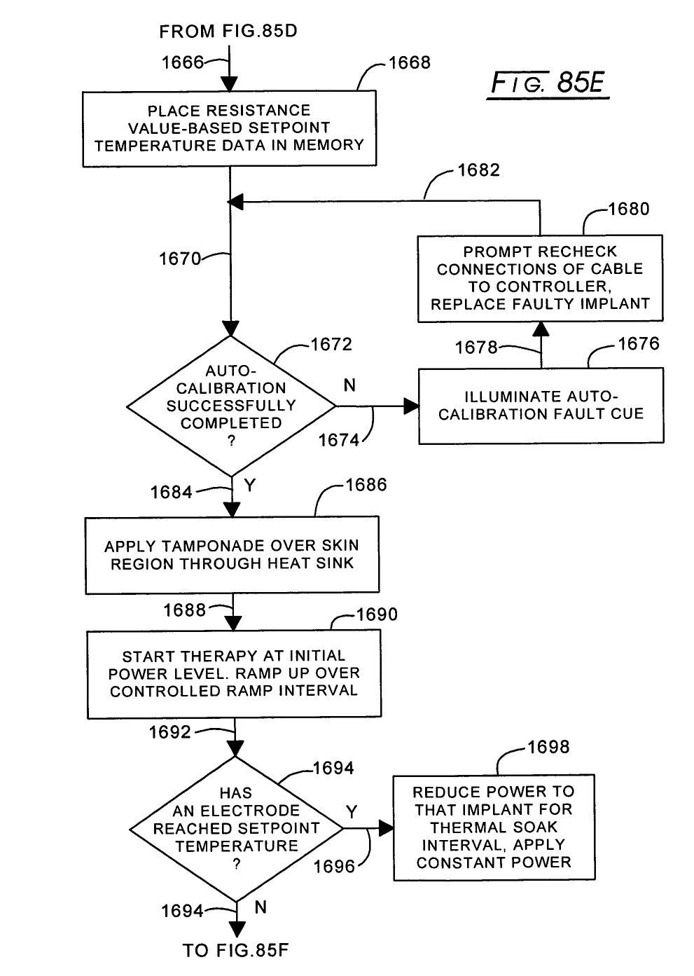

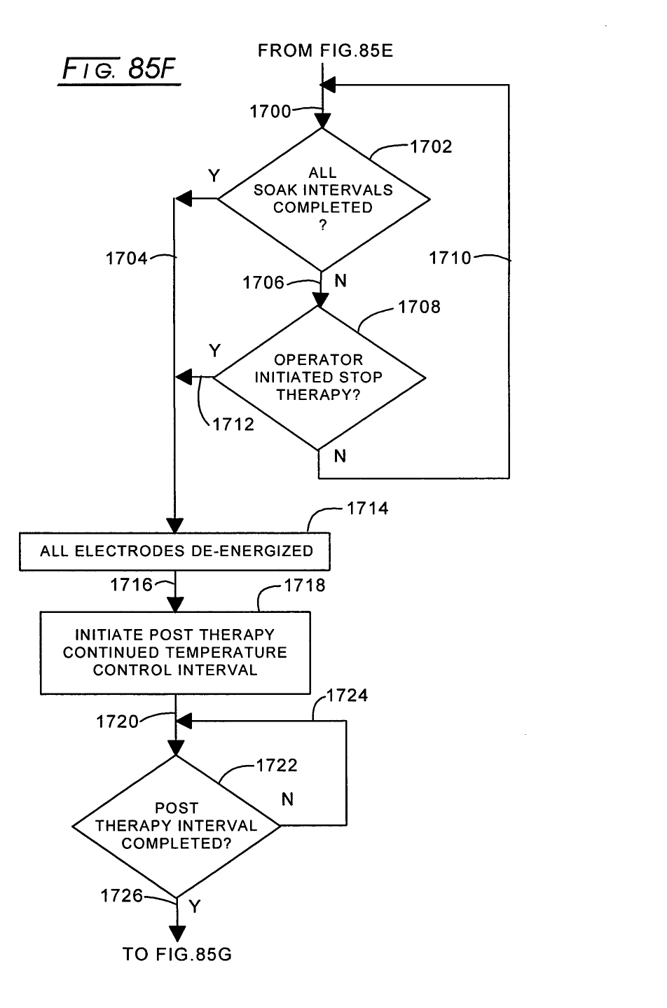

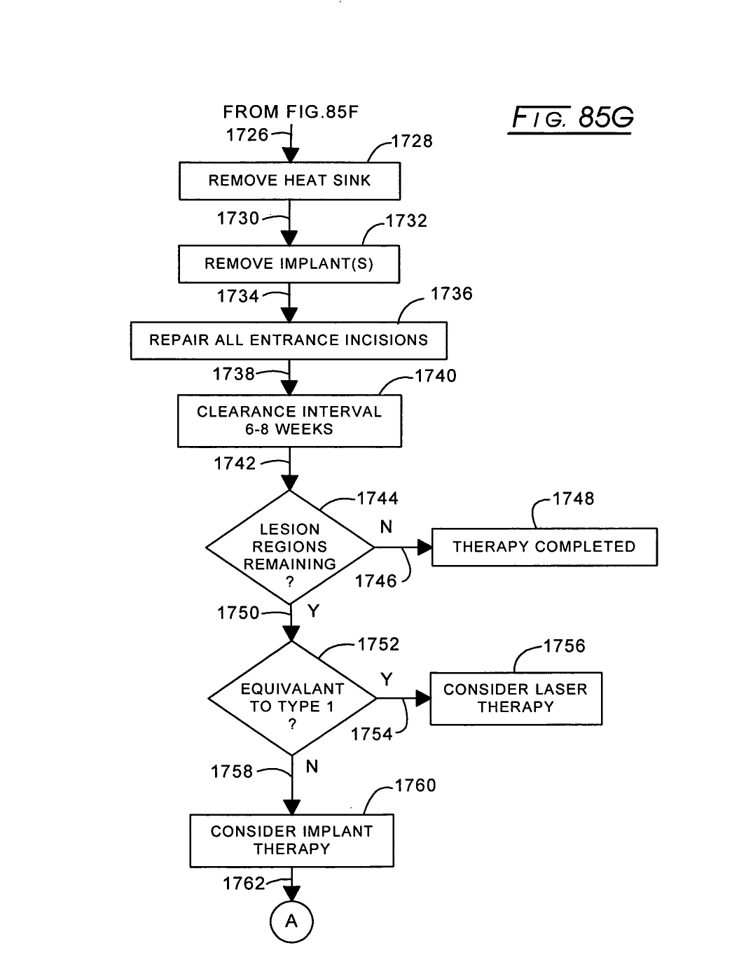

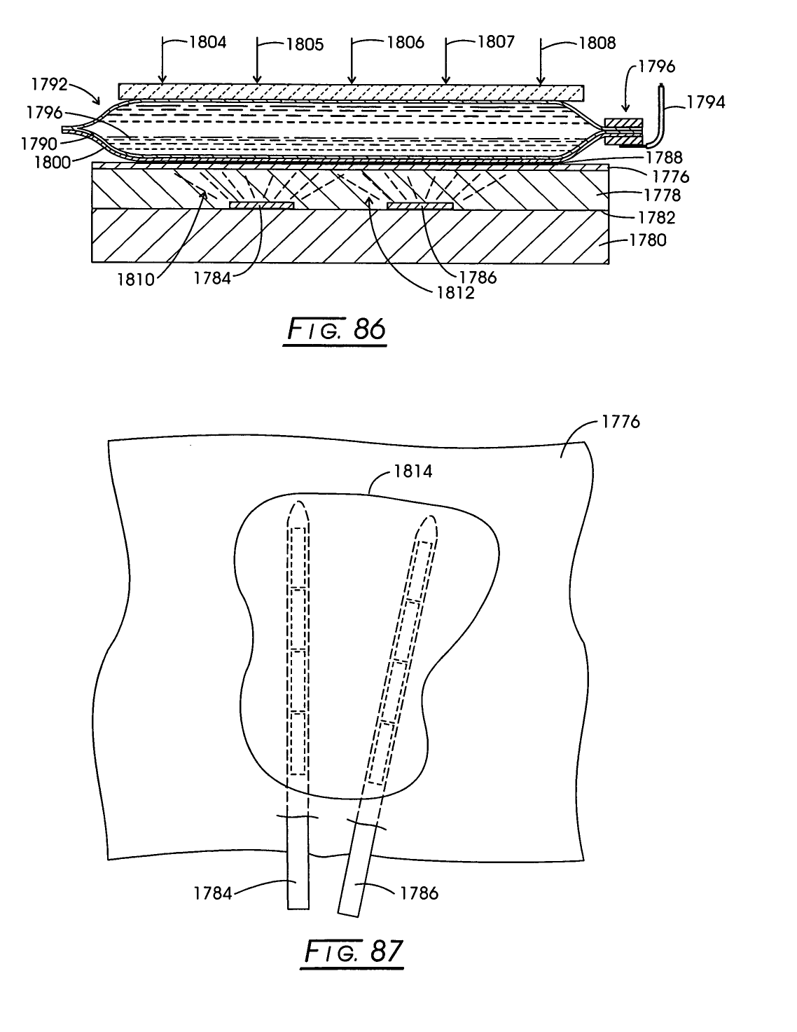

The capillary malformations (PWS) also are classified in accordance with their degree of vascular ectasia, four grades thereof being recognized as Grades I-IV. The grade categorizations are discussed above. FIGS. 85A-85G combine as labeled thereon to provide a process flowchart representing an initial appearance to the treatment of capillary malformation. Looking to FIG. 85A and block 1550, a determination is made of the type and Grade of the capillary malformation lesion. Then, as represented at line 1552 and block 1554, a query is posed as to whether a type 1 determination is at hand. If that is the case, then as represented at line 1556 and block 1558, the practitioner may want to consider the utilization of laser therapy. On the other hand, where the determination at block 1554 indicates that a type 1 lesion is not at hand, then as represented at line 1560 and block 1562 the practitioner will consider resort to implant therapy with implants as disclosed herein. Of the therapies available, utilizing these implants, as represented at line 1564 and block 1566, bipolar implant therapy utilizing radiofrequency energy may be elected. Energization of the electrodes in general will be provided as described in connection with curve 1090 as set forth in FIG. 77 but at a much lower setpoint temperature which will not adversely effect dermis tissue, i.e., that setpoint temperature will be atraumatic with respect to dermis. In general, such setpoint temperature will be in a range from about 45° C. to about 60° C. Once setpoint temperature is reached, then a thermal soak interval ensues as described at curve portion 1100. Accordingly, as represented at line 1568 and block 1570 the practitioner will determine a radiofrequency soak interval at lower radiofrequency power based upon the determined type and grade of lesion. Heating of the blood vessels of the lesion takes place to an extent evoking necrotic cauterization and subsequent dissipation (resorption) from the dermis. As this occurs, while the heating remains atraumatic to dermis, angiogenisis or the formation of new blood vessels will occur, typically without the regeneration of capillary malformation. Next, as indicated at line 1572 and block 1574 a determination is made as to the heating channel locations including the entrance locations and the length and spacing of the channels. Once the heating channels are determined, then as represented at line 1576 and block 1578 for each such heating channel there is provided a thermal barrier supported electrode/resistor segment temperature sensing implant. As discussed in connection with FIGS. 79A-79C, it is preferred that the implants will have the same overall length and retain a fixed number of electrodes and resistor segments, the electrodes varying in a common length. As represented at line 1580 and block 1582, the practitioner will attach electrode and resistor leads to controller cables. Circuit continuity may be tested at this juncture. The procedure continues with selection of a heat sink configuration as represented at line 1584 and block 1586. Generally the heat sink will maintain the epidermis surface temperature within a range of about 30° C. to about 37° C. Various heat sink configurations have been discussed above in connection with FIGS. 47-55. Should the heat sink selected be transparent, then as represented at line 1588 and block 1590 as an option, a layer of thermochromic material having a visibly perceptible color cue at epidermis surface temperatures above an elected maximum can be provided. The material layer will be located at the “skin” side of the container contact surface. Such material has been discussed above in connection with FIG. 59. Another option is represented at line 1592 and block 1594 wherein one or more temperature sensors may be located on the heat sink container surface displaced from its contact surface. In this same regard, as represented at line 1596 and block 1598 appropriate heat sink temperature is determined taking into account the temperature drop at the interface between the epidermis surface and the heat sink contact surface with respect to skin surface temperature. In general, the heat sink temperature will be in a range from about 15° C. to about 25° C. Line 1600 extends from block 1598 to block 1602 which provides that the practitioner may wish to pre-cool the subcutaneous fat layer from the skin surface for a pre-cooling interval. Where a bladed implant has not been provided as described in conjunction with block 1578, then the heating channel may be formed utilizing a blunt dissecting introducer instrument as discussed in connection with FIGS. 45 and 46. Where an infiltration form of local anesthetic is to be employed, then as represented at line 1608 and block 1610 the local anesthetic agent is one which exhibits a low electrical conductivity for reasons discussed with respect to block 1378 above. On the other hand, where a nerve block form of anesthetic agent is utilized, as represented at line 1612 and block 1614, a conventional anesthetic agent may be administered, for example, lidocaine in combination with an isotonic saline diluent. Time is required for the local anesthetic to become effective, thus, as represented at line 1616 and block 1618 a delay ensues awaiting anesthetic effectiveness. As the local anesthetic becomes effective then, as represented at line 1620 and block 1622, using a scalpel, before each heating channel entrance location, an entrance incision is made to the dermis-subcutaneous fat layer interface. Optionally, as represented at line 1624 and block 1626, a blunt dissecting instrument as provided at block 1606 may be employed for forming the heating channel(s) through the entrance incision(s). Once so formed, as represented at line 1628 and block 1630 an implant is inserted within each channel in an orientation wherein all electrodes are contactable with dermis. Generally, it has been found that where the implants are pre-connected to the controller cables insertion is more facilly carried out. Heating channels also may be formed in conjunction with the insertion of the implants where a bladed implant is employed as represented at line 1632 and block 1634. Such bladed implants have been described above in connection with FIGS. 67-70. Line 1636 extending to block 1638 from block 1634 indicates that the extent of implant insertion may be controlled by observing positioning indicia with respect to the entrance incision. Such indicia has been described above in connection with FIGS. 79A-79C. Next, as represented at line 1640 and block 1642, the position of the implants may be verified by palpation. In preparation for positioning of the heat sink, as represented at line 1644 and block 1646, a heat transferring liquid is applied to the skin surface over the implants, whereupon as represented at line 1648 and block 1650 a heat sink is positioned over the implants and is actuated for heat sinking temperature regulation. Heat sink configurations have been described above in connection with FIGS. 47-55 and FIG. 59. With the heat sink in position, as represented at line 1652 and block 1654, a determination is made as to whether all cables are securely connected to the controller as well as the implant leads. In the event that they are not, then as represented at line 1656 and block 1658 the practitioner is cued as well as prompted to recheck the connections of those cables indicating a fault. The program then loops to line 1652 as represented at line 1660. In the event of an affirmative determination with respect to block 1654, then as represented at line 1662 and block 1664, auto-calibration of all temperature sensing resistor segments with respect to setpoint temperature is carried out. Such auto-calibration has been discussed above in connection with blocks 1436, et seq. The auto-calibration procedure develops resistance values for each resistor segment which correspond with the reaching of setpoint temperature. As represented at line 1666 and block 1668, such resistance values representing setpoint temperature are placed in memory. The program continues as represented at line 1670 to the query posed at block 1672 determining whether the auto-calibration procedure has been successfully completed. In the event it has not been successfully completed, then as represented at line 1674 and block 1676 an auto-calibration fault cue is illuminated and, as represented at line 1678 and block 1680 the practitioner is prompted to recheck connections of cables to the controller and replace any faulty implant. The program then loops as represented at lines 1682 and 1670. In the event of an affirmative determination with respect to the query posed at block 1672, then as represented at line 1684 and block 1686 slight pressure is applied to the surface of the skin under treatment to assure appropriate electrode/dermis contact (tamponade). With such pressure application, as represented at line 1688 and block 1690, therapy is commenced by applying radiofrequency energy to the electrodes at an initial power level whereupon the energy is ramped-up over a control ramp interval. Such an approach is discussed above in connection with FIG. 77. However, for the instant therapy, the setpoint temperature is relatively low so as to remain atraumatic to the dermis, avoiding shrinking phenomena. The heat energy dosage is that providing for the necrotic coagulation of the blood vessel phenomena associated with capillary malformation. As represented at line 1692 and block 1694, a determination is made as to whether an electrode has reached setpoint temperature. In the event that it has reached that temperature, then as represented at line 1696 and block 1698 power is reduced to that implant and a thermal soak interval ensues preferably under constant power. In the event of the negative determination with respect to block 1694, then as represented at line 1700 and block 1702 a determination is made as to whether all soak intervals have been completed. In the event that they have, then the program continues as represented at line 1704. Where the soak intervals have not been completed, then as represented at line 1706 and block 1708 a determination is made as to whether the operator has initiated a stop therapy condition. In the event that the operator has not so initiated a stop, then the program loops as represented at line 1710 and 1700. Where the operator has initiated the stop therapy, then the procedure continues as represented at lines 1712 and 1704 which extends to block 1714. Block 1714 provides that all electrodes are de-energized, whereupon as represented at line 1716 and block 1718 the practitioner initiates post therapy continued temperature control for a selected interval. This is carried out by maintaining the function of the heat sink for this post therapy interval. The post therapy interval being initiated, as represented at line 1720 and block 1722 the program queries as to whether the post therapy interval is completed. In the event it has not been completed, then as represented at lines 1724 and 1720 the program loops. Where the post therapy interval has been completed, then as represented at line 1726 and block 1728 the heat sink is removed and as represented at line 1730 and block 1732 the implants are removed. Upon such removal as represented at line 1734 and block 1736 all entrance incisions are repaired. Next, as represented at line 1738 and block 1740 a clearance interval ensues, for instance, having a duration of about 6-8 weeks over which time the necroticly coagulated blood vessels causing the capillary malformation are naturally (resorption) absorbed. In general, the body function will tend to create normal vascularity in the treated region. As noted above, this is referred to as angiogenisis. Following the clearance interval, as represented at line 1742 and box 1744, a determination is made as to whether there are lesion regions remaining. In the event there are no such lesions remaining, then as represented at line 1746 and block 1748 therapy is completed. Where lesion regions do remain, then as represented at line 1750 and block 1752, a determination is made as to whether the remaining lesion region or regions are the equivalent to the earlier-discussed type 1 which are amenable to laser therapy. In the event that they are, then as represented at line 1754 and block 1756 the practitioner may consider laser therapy. Where the remaining lesions are not equivalent to a type 1 then, as represented at line 1758 and block 1760 the practitioner may consider implant therapy. With that consideration in mind, the procedure continues as represented at line 1762 and node A. Node A reappears in FIG. 85A in conjunction with line 1764 extending to line 1560. Line 1560 extends to block 1562 where the type of implant therapy is considered.

While bipolar implant therapy now has been described, as represented line 1770 and block 1772 the practitioner may also consider a quasi-bipolar implant therapy. Where that approach is elected, the program continues as represented at line 1774 and node B. With the quasi-bipolar approach, the electrode carrying electrode implants as described above are utilized at the dermis/next adjacent subcutaneous tissue interface. However, each implant performs individually with a dispersive return electrode. However, that return electrode is positioned on the skin immediately above the implant. Such a dispersion of radiofrequency current is quite short and advantageously away from the subcutaneous fat layer.

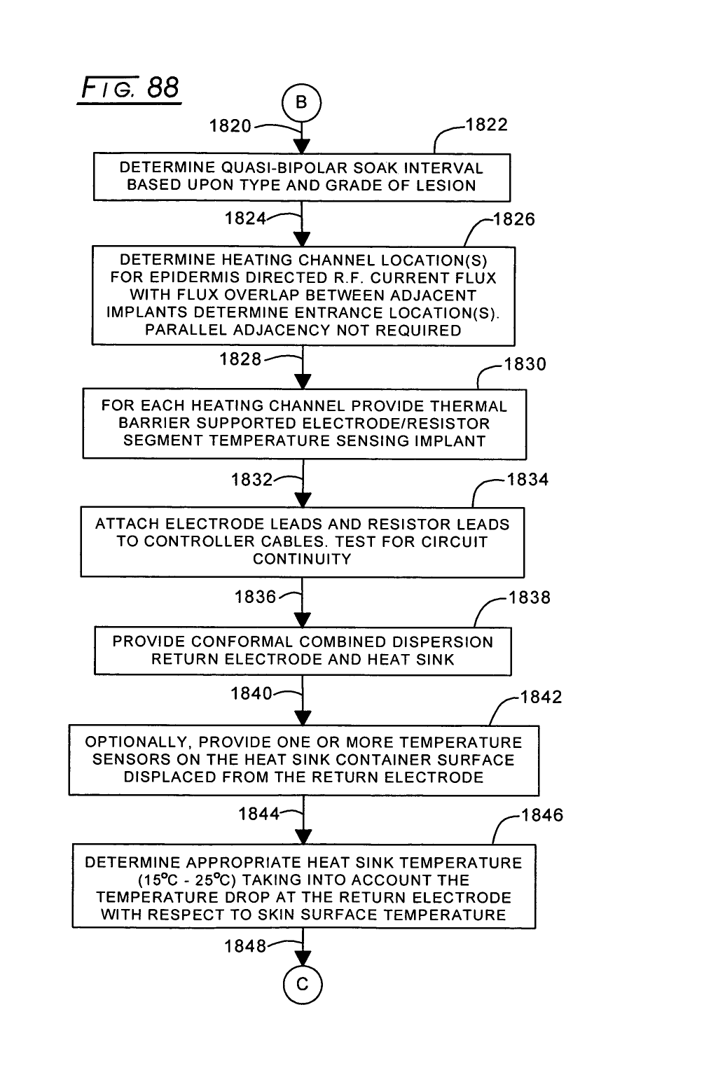

Referring momentarily to FIG. 86, schematically portrayed in epidermis 1776; dermis 1778 and next adjacent subcutaneous tissue or fat layer 1780. The interface between that layer and dermis is shown at 1782 and two implants, for example, as described in connection with FIGS. 79A-79C are shown at 1784 and 1786.

Positioned on top of the epidermis 1776 is a conformal diffusing return electrode 1788 which is fixed against contact surface 1790 of a conformal, liquid filled heat sink represented generally at 1792. Cable attachment to the return electrode 1788 is represented generally at 1794 which may form a portion of a bag or container clamping assembly represented generally at 1796. Liquid is symbolically shown at 1796. A thermal and electrical coupling liquid is located between the surface of epidermis 1776 and the conformal return electrode 1788. Where a capacitive coupling is developed between the electrodes of the implants 1784 and 1786 and the conformal electrode 1788, then the liquid 1800 need not be electrically conductive but only thermally conductive. The configuration of heat sink 1792 may vary somewhat and generally will be structured as one of the heat sinks described, for instance, in FIGS. 47-55. As before, pressure or tamponade may be applied from a rigid glass plate 1802 as represented by force arrows 1804-1808. With the quasi-bipolar arrangement shown, radiofrequency current flux will dispersively emanate from the electrodes of implants 1784 and 1786 to the return electrode 1788 as represented respectively by flux paths shown generally at 1810 and 1812. Note that they slightly overlap. An apparent advantage to this arrangement, particularly for capillary malformation (PWS) therapy is the tendency of energy concentration at the electrodes of the implants themselves at lower dermis locations.

Looking to FIG. 87, the schematic arrangement of FIG. 86 is revealed with portions removed in the interest of clarity. The boundary or border of the capillary malformation is shown at 1814 and note that the implants 1784 and 1786 may be located within heating channels which are not necessarily parallel inasmuch as current flow is upward through the dermis to the return electrode 1788.

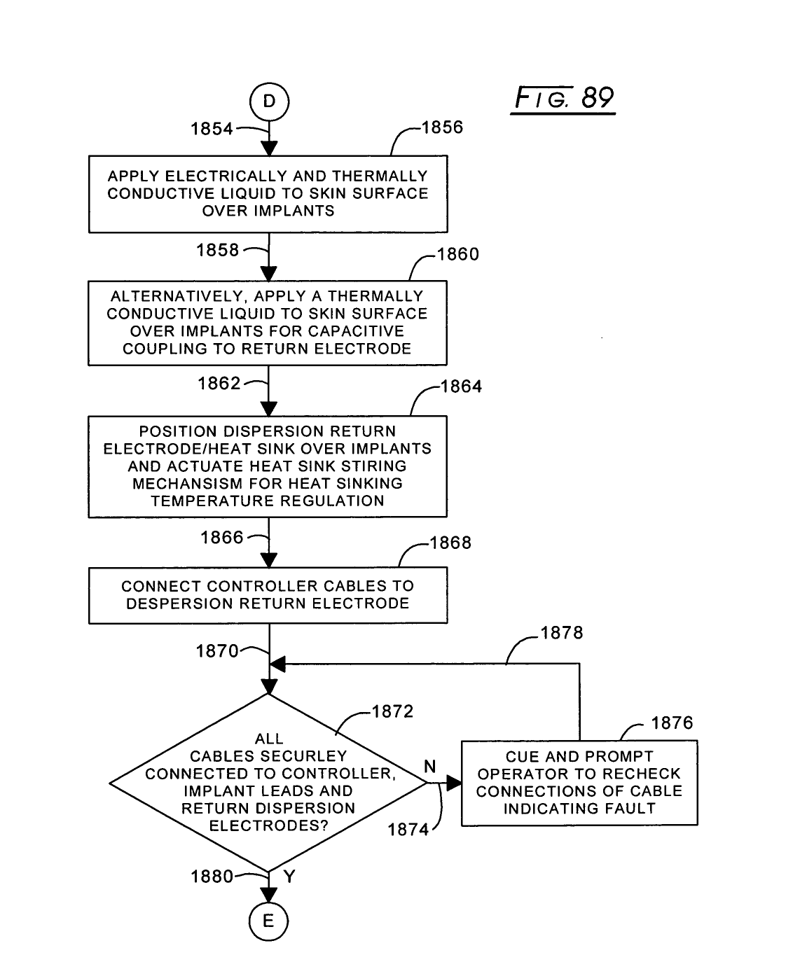

FIGS. 88 and 89 should be considered together in accordance with the labeling thereon. Looking to FIG. 88, node B reappears from FIG. 85A along with line 1820 and block 1822. It may be recalled from FIG. 85A that a determination has been made as to the type and Grade of the capillary malformation lesion and, in particular, whether it is a type 1. Block 1822 determines a quasi-bipolar soak interval based upon the type and Grade of lesion determined earlier. Next, as represented at line 1824 and block 1826 the heating channel locations as well as their entrance locations are determined for epidermis directed radiofrequency current flux with overlap as described in conjunction with FIG. 86. As discussed in connection with FIG. 87, parallel adjacency of the implants is not required. From block 1826, the procedure proceeds as represented at line 1828 and block 1830 which provides a thermal barrier supported electrode/resistor segment temperature sensing implant for each of the determined heating channels. These implants are attached to a control console via cables as represented at line 1832 and block 1834. As this occurs, there will be a test for circuit continuity. It may be recalled that it has been found to be more convenient during the procedure for inserting the implants at the interface such as described in FIG. 86 at 1582 to have the controller cables pre-connected. The procedure continues as represented at line 1836 and block 1838 providing a conformal/combined dispersion return electrode and heat sink. This combination has been described above in connection with FIG. 86, further option is represented at line 1840 and block 1842, where one or more temperature sensors may be provided on the heat sink container surface displaced from the return electrode for liquid temperature monitoring. In the latter regard, as represented at line 1844 and block 1846 a determination may be made of an appropriate heat sink temperature, for instance, within a range of from about 15° C. to about 25° C. taking into account the temperature drop at the return electrode with respect to the skin surface temperature. Next, as represented at line 1848 the program reverts to node C which reappears in FIG. 85B with a line 1850 extending to line 1600. This indicates that the procedure represented by blocks 1602-1642 are to be repeated. Line 1644 extending from block 1642 is seen to be intersected by line 1852 extending to a node D. Node D reappears in FIG. 89 in conjunction with line 1854 extending to block 1856. Block 1856 provides for the application of an electrically and thermally conductive liquid to the skin surface over the implants. This assures both thermal transfer to the heat sink and electrical transfer of radiofrequency current to the return electrode component of the heat sink. Alternately, as represented at line 1858 and block 1860 a thermally conductive liquid may be applied to this skin surface over the implants for providing capacitive coupling to the return electrode. Next, as represented at line 1862 and block 1864 the combined dispersion return electrode and heat sink as described in conjunction with FIG. 86 is positioned over the implants and the heat sink liquid stirring mechanism is actuated for heat sinking temperature regulation. Just prior to or subsequent to such positioning, as represented at line 1866 and block 1868 the controller cables are connected to the dispersion return electrode, whereupon, as represented at line 1870 and block 1872 a determination is made as to whether all cables are securely connected to the controller and implant leads as well as to the return dispersion electrode. In the event that they are not so properly connected, then as represented at line 1874 and block 1876 the operator is cued to that condition and prompted to recheck connections of those cables indicating a fault. The program then loops as represented at lines 1878 and 1870. Where the determination at block 1872 is that all cables are properly operative, then, as represented at line 1880 the program progresses to node E. Node E reappears at FIG. 85D in conjunction with line 1882 extending to line 1662 and the therapy continues to node A as described in connection with a bipolar approach to utilization of the implants.

Since certain changes may be made in the above apparatus and method without departing from the scope of the disclosure herein involved, it is intended that all matter contained in the above description or shown in the accompanying drawings shall be interpreted as illustrative and not in a limiting sense.

1. Implant apparatus for effecting a controlled heating of tissue at the region of the dermis from a location generally at the interface of dermis and next adjacent subcutaneous tissue, comprising:
a thermally insulative generally flat support having a support surface and an oppositely disposed insulative surface, said support having a lengthwise dimension extending between leading and trailing ends and a widthwise dimension along an active length;
an electrode circuit supported from said support surface having one or more electrodes energizable from a radiofrequency source to generate heat within tissue at the region of the dermis; and
a lead assemblage extending from each electrode to a lead contact region adjacent said support trailing end.

2. The implant apparatus of claim 1 in which:
said lead assemblage is electrically insulated at least where contactable with tissue.

3. The implant apparatus of claim 1 in which:
said electrode circuit is located upon an electrically insulative electrode support substrate having an outer surface and an oppositely disposed inner surface supported from said support surface and extending to said trailing end.

4. The implant apparatus of claim 3 further comprising:
one or more electrically energizable resistor segments with a resistor lead assemblage extending therefrom located upon the outer surface of an electrically insulative resistor support substrate having an inner surface supported at said flat support surface and extending over said trailing end to expose a portion of said resistor lead assemblage at said insulative surface generally opposite said lead contact region; and
said electrode substrate inner surface being supported over said resistor support substrate outer surface.

5. The implant apparatus of claim 4 in which:
said resistor lead assemblage is configured to provide a four-point electrical connection with each resistor segment.

6. The implant apparatus of claim 3 further comprising:
one or more electrically energizable resistor segments supported from said support surface each being located in general alignment and thermal exchange relationship with an oppositely disposed electrode; and
a resistor lead assemblage extending from each said resistor segment to said lead contact region.

7. The implant apparatus of claim 6 in which:
said one or more resistor segments are supported upon said substrate inner surface; and
said thermally insulative support is configured with an opening extending therethrough at said trailing end shaped to provide electrical contact access with said resistor lead assemblage.

8. The implant apparatus of claim 6 in which:
said resistor lead assemblage is configured to provide a four-point electrical connection with each resistor segment.

9. The implant apparatus of claim 6 in which:
said one or more resistor segments are configured to provide a thermal output; and
said resistor lead assemblage is configured to effect the generation of said thermal output.

10. The implant apparatus of claim 9 in which:
said thermally insulative support is configured with an opening extending therethrough at said trailing end shaped to provide electrical contact access with said resistor lead assemblage.

11. The implant apparatus of claim 3 in which:
said electrodes are formed of gold plated copper having a thickness of between about 0.0003 inch and about 0.0014 inch.

12. The implant apparatus of claim 1 in which:
said electrodes are formed of a metal having a thickness effective to promote the spreading dispersion of thermal energy into the region of dermis.

13. The implant apparatus of claim 1 in which:
said electrodes are formed with copper having a thickness of between about 0.005 inch and about 0.020 inch.

14. The implant apparatus of claim 6 in which:
said resistor segments are formed of copper having a thickness of between about 0.003 inch and about 0.0014 inch.

15. The implant apparatus of claim 6 in which:
said one or more resistor segments are formed of a metal exhibiting a temperature coefficient of resistance greater than about 2000 ppm/° C.

16. The implant apparatus of claim 3 in which:
said thermally insulative support comprises a polyimide material.

17. The implant apparatus of claim 1 in which:
said thermally insulative electrode support substrate comprises a polyetherimide resin.

18. The implant apparatus of claim 1 in which:
said thermally insulative support is formed of one or more polymeric materials having a thickness from about 0.02 inch to about 0.08 inch.

19. The implant apparatus of claim 1 in which:
said leading end of the thermally insulative support is surgically blunt.

20. The implant apparatus of claim 1 in which:
said leading end is slanted forwardly to an extent effective to provide a mechanical bias toward dermis when the implant is inserted into said interface.

21. The implant apparatus of claim 1 in which:
said thermally insulative generally flat support is configured with a bladed leading end effective to enter a skin entrance incision and guidably move under compressive urging along said interface between dermis and next adjacent subcutaneous tissue to form and be located within a heating channel.

22. The implant apparatus of claim 21 in which:
said bladed leading end is configured for blunt dissection along said interface.

23. The implant apparatus of claim 21 in which:
said leading end is slanted forwardly to an extent effective to provide a mechanical bias toward dermis when the implant is inserted into said interface.

24. The implant apparatus of claim 1 in which:
said lead assemblage is configured for effecting the radiofrequency energization of two or more electrodes of a common implant apparatus in bipolar fashion.

25. The implant apparatus of claim 1 in which:
said thermally insulative generally flat support lengthwise dimension is a fixed, consistent value; and
said electrode circuit has a fixed, consistent number of electrodes having a common length along said lengthwise dimension which may vary with respect to a given implant.

26. The implant apparatus of claim 25 in which:
said fixed, consistent value is about 7.5 inches.

27. The implant apparatus of claim 1 further comprising:
an adjuvant supported from said support surface releasable to disperse within dermis and effective when dispersed to lower the thermal transition temperature for carrying out the shrinkage of dermis or a component of dermis.

28. The implant apparatus of claim 1 further comprising:
implant insertion extent identifying visible indicia located forwardly from said flat support trailing end.

29. The method for effecting a controlled heating of tissue within the region of the dermis of skin, comprising the steps:
(a) determining a skin region for treatment;
(b) providing one or more heater implants each comprising a thermally insulative generally flat support having a support surface and an oppositely disposed insulative surface, a circuit mounted at the support surface having one or more electrodes;
(c) determining one or more heating channel locations along said skin region;
(d) locating each heater implant along a heating channel generally at the interface between dermis and next adjacent subcutaneous tissue in an orientation wherein said one or more electrodes are electrically contactable with dermis and in thermally insulative relationship with said next adjacent subcutaneous tissue;
(e) applying tamponade over at least a portion of said skin region to an extent effective to maintain substantially uniform and continuous electrical contact between dermis and said one or more electrodes;
(f) simultaneously controlling the temperature of the surface of skin within said region to an extent effective to protect the skin surface from thermal injury while permitting the derivation of effective therapeutic temperature at the said region of the dermis; and
(g) effecting an a radiofrequency energization of said electrodes toward a setpoint temperature.

30. The method of claim 29 in which:
step (b) provides two or more implants; and
step (g) effects said energization in bipolar fashion.

31. The method of claim 39 in which:
step (g) is carried out to effect a controlled shrinkage of dermis or a component of dermis.

32. The method of claim 29 in which:
step (g) is carried out to effect a therapeutic treatment of a capillary malformation.

33. The method of claim 29 further comprising the step:
(h) monitoring the temperature of said electrodes during step (g);

34. The method of claim 29 in which:
step (b) provides said circuit as having a polymeric substrate with an outward face supporting one or more electrodes, and an inward face supported from said support surface.

35. The method of claim 34 in which:
step (b) provides said flexible circuit as supporting one or more temperature sensors each having a temperature responsive condition adjacent to said inward face in thermal exchange adjacency with a said electrode; and
step (h) carries out said monitoring of temperature by monitoring the said temperature responsive condition of each temperature sensor.

36. The method of claim 35 in which:
step (b) provides each said flexible circuit supported temperature sensor as a resistor; and
step (h) carries out said monitoring of temperature in a manner wherein said temperature responsive condition is electrical resistance.

37. The method of claim 33 in which:
step (b) provides two or more implants;
step (g) effects said energization in bipolar fashion and reduces the power level to a bipolar electrode pair in response to a setpoint temperature attained input; and
step (h) derives said setpoint temperature attained input in correspondence with each bipolar electrode pair.

38. The method of claim 29 in which:
step (b) provides said circuit as a circuit having a polymeric substrate with an outward face supporting one or more said electrodes, and an inward face supporting one or more heater resistor segments generally aligned with said one or more electrodes, said inward face being adhesively coupled with said support surface; and
step (g) further effects a heat deriving energization of said heater resistor segments.

39. The method of claim 38 further comprising the step:
(h) monitoring the combined temperature of each electrode and resistor segment during step (g).

40. The method of claim 39 in which:
step (h) is carried out by intermittently monitoring the resistance value of each resistor segment.

41. The method of claim 40 in which:
step (h) further is carried out by comparing the monitored resistance value with a target value of resistance corresponding with a setpoint temperature.

42. The method of claim 41 in which:
step (b) provides three or more implants including two outwardly disposed border implants and one or more inwardly disposed implants, only said outwardly disposed border implants being configured with heater resistor segments; and
step (g) effects said radiofrequency energization of said electrodes in bipolar fashion.

43. The method of claim 42 in which:
step (g) effects said radiofrequency energization in a sequence of paired implants extending from a border implant to an opposite border implant under a duty cycle regimen.

44. The method of claim 43 in which:
step (g) effects said radiofrequency energization under about a 50% duty cycle.

45. The method of claim 43 in which:
step (g) effects a heat deriving energization of said heater resistor segments at said border implants to an extent effective to substantially equalize the thermal output of border implants with those of inwardly disposed implants.

46. The method of claim 29 in which:
step (f) is carried out with a container of liquid located against said skin region.

47. The method of claim 46 in which:
step (f) is carried out with a conformal polymeric container having a contact surface located against skin at said skin region.

48. The method of claim 47 in which:
step (e) is carried out by applying pressure at said skin region with said container.

49. The method of claim 47 in which:
step (f) is further carried out by locating heat transferring liquid intermediate the surface of skin at said skin region and the contact surface of the container.

50. The method of claim 47 in which:
step (f) is further carried out by effecting an agitation of liquid within said container adjacent skin at said skin region.

51. The method of claim 47 in which:
step (f) is carried out with liquid within said container at a temperature between about 15° C. and about 25° C.

52. The method of claim 31 in which:
step (f) is carried out with a conformal polymeric container having a transparency effective to permit viewing of skin surface at said skin region.

53. The method of claim 52 further comprising the steps:
(i) providing a pattern of visible indicia at said skin region prior to steps (e), (f) and (g), and providing a corresponding pattern of visible indicia adjacent said container contact surface, and
(j) monitoring the extent of skin shrinkage during step (g) by comparing said pattern of visible indicia at said skin region with said pattern of visible indicia at said container contact surface.

54. The method of claim 29 in which:
step (f) controls the temperature of the skin within said region within a temperature range of from about 30° C. to about 37° C.

55. The method of claim 29 in which:
step (f) is carried out with a temperature controlled metal assembly having an electrically insulative contact surface which is located in thermal exchange relationship with the surface of skin at said skin region.

56. The method of claim 29 further comprising the step:
(j) precooling said next adjacent subcutaneous tissue through the surface of skin at said skin region prior to steps (d) through (g).

57. The method of claim 29 in which:
step (f) is continued subsequent to step (h) for an interval effective to alter the temperature of heated dermis toward human body temperature.

58. The method of claim 29 in which:
step (b) provides three or more implants;
step (g) effects said energization in bipolar fashion under a duty cycle regimen.

59. The method of claim 29 further comprising the steps:
(k) providing a current diffusing return electrode; and
(l) positioning the return electrode in electrical return relationship against epidermis over those implants located by step (d); and
wherein step (g) effects said radiofrequency energization between the electrode or electrodes of said one or more heater implants and said return electrode to effect said controlled heating of tissue.

60. The method of claim 59 in which:
step (k) provides the return electrode as an electrically conductive conformal surface.

61. The method of claim 59 in which:
step (l) is further carried out by locating an energy transferring liquid between the return electrode and epidermis.

62. The method of claim 60 in which:
step (k) provides the return electrode as a conformal polymeric container of liquid functioning as a heat sink; and
step (e) is carried out of applying pressure at said skin region with said container.

63. The method of claim 60 in which:
step (g) is carried out to effect a therapeutic treatment of a capillary malformation.

64. The method of claim 63 in which:
step (g) is carried out to effect an irreversible vascular coagulation with a setpoint temperature atraumatic to dermis.

65. The method of claim 31 further comprising the step:
(m) administering an adjuvant generally to dermis at said skin region effective to lower the thermal transition temperature for carrying out the shrinkage of dermis or a component of dermis.

66. The method of claim 65 in which:
step (m) administers said adjuvant topically at said skin region.

67. The method of claim 65 in which:
step (b) provides one or more implants as carrying said adjuvant at a location for dispersion within dermis from the heating channel.

68. The method of claim 29 in which:
step (b) provides two or more heater implants wherein said thermally insulative generally flat support exhibits a lengthwise dimension which is a fixed, consistent value, and said circuit has a fixed consistent number of electrodes having a common length which may vary among given implants.

69. The method of claim 68 in which:
step (b) provides said two or more implants as exhibiting a lengthwise dimension of about 7.5 inches.

70. The method of claim 29 in which:
step (b) provides said one or more heater implants with one or more electrodes formed of a metal having a thickness effective to promote the spreading dispersion of thermal energy into the region of dermis.

71. The method of claim 70 in which:
step (b) provides said one or more implants with one or more electrodes formed with copper having a thickness of between about 0.005 inch and about 0.020 inch.

72. The method for effecting a controlled heating of tissue within the region of the dermis of skin, comprising the steps:
(a) determining a skin region for treatment;
(b) providing two or more heater implants each comprising a thermally insulative generally flat support having a support surface and an oppositely disposed insulative surface, the support having a lengthwise dimension extending between leading and trailing ends, a widthwise dimension, a circuit mounted at the support surface having one or more electrodes;
(c) determining two or more heating channel locations at said skin region, each having a channel entrance location;
(d) forming an entrance incision at each channel entrance location;
(e) inserting a heater implant leading end through each entrance incision to locate it within a heating channel, the trailing end remaining outside the surface of said skin region, and the one or more electrodes being located for contact with adjacent dermis;
(f) applying tamponade over at least a portion of said skin region to an extent effective to maintain uniform electrical contact between the one or more electrodes of each implant and adjacent dermis;
(g) applying bipolar radiofrequency energization to the one or more electrodes of the inserted implants from the trailing ends thereof for a therapy interval; and
(h) removing the implant active area through the corresponding entrance incision.

73. The method of claim 72 further comprising the step:
(i) simultaneously with step (g) controlling the temperature of the surface of skin within said skin region to an extent effective to protect the skin surface from thermal injury.

74. The method of claim 73 in which:
step (i) controls the temperature of the skin surface within said region within a temperature range of from about 37° C. to about 40° C.

75. The method of claim 72 in which:
step (g) is carried out to effect a controlled shrinkage of dermis or a component of dermis.

76. The method of claim 72 in which:
step (g) is carried out to effect a therapeutic treatment of a capillary malformation.

77. The method of claim 75 further comprising the step:
(j) during and/or after step (g) and before step (h) determining an extent of skin shrinkage.

78. The method of claim 77 in which:
step (j) provides a pattern of visible indicia at said skin region prior to step (f) and visually determines the extent of relative movement of said indicia.

79. The method of claim 73 in which:
step (i) is continued subsequent to step (g) for an interval effective to alter the temperature of heated dermis toward human body temperature.

80. The method of claim 72 further comprising the step:
(k) precooling the next adjacent subcutaneous tissue to dermis through the surface of skin at said skin region prior to steps (d) through (h).

81. The method of claim 73 in which:
step (i) is carried out with a liquid containing conformal polymeric container having a contact surface located against skin at said skin region.

82. The method of claim 81 in which:
step (i) promotes a thermal exchange by agitation of said liquid adjacent said contact surface.

83. The method of claim 81 in which:
step (i) is further carried out by locating a heat transferring liquid lubricant intermediate the surface of skin at said skin region and the contact surface of the container.

84. The method of claim 73 in which:
step (i) is carried out with a temperature controlled metal heat sink having an electrically insulated contact surface which is located in thermal exchange relationship with the surface of skin at said skin region.

85. The method of claim 75 in which:
step (g) is carried out after having generally predetermined said therapy interval with respect to a desired extent of skin shrinkage and setpoint temperature.

86. The method of claim 76 further comprising the step:
(p) administering an adjuvant generally to dermis at said skin region effective to lower the thermal transition temperature for carrying out the shrinkage of dermis or a component of dermis.

87. The method of claim 86 further comprising the step:
step (b) provides one or more implants as carrying said adjuvant at a location for dispersion within dermis from the heating channel.

88. The method of claim 86 in which:
the thermal transition temperature lowering adjuvant of step (l) is one or more of salt, an enzyme, a detergent, a lipophile, a denaturing solvent, an organic denaturant, and acidic solution, or a basic solution.

89. The method of claim 88 wherein the enzyme is one or more of hyaluronidase, lysozyme, muramidase, or collagenase.

90. The method of claim 86 wherein said adjuvant is administered one or more of topically, transdermally, intradermally, subdermally, or hypodermally.

91. The method of claim 88 wherein said adjuvant is administered subdermally by release from a heater implant.

92. The method of claim 72 in which:
step (b) provides said two or more heater implants wherein said thermally insulative generally flat support lengthwise dimension is a fixed, consistent value, and said circuit has a fixed, consistent number of electrodes having a common length which may vary among given implants.

93. The method of claim 92 in which:
step (b) provides said two or more implants as having a flat support exhibiting a lengthwise dimension of about 7.5 inches.

94. The method of claim 72 in which:
step (b) provides said two or more implants with one or more electrodes formed of a metal having a thickness effective to promote the spreading dispersion of thermal energy into the region of dermis.

95. The method of claim 94 in which:
step (b) provides said two or more implants with one or more electrodes formed with copper having a thickness of between about 0.005 inch and about 0.020 inch.

96. The method of claim 72 in which:
step (b) provides said two or more implants with visible insertion indicia located forwardly from said flat support trailing end with a configuration effective to determine the extent of insertion of the implant within a heating channel.

97. The method of claim 96 in which:
step (e) inserts a heater implant within a heating channel to an extent identified by visually comparing said insertion indicia with said entrance incision.

98. The method of claim 76 in which:
step (g) is carried out to effect an irreversible vascular coagulation with a setpoint temperature and therapy interval atraumatic to dermis.

99. The method of claim 98 in which:
step (g) is carried out with a setpoint temperature within the range from about 45° C. to about 60° C.

100. A method for thermally remodeling skin, the improvement of which comprises remodeling skin in the presence of an effective amount of a collagen thermal transition temperature lowering adjuvant.

101. The method of claim 100 wherein the thermal transition temperature lowering adjuvant is one or more of a salt, an enzyme, a detergent, a lipophile, a denaturing solvent, an organic denaturant, an acidic solution, or a basic solution.

102. The method of claim 101 wherein the enzyme is one or more of hyaluronidase, lysozyme, muramidase, or collagenase.

103. The method of claim 101 wherein the denaturing solvent is one or more of an alcohol, an ether, monomethyl sulfoxide or DMSO.

104. The method of claim 101 wherein the organic denaturant is urea.

105. The method of claim 101 wherein two or more thermal transition temperature lowering adjuvants are present in a therapeutically effective combination.

106. The method of claim 100 wherein said adjuvant is administered one or more of topically, transdermally, intradermally, subdermally, or hypodermally.

107. The method of claim 106 wherein said adjuvant is administered subdermally by release from a heater implant.

108. The method for effecting a controlled heating of a capillary malformation within a skin region comprising the steps:
(a) determining the degree of vascular ectasia at said region;
(b) providing one or more heater implants each comprising a thermally insulative generally flat support having a support surface and an oppositely disposed insulative surface, the support having an active length, a circuit mounted at the support surface having one or more electrodes along the active length;
(c) determining one or more heating channel locations within said region each having an entrance location;
(d) locating each heater implant along a heating channel generally at the interface between dermis and next adjacent subcutaneous tissue in an orientation wherein said one or more electrodes are electrically contactable with dermis and in thermally insulative relationship with said next adjacent subcutaneous tissue;
(e) applying tamponade over at least a portion of said skin region to an extent effective to maintain substantially uniform and continuous electrical contact between dermis and said one or more electrodes;
(f) simultaneously controlling the temperature of the surface of skin within said region to an extent effective to protect the skin surface from thermal injury while permitting the derivation of effective therapeutic temperature at the said skin region dermis; and
(g) effecting a radiofrequency energization of said electrodes heating them toward a setpoint temperature atraumatic to dermis while effecting an irreversible vascular coagulation at the skin region.

109. The method of claim 108 in which:
step (g) effects said energization of said electrodes toward a setpoint temperature within a range of between about 45° C. and about 60° C.

110. The method of claim 108 furthering comprising the step:
(h) monitoring the temperature of each said electrode during step (g).

111. The method of claim 110 in which:
step (b) provides said implants as having one or more temperature sensors, each having a temperature responsive condition corresponding with the temperature of an electrode; and
step (h) carries out the monitoring of temperature by monitoring said temperature responsive condition.

112. The method of claim 108 in which:
step (f) is carried out with a container of liquid located against said skin region.

113. The method of claim 112 in which:
step (f) is carried out with a conformal polymeric container having a contact surface located against skin at said skin region.

114. The method of claim 113 in which:
step (e) is carried out by applying pressure at said skin region with said container.

115. The method of claim 108 in which:
step (b) provides two or more implants; and
step (g) effects said energization in bipolar fashion.

116. The method of claim 108 further comprising the steps:
(i) providing a current diffusing return electrode; and
(j) positioning the return electrode in electrical return relationship against epidermis over those implants heated by step (d); and
wherein step (g) effects said radiofrequency energization between the electrode or electrodes of said one or more heater implants and said return electrode to effect controlled heating at the capillary malformation.

117. The method of claim 108 further comprising the steps:
(k) subsequent to step (g) removing said one or more implants from each heating channel;
(l) waiting a clearance interval at least effective for the resorption of tissue at said skin region which has undergone irreversible vascular coagulation; and
(m) then repeating step (a).

118. The method of claim 117 further comprising the steps:
(n) where step (m) determines that any remaining capillary malformation is equivalent to a type 1 lesion, treating the remaining capillary malformation using laser-based therapy.
